(19) United States
(12) Patent Application Publication
(10) Pub. No.: US 2024/0269113 A1
(43) Pub. Date: Aug. 15, 2024

(54) 3-(2-(DIMETHYLAMINO)ETHYL)-1H-INDOL-4-yl DERIVATIVES

(71) Applicant: Compass Pathfinder Limited, Altrincham (GB)

(72) Inventor: John Richard MORPHY, Altrincham (GB)

(21) Appl. No.: 18/420,562

(22) Filed: Jan. 23, 2024

Related U.S. Application Data
(60) Provisional application No. 63/457,469, filed on Apr. 6, 2023.
(60) Provisional application No. 63/481,385, filed on Jan. 24, 2023.

(51) Int. Cl. A61K 31/4045; A61K 31/427; A61K 31/428; A61K 31/4439; A61K 31/695; C07D 209/14; C07D 401/12; C07D 403/12; C07D 417/12; C07F 7/08

(52) U.S. Cl. CPC A61K 31/4045; A61K 31/427; A61K 31/428; A61K 31/4439; A61K 31/695; C07D 209/14; C07D 401/12; C07D 403/12; C07D 417/12; C07F 7/0812

(57) ABSTRACT

Provided herein are compounds of Formula (I)

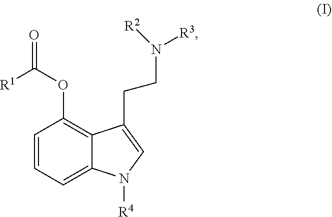

(I), a compound selected from any of the compounds in Table 1, Table 2, Table 3, Table 4 or Table 5, or a pharmaceutically acceptable salt or deuterated form thereof, wherein R¹, R², R¹ and R⁴ are defined herein. Also provided herein are pharmaceutical compositions comprising a compound of formula (I), or a compound selected from any of the compounds in Table 1, Table 2, Table 3, Table 4 or Table 5, or pharmaceutically acceptable salt or deuterated form thereof, and methods of using a compound of formula (I) or pharmaceutically acceptable salt or deuterated form thereof, e.g., in treating 5-HT₂A receptor associated diseases or disorders.

### CROSS-REFERENCE TO RELATED APPLICATIONS

This application claims priority to U.S. Provisional Application No. 63/481,385, filed, Jan. 24, 2023, and U.S. Provisional Application No. 63/457,469, filed Apr. 6, 2023, the disclosure of each of which is incorporated by reference in its entirety for all purposes.

### BACKGROUND

Over 50% of adults in the United States will be diagnosed with a psychiatric disorder at some point in their lifetime. Nearly 1 in 5 suffer from mental illness, and nearly 1 in 25 are afflicted with severe mental illness, such as major depression, schizophrenia, or bipolar disorder.

Psychedelics show promising activity in treating mental illness. New psychedelic compounds are needed for treating mental illness.

### SUMMARY

In some embodiments, this disclosure provides a compound of Formula (I)

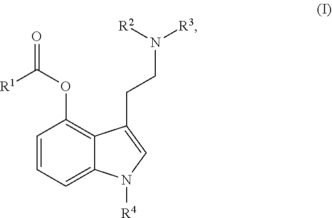

or a pharmaceutically acceptable salt or deuterated form thereof, wherein R¹, R², R³ and R⁴ are defined herein.

In some embodiments, this disclosure provides a compound selected from any of the compounds in Table 1, Table 2, Table 3, Table 4 or Table 5, or a pharmaceutical salt or deuterated form thereof.

### DETAILED DESCRIPTION

The terms below, as used herein, have the following meanings, unless indicated otherwise:

“Cyano” refers to the —CN radical.

“Hydroxy” or “hydroxyl” refers to the —OH radical.

“Oxo” refers to the ═O substituent.

“Alkyl” or “alkyl group” refers to a fully saturated, straight or branched hydrocarbon chain radical having from one to twelve carbon atoms, and which is attached to the rest of the molecule by a single bond. Alkyls comprising any number of carbon atoms from 1 to 12 are included. An alkyl comprising up to 12 carbon atoms is a C₁-C₁₂ alkyl, an alkyl comprising up to 10 carbon atoms is a C₁-C₁₀ alkyl, an alkyl comprising up to 6 carbon atoms is a C₁-C₆ alkyl and an alkyl comprising up to 5 carbon atoms is a C₁-C₅ alkyl. A C₁-C₅ alkyl includes C₅ alkyls, C₄ alkyls, C₃ alkyls, C₂ alkyls and C₁ alkyl (i.e., methyl). A C₁-C₆ alkyl includes all moieties described above for C₁-C₅ alkyls but also includes C₆ alkyls. A C₁-C₁₀ alkyl includes all moieties described above for C₁-C₅ alkyls and C₁-C₆ alkyls, but also includes C₇, C₈, C₉ and C₁₀ alkyls. Similarly, a C₁-C₁₂ alkyl includes all the foregoing moieties, but also includes C₁₁ and C₁₂ alkyls. Non-limiting examples of C₁-C₁₂ alkyl include methyl, ethyl, n-propyl, i-propyl, sec-propyl, n-butyl, i-butyl, sec-butyl, t-butyl, n-pentyl, t-amyl, n-hexyl, n-heptyl, n-octyl, n-nonyl, n-decyl, n-undecyl, and n-dodecyl. Unless stated otherwise specifically in the specification, an alkyl group can be optionally substituted.

“Alkylene” or “alkylene chain” refers to a fully saturated, straight or branched divalent hydrocarbon chain radical, and having from one to twelve carbon atoms. Non-limiting examples of C₁-C₁₂ alkylene include methylene, ethylene, propylene, n-butylene, and the like. The alkylene chain is attached to the rest of the molecule through a single bond and to the radical group through a single bond. The points of attachment of the alkylene chain to the rest of the molecule and to the radical group can be through one carbon or any two carbons within the chain. Unless stated otherwise specifically in the specification, an alkylene chain can be optionally substituted.

“Alkenyl” or “alkenyl group” refers to a straight or branched hydrocarbon chain radical having from two to twelve carbon atoms, and having one or more carbon-carbon double bonds. Each alkenyl group is attached to the rest of the molecule by a single bond. Alkenyl group comprising any number of carbon atoms from 2 to 12 are included. An alkenyl group comprising up to 12 carbon atoms is a C₂-C₁₂ alkenyl, an alkenyl comprising up to 10 carbon atoms is a C₂-C₁₀ alkenyl, an alkenyl group comprising up to 6 carbon atoms is a C₂-C₆ alkenyl and an alkenyl comprising up to 5 carbon atoms is a C₂-C₅ alkenyl. A C₂-C₅ alkenyl includes C₅ alkenyls, C₄ alkenyls, C₃ alkenyls, and C₂ alkenyls. A C₂-C₆ alkenyl includes all moieties described above for C₂-C₅ alkenyls but also includes C₆ alkenyls. A C₂-C₁₂ alkenyl includes all moieties described above for C₂-C₅ alkenyls and C₂-C₆ alkenyls, but also includes C₇, C₈, C₉ and C₁₂ alkenyls. Similarly, a C₂-C₁₂ alkenyl includes all the foregoing moieties, but also includes C₁₁ and C₁₂ alkenyls. Non-limiting examples of C₂-C₁₂ alkenyl include ethenyl (vinyl), 1-propenyl, 2-propenyl (allyl), iso-propenyl, 2-methyl-1-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1-heptenyl, 2-heptenyl, 3-heptenyl, 4-heptenyl, 5-heptenyl, 6-heptenyl, 1-octenyl, 2-octenyl, 3-octenyl, 4-octenyl, 5-octenyl, 6-octenyl, 7-octenyl, 1-nonenyl, 2-nonenyl, 3-nonenyl, 4-nonenyl, 5-nonenyl, 6-nonenyl, 7-nonenyl, 8-nonenyl, 1-decenyl, 2-decenyl, 3-decenyl, 4-decenyl, 5-decenyl, 6-decenyl, 7-decenyl, 8-decenyl, 9-decenyl, 1-undecenyl, 2-undecenyl, 3-undecenyl, 4-undecenyl, 5-undecenyl, 6-undecenyl, 7-undecenyl, 8-undecenyl, 9-undecenyl, 10-undecenyl, 1-dodecenyl, 2-dodecenyl, 3-dodecenyl, 4-dodecenyl, 5-dodecenyl, 6-dodecenyl, 7-dodecenyl, 8-dodecenyl, 9-dodecenyl, 10-dodecenyl, and 11-dodecenyl. Unless stated otherwise specifically in the specification, an alkenyl group can be optionally substituted.

“Alkoxy” refers to a radical of the formula —OR where R is an alkyl, alkenyl or alkynyl radical as defined above containing one to twelve carbon atoms. Unless stated otherwise specifically in the specification, an alkoxy group can be optionally substituted.

“Alkylamino” refers to a radical of the formula —NHRₐ or —NRₐRₐ where each Rₐ is, independently, an alkyl, alkenyl or alkynyl radical as defined above containing one to twelve carbon atoms. Unless stated otherwise specifically in the specification, an alkylamino group can be optionally substituted.

“Aryl” refers to a hydrocarbon ring system radical comprising hydrogen, 6 to 18 carbon atoms and at least one aromatic ring. For purposes of this invention, the aryl radical can be a monocyclic, bicyclic, tricyclic or tetracyclic ring system, which can include fused or bridged ring systems. Aryl radicals include, but are not limited to, aryl radicals derived from aceanthrylene, acenaphthylene, acephenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, the term “aryl” is meant to include aryl radicals that are optionally substituted.

“Aralkyl”, “arylalkyl” or “alkyene-aryl” refers to a radical of the formula —R_{b}—R_{c} where R_{b} is an alkylene group as defined above and R_{c} is one or more aryl radicals as defined above, for example, benzyl, diphenylmethyl and the like. Unless stated otherwise specifically in the specification, an aralkyl group can be optionally substituted.

“Carbocyclyl,” “carbocyclic ring” or “carbocycle” refers to a non-aromatic ring structure, wherein the atoms which form the ring are each carbon. Carbocyclic rings can comprise from 3 to 20 carbon atoms in the ring. Carbocyclic rings include cycloalkyl, cycloalkenyl and cycloalkynyl as defined herein. Unless stated otherwise specifically in the specification, a carbocyclyl group can be optionally substituted.

“Cycloalkyl” refers to a stable non-aromatic monocyclic or polycyclic fully saturated hydrocarbon radical consisting solely of carbon and hydrogen atoms, which can include fused or bridged ring systems, having from three to twenty carbon atoms, e.g., having from three to ten carbon atoms, and which is attached to the rest of the molecule by a single bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, 7,7-dimethyl-bicyclo[2.2.1]heptanyl, and the like. Unless otherwise stated specifically in the specification, a cycloalkyl group can be optionally substituted.

“Cycloalkenyl” refers to a stable non-aromatic monocyclic or polycyclic hydrocarbon radical consisting solely of carbon and hydrogen atoms, having one or more carbon-carbon double bonds, which can include fused or bridged ring systems, having from three to twenty carbon atoms, e.g., having from three to ten carbon atoms, and which is attached to the rest of the molecule by a single bond. Monocyclic cycloalkenyl radicals include, for example, cyclopentenyl, cyclohexenyl, cycloheptenyl, cycloctenyl, and the like. Polycyclic cycloalkenyl radicals include, for example, bicyclo[2.2.1]hept-2-enyl and the like. Unless otherwise stated specifically in the specification, a cycloalkenyl group can be optionally substituted.

“Cycloalkynyl” refers to a stable non-aromatic monocyclic or polycyclic hydrocarbon radical consisting solely of carbon and hydrogen atoms, having one or more carbon-carbon triple bonds, which can include fused or bridged ring systems, having from three to twenty carbon atoms, e.g., having from three to ten carbon atoms, and which is attached to the rest of the molecule by a single bond. Monocyclic cycloalkynyl radicals include, for example, cycloheptynyl, cyclooctynyl, and the like. Unless otherwise stated specifically in the specification, a cycloalkynyl group can be optionally substituted.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. Unless stated otherwise specifically in the specification, a haloalkyl group can be optionally substituted.

“Haloalkenyl” refers to an alkenyl radical, as defined above, that is substituted by one or more halo radicals, as defined above, e.g., 1-fluoropropenyl, 1,1-difluorobutenyl, and the like. Unless stated otherwise specifically in the specification, a haloalkenyl group can be optionally substituted.

“Haloalkynyl” refers to an alkynyl radical, as defined above, that is substituted by one or more halo radicals, as defined above, e.g., 1-fluoropropynyl, 1-fluorobutynyl, and the like. Unless stated otherwise specifically in the specification, a haloalkynyl group can be optionally substituted.

“Heterocyclyl” “heterocyclic ring” or “heterocycle” refers to a stable 3- to 20-membered non-aromatic radical which consists of two to twelve carbon atoms and from one to six heteroatoms selected from nitrogen, oxygen, sulfur, or silicon. Unless stated otherwise specifically in the specification, the heterocyclyl radical can be a monocyclic, bicyclic, tricyclic or tetracyclic ring system, which can include fused or bridged ring systems; and the nitrogen, carbon or sulfur atoms in the heterocyclyl radical can be optionally oxidized; the nitrogen atom can be optionally quaternized; and the heterocyclyl radical can be partially or fully saturated. Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, a heterocyclyl group can be optionally substituted.

“Heterocyclylalkyl” or “alkylene-heterocyclyl” refers to a radical of the formula —R_{b}—R_{c} where R_{b} is an alkylene group as defined above and R_{c} is a heterocyclyl radical as defined above. Unless stated otherwise specifically in the specification, a heterocycloalkyl group can be optionally substituted.

“N-heterocyclyl” refers to a heterocyclyl radical as defined above containing at least one nitrogen and where the point of attachment of the heterocyclyl radical to the rest of the molecule is through a nitrogen atom in the heterocyclyl radical. Unless stated otherwise specifically in the specification, a N-heterocyclyl group can be optionally substituted.

“Heteroaryl” refers to a 5- to 20-membered ring system radical comprising hydrogen atoms, one to thirteen carbon atoms, one to six heteroatoms selected from nitrogen, oxygen and sulfur, and at least one aromatic ring comprising at least one heteroatom selected from nitrogen, oxygen and sulfur. For purposes of this invention, the heteroaryl radical can be a monocyclic, bicyclic, tricyclic or tetracyclic ring system, which can include fused or bridged ring systems; and the nitrogen, carbon or sulfur atoms in the heteroaryl radical can be optionally oxidized; the nitrogen atom can be optionally quaternized. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophene), benzotriazolyl, benzo[4,6]imidazo[1,2-a]pyridinyl, carbazolyl, cinnolinyl, dibenzofuranyl, dibenzothiophene, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophene (i.e. thienyl). Unless stated otherwise specifically in the specification, a heteroaryl group can be optionally substituted.

“N-heteroaryl” refers to a heteroaryl radical as defined above containing at least one nitrogen and where the point of attachment of the heteroaryl radical to the rest of the molecule is through a nitrogen atom in the heteroaryl radical. Unless stated otherwise specifically in the specification, an N-heteroaryl group can be optionally substituted.

“Heteroarylalkyl” or “alkylene-heteroaryl” refers to a radical of the formula —R_{b}—R_{f} where R_{b} is an alkylene chain as defined above and R_{f} is a heteroaryl radical as defined above. Unless stated otherwise specifically in the specification, a heteroarylalkyl group can be optionally substituted.

“Thioalkyl” refers to a radical of the formula —SR where R. is an alkyl, alkenyl, or alkynyl radical as defined above containing one to twelve carbon atoms. Unless stated otherwise specifically in the specification, a thioalkyl group can be optionally substituted.

The term “substituted” used herein means any of the above groups (e.g., alkyl, alkylene, alkenyl, alkenylene, alkynyl, alkynylene, alkoxy, alkylamino, thioalkyl, aryl, aralkyl, carbocyclyl, cycloalkyl, cycloalkenyl, cycloalkynyl, cycloalkylalkyl, haloalkyl, heterocyclyl, N-heterocyclyl, heterocyclylalkyl, heteroaryl, N-heteroaryl and/or heteroarylalkyl) wherein at least one hydrogen atom is replaced by a bond to a non-hydrogen atoms such as, but not limited to: a halogen atom such as F, Cl, Br, and I; an oxygen atom in groups such as hydroxyl groups, alkoxy groups, and ester groups; a sulfur atom in groups such as thiol groups, thioalkyl groups, sulfone groups, sulfonyl groups, and sulfoxide groups; a nitrogen atom in groups such as amines, amides, alkylamines, dialkylamines, arylamines, alkylarylamines, diarylamines, N-oxides, imides, and enamines; a silicon atom in groups such as trialkylsilyl groups, dialkylarylsilyl groups, alkyldiarylsilyl groups, and triarylsilyl groups; and other heteroatoms in various other groups.

“Substituted” also means any of the above groups in which one or more hydrogen atoms are replaced by a higher-order bond (e.g., a double- or triple-bond) to a heteroatom such as oxygen in oxo, carbonyl, carboxyl, and ester groups; and nitrogen in groups such as imines, oximes, hydrazones, and nitriles. For example, “substituted” includes any of the above groups in which one or more hydrogen atoms are replaced with —NR_{g}Rₕ, —NR_{g}C(═O)Rₕ, —NR_{g}C(═O)NR_{g}Rₕ, —NR_{g}C(═O)ORₕ, —NR_{g}SO₂Rₕ, —OC(═O)NR_{g}Rₕ, —OR_{g}, —SR_{g}, —SOR_{g}, —SO₂R_{g}, —OSO₂R_{g}, —SO₂OR_{g}, ═NSO₂R_{g}, and —SO₂NR_{g}Rₕ, “Substituted also means any of the above groups in which one or more hydrogen atoms are replaced with —C(═O)R_{g}, —C(═O)OR_{g}, —C(═O)NR_{g}Rₕ, —CH₂SO₂R_{g}, —CH₂SO₂NR_{g}Rₕ. In the foregoing, R_{g} and Rₕ are the same or different and independently hydrogen, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl.

“Substituted” further includes any of the above groups in which one or more hydrogen atoms are replaced by a bond to an amino, cyano, hydroxyl, imino, nitro, oxo, thioxo, halo, alkyl, alkenyl, alkynyl, alkoxy, alkylamino, thioalkyl, aryl, aralkyl, cycloalkyl, cycloalkenyl, cycloalkynyl, cycloalkylalkyl, haloalkyl, haloalkenyl, haloalkynyl, heterocyclyl, N-heterocyclyl, heterocyclylalkyl, heteroaryl, N-heteroaryl and/or heteroarylalkyl group.

In this specification, unless stated otherwise, the term “pharmaceutically acceptable” is used to characterize a moiety (e.g., a salt, dosage form, or excipient) as being appropriate for therapeutic use. In general, a pharmaceutically acceptable moiety has one or more benefits that outweigh any deleterious effect that the moiety may have. Deleterious effects may include, for example, excessive toxicity, irritation, allergic response, and other problems and complications.

The term “pharmaceutically acceptable salt” includes both acid and base addition salts. Pharmaceutically acceptable salts include those obtained by reacting the active compound functioning as a base, with an inorganic or organic acid to form a salt, for example, salts of hydrochloric acid, sulfuric acid, phosphoric acid, methanesulfonic acid, camphorsulfonic acid, oxalic acid, maleic acid, succinic acid, citric acid, formic acid, hydrobromic acid, benzoic acid, tartaric acid, fumaric acid, salicylic acid, mandelic acid, carbonic acid, etc. Those skilled in the art will further recognize that acid addition salts may be prepared by reaction of the compounds with the appropriate inorganic or organic acid via any of a number of known methods.

The compounds of the disclosure, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present disclosure is meant to include all such possible isomers, as well as their racemic and optically pure forms whether or not they are specifically depicted herein. Optically active (+) and (−), (R)- and (S)-, or (D)- and (L)-isomers can be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centers of geometric asymmetry, and unless specified otherwise, it is intended that the compounds include both E and Z geometric isomers. Likewise, all tautomeric forms are also intended to be included.

### Compounds

In embodiments, the disclosure provides a compound of Formula (I)

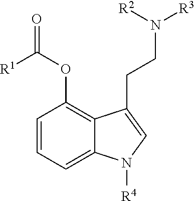

(I), or a pharmaceutically acceptable salt thereof.

In embodiments, R¹ is NR⁵R⁶, alkyl, haloalkyl, alkenyl, alkylene-NR⁵R⁶, alkyene-C(═O)O-alkyl, alkyene-C(═O)O-heteroaryl wherein the heteroaryl is optionally substituted with alkyl or alkylene-NR⁵R⁶, alkylene-O-alkyl, alkylene-OC(═O)alkyl, alkylene-O-haloalkyl, alkylene-O-alkylene-O-alkyl, alkylene-C(═O)NR⁵R⁶, alkylene-aryl optionally substituted with 1, 2, 3, or 4 R⁷, alkylene-heteroaryl, alkylene-carbocyclyl, alkylene-heterocyclyl, aryl optionally substituted with 1, 2, 3, or 4 R⁷, heteroaryl optionally substituted with 1, 2, 3, or 4 R⁷, O-alkyl, O-alkylene-heterocyclyl, O-heterocyclyl, O-alkylene-carbocyclyl, O-aryl optionally substituted with 1, 2, 3, or 4 R⁷, O-alkylene-heteroaryl, or carbocyclyl optionally substituted with Si(alkyl)₃.

In embodiments, R² and R³ are independently alkyl.

In embodiments, R⁴ is H or C(═O)Oalkyl.

In embodiments, R and R⁶ are independently H, OH, alkyl, C(═O)alkyl, or C(═O)Oalkyl.

In embodiments, R⁷ is OH, halo, alky, NR⁵R⁶, OC(═O)alkyl, or O-alkyl.

In embodiments, the disclosure provides a compound of Formula (II)

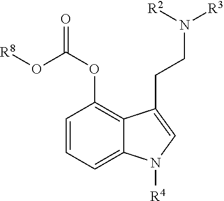

(II), or a pharmaceutically acceptable salt thereof,

In embodiments, R² and R³ are independently alkyl.

In embodiments, R⁴ is H or C(═O)Oalkyl.

In embodiments, R⁸ is alkylene-carbocyclyl, alkylene-heterocyclyl, alkylene-heterocyclyl substituted with 1, 2, 3, or 4 R⁷, alkylene-C(═O)NR⁵R⁶, alkylene-C(═O)aryl, alkylene-OC(═O)alkyl, alkylene-N+(C₁₋₆ alkyl)₃, alkylene-NR⁵R⁶, alkylene-S(O)₂(alkyl), alkylene-S(O)₂(NR⁵R⁶), alkylene-Si(alkyl)₃, (CH₂CH₂O)ₘalkyl, heterocyclyl, or aryl substituted with 1, 2, 3, or 4 R⁷.

In embodiments, R⁵ and R⁶ are independently H, alkyl, C(═O)alkyl, alkylene-aryl, or S(O)₂alkyl.

In embodiments, R⁷ is halo, alky, or O-alkyl.

In embodiments, m is 2, 3, 4, 5, 6, 7, or 8.

In some embodiments of compound of Formula (III)

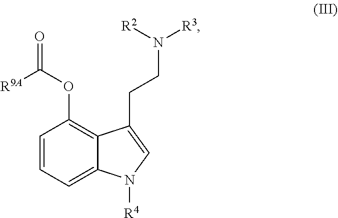

or a pharmaceutically acceptable salt thereof,
R^{9A} is branch C₁₋₆ alkyl substituted with —O-aryl or —OC(═O)C₁₋₆alkyl, aryl optionally substituted with 1-4 R⁷, 5-10 membered heteroaryl optionally substituted with OH, 3-10 membered heterocyclyl optionally substituted with C(═O)Oalkyl or haloalkyl, 3-8 membered carbocyclyl, 3-8 membered carbocyclyl substituted with aryl or Si(alkyl)₃, —CH(R^{A})—C₁₋₆ alkyl, —CH(R^{A})NR⁵R⁶, —CH(R^{A})—O-aryl, —CH(R^{A})-aryl, —CH(R^{A})-5-10 membered heteroaryl, C(R^{A})₂—Si(C₁₋₃ alkyl)₃,
each R^{A} is independently H, OH, halo, unsubstituted C₁₋₆ alky, or C₁₋₆ alkylene-OH;
R² and R³ are independently C₁₋₄ alkyl;
R⁴ is H or C(═O)OC₁₋₄alkyl;
R⁵ and R⁶ are independently H, C₁₋₆ alkyl, C(═O)C₁₋₆ alkyl, C(═O)aryl, C(═O)O C₁₋₆alkyl, S(O)₂—C₁₋₆ alkyl, C₁₋₆ alkylene-3-10-membered heterocyclyl; and
R⁷ is OH, halo, alky, O-alkyl, OC(═O)C₁₋₆alky, Si(C₁₋₃alkyl)₃, aryl, or 3-10 membered heterocycle;
provided that R^{9A} is not

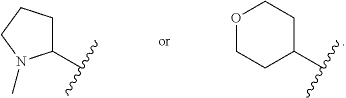

In embodiments, the disclosure provides a compound of Formula (IV)

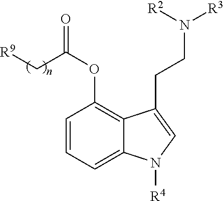

(IV), or a pharmaceutically acceptable salt thereof,

In embodiments, R¹ is branched alkyl, haloalkyl, alkylene-OH, NR⁵R⁶, S(O)₂NR⁵R⁶, S(O)₂alkyl, alkylene-NR⁵R⁶, C(═O)O-alkyl, C(═O)NR⁵R⁶, C(═O)O-heteroaryl optionally substituted with alkyl or alkylene-NR⁵R⁶, alkenylene-NR⁵R⁶, alkenylene-aryl, alkenylene(OH)(aryl), alkylene-OC(═O)alky, alkylene-O-aryl optionally substituted with 1, 2, 3, or 4 R⁷, O-alkyl, O-haloalkyl, O-alkenylene-NR⁵R⁶, O-alkylene-O-alkyl, O-aryl, OC(═O)alkyl, OC(═O)haloalkyl, Si(alkyl)₃, haloalkylSi(alkyl)₃, aryl optionally substituted with 1, 2, 3, or 4 R⁷, heteroaryl, heterocyclyl optionally substituted with C(═O)Oalkyl, carbocyclyl, carbocyclyl substituted with aryl.

In embodiments, R⁴ is H or C(═O)Oalkyl.

In embodiments, R⁵ and R⁶ are independently H, alkyl, C(═O)alkyl, C(═O)aryl, C(═O)Oalkyl, S(O)₂alkyl, alkylene-heterocyclyl.

In embodiments, R⁷ is OH, halo, alky, O-alkyl, OC(═O)alky, or aryl.

In embodiments, n is 1, 2, 3, 4, 5, or 6.

In embodiments, when R¹ is NR⁵R⁶, n is not 1 or R⁵ and R⁶ are both not H. In embodiments, when R⁹ is aryl, n is not 1.

In embodiments, the disclosure provides a compound of Formula (V)

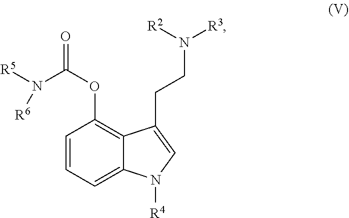

or a pharmaceutically acceptable salt thereof,
In embodiments, R² and R³ are independently alkyl.
In embodiments, R⁴ is H or C(═O)Oalkyl.
In embodiments, R⁵ and R⁶ are independently H, OH, alkyl, haloalkyl, C(═O)alkyl, C(═O)Oalkyl, Oalkyl, or aryl optionally substituted with C(═O)NH(alkyl). In embodiments, R⁵ and R⁶ together with the atoms to which they are attach form heterocyclyl.

In embodiments, the disclosure provides a compound of Formula (VI)

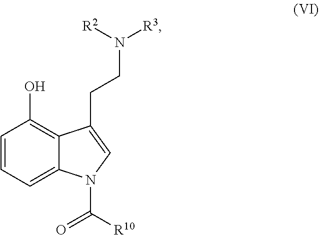

or a pharmaceutically acceptable salt thereof,

In embodiments, R² and R³ are independently alkyl.

In embodiments, R¹⁰ is alkyl, alkylene-C(═O)Oalkyl, O-alkylene-aryl optionally substituted with 1, 2, 3, or 4 R⁷.

In embodiments, R⁷ is OH, halo, alky, O-alkyl, OC(O)alky, alkylene-O—C(═O)alkyl.

In some embodiments, this disclosure provides a compound of Formula (I)

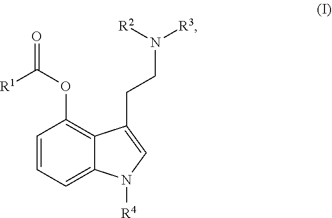

or a pharmaceutically acceptable salt thereof, wherein:
R² and R³ are independently alkyl;
R⁴ is H or C(═O)Oalkyl;
R¹ is defined according to i, ii, iii, or iv.

In embodiments of Formula (I) wherein R¹ is defined according to i, R¹ is —O—R⁸, wherein
R⁸ is alkylene-carbocyclyl, alkylene-heterocyclyl wherein the heterocyclyl is substituted with 1-4 R⁷, alkylene-aryl wherein the aryl is substituted with 1-4 R⁷, alkylene-heteroaryl, alkylene-heteroaryl wherein the heteroaryl is substituted with 1-4 R⁷, alkylene-C(═O)NR⁵R⁶, alkylene-C(═O)aryl, alkylene-OC(═O)alkyl, alkylene-OH,-alkylene-N⁺(C₁₋₆ alkyl)₃, alkylene-NR⁵R⁶, alkylene-S(O)₂(alkyl), alkylene-S(O)₂(NR⁵R⁶), alkylene-Si(alkyl)₃, (CH₂CH₂O)ₘalkyl, —C(═O)alkyl, or aryl substituted with 1-4 R⁷;
R⁵ and R⁶ are independently H, alkyl, C(═O)alkyl, alkylene-aryl, or S(O)₂alkyl;
R⁷ is OH, halo, alkyl, haloalkyl, O-alkyl, OC(═O)alky, Si(alkyl)₃, aryl, —NH(C═O)alkyl, —N(alkyl)₂, or heterocyclyl;
m is 2, 3, 4, 5, 6, 7, or 8; or

In embodiments of Formula (I) wherein R¹ is defined according to ii, R¹ is R^{9A}, wherein
R^{9A} is branch alkyl substituted with or —OC(═O)alkyl or —O-aryl optionally substituted with 1-4 R⁷, alkenyl substituted with aryl, aryl optionally substituted with 1-4 R⁷, heteroaryl optionally substituted with 1-4 R⁷, heterocyclyl optionally substituted with 1-4 R⁷, carbocyclyl optionally substituted with 1-4 R⁷, —C(R^{A})₂-alkyl, —C(R^{A})₂NR⁵R⁶; —C(R⁴)₂—O-aryl, —C(R^{A})₂-aryl, —C(R^{A})₂-heteroaryl, or C(R^{A})₂—Si(alkyl)₃,
each R^{A} is independently H, OH, halo, unsubstituted alky, or alkylene-OH, provided that at least one R^{A} is not H, and when R^{9A} is —C(R^{A})₂-alkyl then the two R^{A} cannot both be CH₃;
R³ and R⁶ are independently H, alkyl, C(═O)alkyl, C(═O)aryl, C(═O)Oalkyl, S(O)₂alkyl, alkylene-heterocyclyl;
R⁷ is OH, halo, alkyl, haloalkyl, O-alkyl, OC(═O)alky, Si(alkyl)₃, aryl, —NH(C═O)alkyl, —N(alkyl)₂, or heterocyclyl; provided that R^{9A} is not

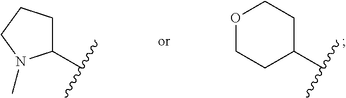

or

In embodiments of Formula (I) wherein R¹ is defined according to iii, R¹ is —(CH₂)ₙ—R^{9B}, wherein
n is 1, 2, 3, 4, 5, or 6;
R^{9B} is haloalkyl, alkylene-OH, alkenyl, NR⁵R⁶, S(O)₂NR⁵R⁶, S(O)₂alkyl, C(═O)Oalkyl, C(═O)NR⁵R⁶, C(═O)O-heteroaryl optionally substituted with alkyl or alkylene-NR⁵R⁶, O-alkyl, O-haloalkyl, O-alkenylene-NR⁵R⁶, O-alkylene-O-alkyl, O-aryl, OC(═O)alkyl, OC(═O)haloalkyl, Si(alkyl)₃, aryl optionally substituted with 1-4 R⁷, heteroaryl, heterocyclyl optionally substituted with C(═O)Oalkyl, carbocyclyl, carbocyclyl substituted with aryl; provided that when R^{9B} is C(═O)Oalkyl, then n is less than 5; further provided that when R^{9B} is NR⁵R⁶, n is not 1 or R⁵ and R⁶ are both not H, and when R^{9A} is aryl, n is not 1; further provided that R^{9B} is not

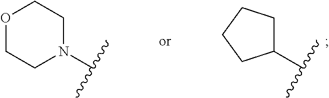 [SMILES: *N1CCOCC1 ; *C1CCCC1]

R⁷ is OH, halo, alkyl, haloalkyl, O-alkyl, OC(═O)alky, Si(alkyl)₃, aryl, —NH(C═O)alkyl, —N(alkyl)₂, or heterocyclyl; or

In embodiments of Formula (I) wherein R¹ is defined according to iv, R¹ is —NR^{5A}R^{6A}, wherein
R^{5A} and R^{6A} are independently H, OH, unsubstituted C₁₋₆ alkyl, haloalkyl, C(═O)alkyl, C(═O)Oalkyl, O-alkyl, or aryl substituted with C(═O)NH(alkyl), provided that R^{5A} and R^{6A} cannot both be H, and further provided that when one of R^{5A} and R^{6A} is CH₃, then the other cannot be H, CH₃ or CH₂CH₃;
or R^{5A} and R^{6A} together with the atoms to which they are attached form a heterocyclyl.

In some embodiments, this disclosure provides a compound of Formula (II)

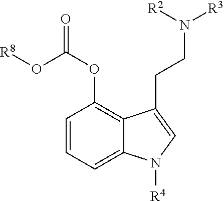

(II), or a pharmaceutically acceptable salt thereof, wherein:
R⁸ is alkylene-heterocyclyl, alkylene-heterocyclyl wherein the heterocyclyl is substituted with 1-4 R⁷, alkylene-aryl wherein the aryl is substituted with 1-4 R⁷, alkylene-heteroaryl, alkylene-heteroaryl wherein the heteroaryl is substituted with 1-4 R⁷, alkylene-C(═O)NR⁵R⁶, alkylene-C(═O)aryl, alkylene-OC(═O)alkyl, alkylene-OH,-alkylene-N⁺(C₁₋₆ alkyl)₃, alkylene-NR⁵R⁶, alkylene-S(O)₂(alkyl), alkylene-S(O)₂(NR⁵R⁶), alkylene-Si(alkyl)₃, (CH₂CH₂O)ₘalkyl, or aryl substituted with 1-4 R⁷;
R² and R³ are independently alkyl;
R⁴ is H or C(═O)Oalkyl;
R⁵ and R⁶ are independently H, alkyl, C(═O)alkyl, alkylene-aryl, or S(O)₂alkyl;
R⁷ is halo, alky, OH, or O-alkyl; and
m is 2, 3, 4, 5, 6, 7, or 8.

In some embodiments of compound of Formula (II)

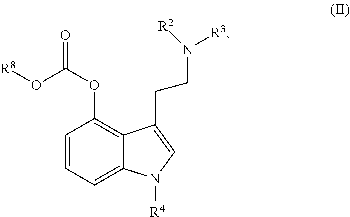

(II), or a pharmaceutically acceptable salt thereof,
R⁸ is unsubstituted C₁₋₆ alkylene-C₃₋₁₀ carbocyclyl, C₁₋₆ alkylene-3-10 membered heterocyclyl wherein the heterocycle is substituted with 1-4 R⁷, C₁₋₆ alkylene-aryl wherein the aryl is substituted with 1-4 R⁷, C₁₋₆ alkylene-5-10 membered heteroaryl, C₁₋₆alkylene-5-10 membered heteroaryl wherein the heteroaryl is substituted with 1-4 R⁷, C₁₋₆ alkylene-C(═O)NR⁵R⁶, C₁₋₆ alkylene-C(═O)aryl, C₁₋₆ alkylene-OC(═O)—C₁₋₆ alkyl, C₁₋₆ alkylene-OH, C₁₋₆ alkylene-N+(C₁₋₆ alkyl)₃, C₁₋₆ alkylene-NR⁵R⁶, C₁₋₆ alkylene-S(O)₂(C₁₋₆ alkyl), C₁₋₆ alkylene-S(O)₂(NR⁵R⁶), alkylene-Si(C₁₋₃alkyl)₃, (CH₂CH₂O)ₘalkyl, —C(═O)—C₁₋₆ alkyl, or aryl substituted with 1-4 R⁷;
R² and R³ are independently C₁₋₃ alkyl;
R⁴ is H or C(═O)O C₁₋₆ alkyl;
R⁵ and R⁶ are independently H, C₁₋₆ alkyl, C(═O) C₁₋₆alkyl, C₁₋₆ alkylene-aryl, or S(O)₂—C₁₋₆ alkyl;
R⁷ is halo, C₁₋₆ alky, OH, or O—C₁₋₆ alkyl; and
m is 2, 3, 4, 5, 6, 7, or 8.

In some embodiments of compound of formula (II),
R² and R³ are independently C₁₋₃ alkyl:
R⁴ is H or C(═O)O—C₁₋₆alkyl;
R⁸ is C₁₋₆ alkylene-C₃₋₁₀ carbocyclyl, or aryl substituted with 1, 2, 3, or 4 R⁷; and
R⁷ is halo, C₁₋₆ alky, O—C₁₋₆ alkyl.

In some embodiments, this disclosure provides a compound of Formula (III)

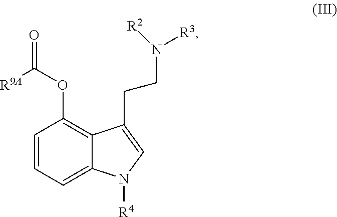

or a pharmaceutically acceptable salt thereof, wherein
R^{9A} is branch alkyl substituted with or —OC(═O)alkyl or —O-aryl optionally substituted with 1-4 R⁷, aryl optionally substituted with 1-4 R⁷, heteroaryl optionally substituted with 1-4 R⁷, heterocyclyl optionally substituted with 1-4 R⁷, carbocyclyl optionally substituted with 1-4 R⁷, —C(R^{A})₂-alkyl, —C(R^{A})₂NR⁵R⁶; —C(R^{A})₂—O-aryl, —C(R^{A})₂-aryl, —C(R^{A})₂-heteroaryl, or C(R^{A})₂—Si(alkyl)₃,
each R^{A} is independently H, OH, halo, unsubstituted alkyl, or alkylene-OH, provided that at least one R^{A} is not H, and when R^{9A} is —C(R^{A})₂-alkyl then the two R^{A} cannot both be CH₃;
R² and R³ are independently alkyl;
R⁴ is H or C(═O)Oalkyl:
R⁵ and R⁶ are independently H, alkyl, C(═O)alkyl, C(═O)aryl, C(═O)Oalkyl, S(O)₂alkyl, alkylene-heterocyclyl;
R⁷ is OH, halo, alkyl, haloalkyl, O-alkyl, OC(═O)alky, Si(alkyl)₃, aryl, —NH(C═O)alkyl, —N(alkyl)₂, or heterocyclyl;
provided that R^{9A} is not

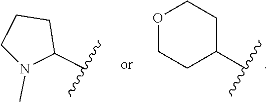

In some embodiments of the compound of Formula (III)
R^{9A} is branch alkyl substituted with —O-aryl or —OC(═O)alkyl, aryl optionally substituted with 1-3 R⁷, heteroaryl optionally substituted with OH, heterocyclyl optionally substituted with C(═O)Oalkyl, haloalkyl, carbocyclyl optionally substituted with aryl or Si(alkyl)₃, —CH(R^{A})-alkyl, —CH(R^{A})NR⁵R⁶, —CH(R^{A})—O-aryl, —CH(R^{A})-aryl, —CH(R^{A})-heteroaryl, C(R^{A})₂—Si(alkyl)₃;
each one of R^{A} is independently H, OH, halo, unsubstituted alky, or alkylene-OH;
R² and R³ are independently C₁₋₄ alkyl;
R⁴ is H or C(═O)OC₁₋₄alkyl;
R⁵ and R⁶ are independently H, alkyl, C(═O)alkyl, C(═O)aryl, C(═O)Oalkyl, S(O)₂alkyl, alkylene-heterocyclyl;
R⁷ is OH, halo, alky, haloalkyl O-alkyl, OC═O alky, Si(alkyl)₃, aryl, —N(alkyl)₂ or heterocycle; provided that R^{9A} is not

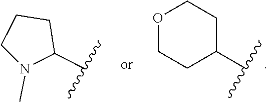

In some embodiments of compound of Formula (III)

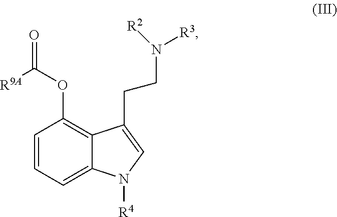

or a pharmaceutically acceptable salt thereof,
R^{9A} is branch C₁₋₆ alkyl substituted with —O-aryl or —OC(═O)C₁₋₆alkyl, aryl optionally substituted with 1-4 R⁷, 5-10 membered heteroaryl optionally substituted with OH, 3-10 membered heterocyclyl optionally substituted with C(═O)Oalkyl or haloalkyl, 3-8 membered carbocyclyl, 3-8 membered carbocyclyl substituted with aryl or Si(alkyl)₃, —CH(R^{A})—C₁₋₆ alkyl, —CH(R^{A})NR⁵R⁶, —CH(R^{A})—O-aryl, —CH(R^{A})-aryl, —CH(R^{A})-5-10 membered heteroaryl, C(R^{A})₂—Si(C₁₋₃ alkyl)₃,
each R^{A} is independently H, OH, halo, unsubstituted C₁₋₆ alkyl, or C₁₋₆ alkylene-OH;
R² and R³ are independently C₁₋₄ alkyl;
R⁴ is H or C(═O)OC₁₋₄alkyl;
R⁵ and R⁶ are independently H, C₁₋₆ alkyl, C(═O)C₁₋₆ alkyl, C(═O)aryl, C(═O)O C₁₋₆alkyl, S(O)₂—C₁₋₆alkyl, C₁₋₆alkylene-3-10-membered heterocyclyl; and
R⁷ is OH, halo, alky, O-alkyl, OC(═O)C₁₋₆alky, Si(C₁₋₃alkyl)₃, aryl, or 3-10 membered heterocycle;
provided that R^{9A} is not

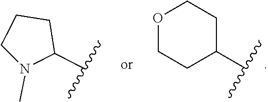

In some embodiments, this disclosure provides a compound of Formula (IV)

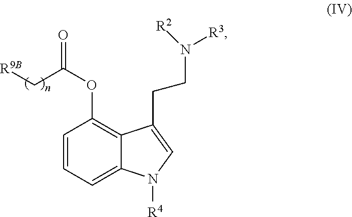

or a pharmaceutically acceptable salt thereof, wherein
n is 1, 2, 3, 4, 5, or 6;
R^{9B} is haloalkyl, alkylene-OH, NR⁵R⁶, S(O)₂NR⁵R⁶, S(O)₂alkyl, C(═O)Oalkyl, C(═O)NR⁵R⁶, C(═O)O-heteroaryl optionally substituted with alkyl or alkylene-NR⁵R⁶, O-alkyl, O-haloalkyl, O-alkenylene-NR⁵R⁶, O-alkylene-O-alkyl, O-aryl, OC(═O)alkyl, OC(═O)haloalkyl, Si(alkyl)₃, aryl optionally substituted with 1-4 R⁷, heteroaryl, heterocyclyl optionally substituted with C(═O)Oalkyl, carbocyclyl, carbocyclyl substituted with aryl; provided that when R^{9B} is C(═O)Oalkyl, then n is less than 5;
R² and R³ are independently alkyl;
R⁴ is H or C(═O)Oalkyl;
R⁵ and R⁶ are independently H, alkyl, C(═O)alkyl, C(═O)aryl, C(═O)Oalkyl, S(O)₂alkyl, alkylene-heterocyclyl;
R⁷ is OH, halo, alkyl, O-alkyl, OC(═O)alky, Si(alkyl)₃, aryl, heterocyclyl; and provided that when R⁹ is NR⁵R⁶, n is not 1 or R⁵ and R⁶ are both not H, and when R⁹ is aryl, n is not 1; further provided that, and R^{9B} is not

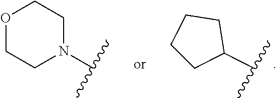 [SMILES: *N1CCOCC1 ; *C1CCCC1]

In some embodiments of the compound of Formula (IV)

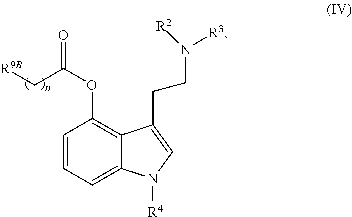

or a pharmaceutically acceptable salt thereof,
n is 1, 2, 3, 4, 5, or 6,
R^{9B} is C₁₋₆ haloalkyl, C₁₋₆ alkylene-OH, NR⁵R⁶, S(O)₂NR⁵R⁶, S(O)₂—C₁₋₆ alkyl, C(═O)NR⁵R⁶, C(═O)O-5-10 membered heteroaryl optionally substituted with C₁₋₆ alkyl or C₁₋₆ alkylene-NR⁵R⁶, O—C₁₋₆ alkyl, O—C₁₋₆haloalkyl, O—C₁₋₆ alkenylene-NR⁵R⁶, O—C₁₋₆ alkylene-O—C₁₋₆alkyl, O-aryl, OC(═O)—C₁₋₆alkyl, OC(═O) C₁₋₆haloalkyl, Si(C₁₋₃alkyl)₃, aryl optionally substituted with 1-4 R⁷, 5-10 membered heteroaryl, 3-10 membered heterocyclyl optionally substituted with C(═O)OC₁₋₆ alkyl, 3-10 membered carbocyclyl, 3-10 membered carbocyclyl substituted with aryl;
R² and R³ are independently C₁₋₃ alkyl;
R⁴ is H or C(═O)O C₁₋₆ alkyl;
R⁵ and R⁶ are independently H, C₁₋₆alkyl, C(═O) C₁₋₆alkyl, C(═O)aryl, C(═O)O C₁₋₆ alkyl, S(O)₂—C₁₋₆alkyl, C₁₋₆alkylene-3-10 membered heterocyclyl;
R⁷ is OH, halo, C₁₋₆ alky, O—C₁₋₆ alkyl, OC(═O)C₁₋₆ alky, Si(C₁₋₃alkyl)₃, aryl, or heterocycle; and
provided that when R⁹ is NR⁵R⁶, n is not 1 or R⁵ and R⁶ are both not H; and when R⁹ is aryl, n is not 1; further provided that R^{9B} is not

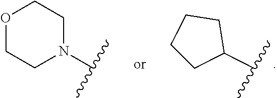 [SMILES: *N1CCOCC1 ; *C1CCCC1]

In some embodiments, this disclosure provides a compound of Formula (V)

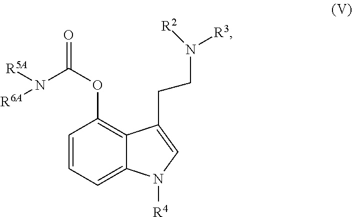

or a pharmaceutically acceptable salt thereof, wherein:
R² and R³ are independently alkyl;
R⁴ is H or C(═O)Oalkyl;
R^{5A} and R^{6A} are independently H, OH, unsubstituted C₁₋₆ alkyl, haloalkyl, C(═O)alkyl, C(═O)Oalkyl, O-alkyl, or aryl substituted with C(═O)NH(alkyl), provided that R^{5A} and R^{6A} cannot both be H, and further provided that when one of R^{5A} and R^{6A} is CH₃, the other cannot be H, CH₃ or CH₂CH₃;
or R^{5A} and R^{6A} together with the atoms to which they are attached form a heterocyclyl.

In some embodiments of compound of formula (V),

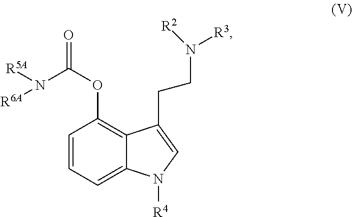

or a pharmaceutically acceptable salt thereof,
R² and R³ are independently C₁₋₃ alkyl;
R⁴ is H or C(═O)O—C₁₋₆ alkyl;
R^{5A} and R^{6A} are independently H, OH, unsubstituted C₃₋₆ alkyl, haloalkyl, C(═O) C₁₋₆ alkyl, C(═O)OC₁₋₆ alkyl, O—C₁₋₆alkyl, or aryl substituted with C(═O)NH(C₁₋₆ alkyl), provided that R^{5A} and R^{6A} cannot both be H, and further provided that when one of R^{1A} and R^{6A} is CH₃, the other cannot be H, CH₃ or CH₂CH₃,
or R^{5A} and R^{6A} together with the atoms to which they are attached form a 3-10 membered heterocyclyl.

In some embodiments, this disclosure provides a compound of Formula (VI)

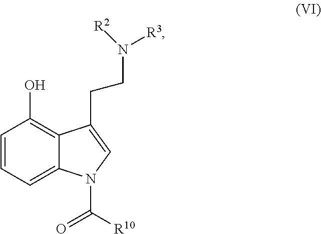

or a pharmaceutically acceptable salt thereof, wherein:
R² and R³ are independently alkyl;
R¹⁰ is O-linear alkyl, alkylene-C(═O)Oalkyl, O-alkylene-aryl optionally substituted with 1, 2, 3, or 4 R⁷; and
R⁷ is OH, halo, alky, O-alkyl, OC(═O)alky, alkylene-O—C(═O)alkyl.

In some embodiment of compounds of Formula (VI)

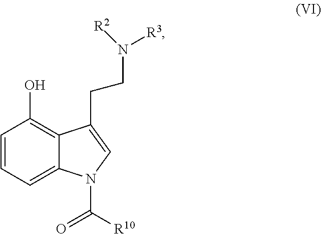

or a pharmaceutically acceptable salt thereof,
R² and R³ are independently C₁₋₃ alkyl,
R¹⁰ is O-linear C₁₋₆ alkyl, C₁₋₆ alkylene-C(═O)OC₁₋₆ alkyl, O—C₁₋₆alkylene-aryl optionally substituted with 1, 2, 3, or 4 R⁷; and
R⁷ is OH, halo, C₁₋₆ alky, O-alkyl, OC(═O)—C₁₋₆alky, alkylene-O—C(═O)C₁₋₆ alkyl.

In some embodiments, this disclosure provides a compound selected from any of the compounds in Table 1, Table 2, Table 3, Table 4 or Table 5, or a pharmaceutical salt or deuterated form thereof. In some embodiments, this disclosure provides a compound selected from any of the compounds in Table 1, Table 2, Table 3, Table 4 or Table 5, or a pharmaceutically acceptable salt thereof.

TABLE 1
Compounds of Formula (II)

| Compound | Structure |
|---|---|
| 79 | 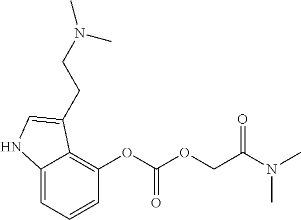 [SMILES: CN(C)CCc1c[nH]c2cccc(OC(=O)OCC(=O)N(C)C)c12] |
| 80 | 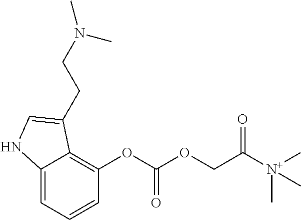 [SMILES: CN(C)CCc1c[nH]c2cccc(OC(=O)OCC(=O)[N+](C)(C)C)c12] |
| 81 | 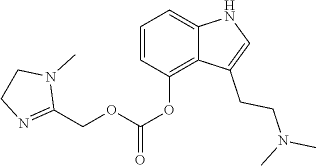 [SMILES: CN(C)CCc1c[nH]c2cccc(OC(=O)OCC3=NCCN3C)c12] |
| 82 | 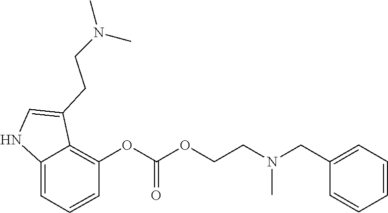 [SMILES: CN(C)CCc1c[nH]c2cccc(OC(=O)OCCN(C)Cc3ccccc3)c12] |
| 83 | 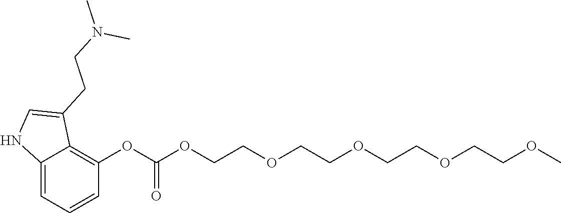 [SMILES: COCCOCCOCCOCCOC(=O)Oc1cccc2[nH]cc(CCN(C)C)c12] |
| 84 | 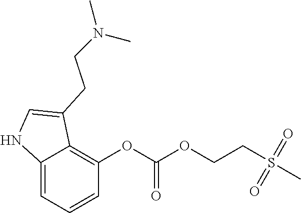 [SMILES: CN(C)CCc1c[nH]c2cccc(OC(=O)OCCS(C)(=O)=O)c12] |
| 85 | 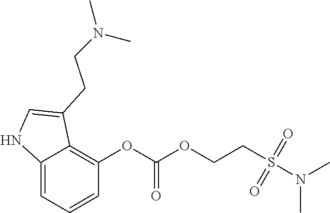 [SMILES: CN(C)CCc1c[nH]c2cccc(OC(=O)OCCS(=O)(=O)N(C)C)c12] |
| 86 | 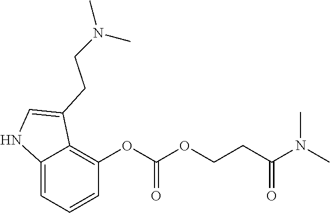 [SMILES: CN(C)CCc1c[nH]c2cccc(OC(=O)OCCC(=O)N(C)C)c12] |
| 87 | 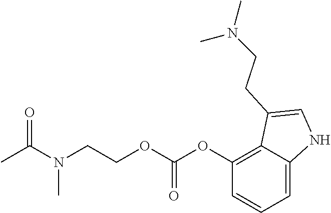 [SMILES: CC(=O)N(C)CCOC(=O)Oc1cccc2[nH]cc(CCN(C)C)c12] |
| 106 | 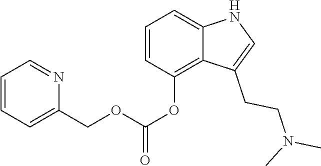 [SMILES: CN(C)CCc1c[nH]c2cccc(OC(=O)OCc3ccccn3)c12] |
| 107 | 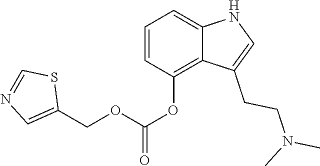 [SMILES: CN(C)CCc1c[nH]c2cccc(OC(=O)OCc3cncs3)c12] |
| 108 | 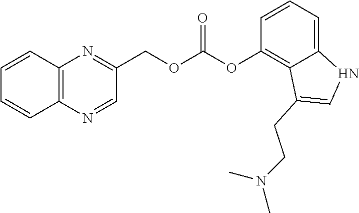 [SMILES: CN(C)CCc1c[nH]c2cccc(OC(=O)OCc3cnc4ccccc4n3)c12] |
| 109 | 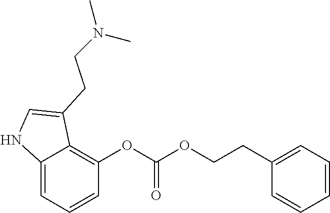 [SMILES: CN(C)CCc1c[nH]c2cccc(OC(=O)OCCc3ccccc3)c12] |
| 110 | 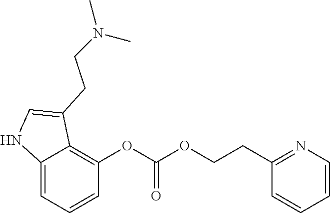 [SMILES: CN(C)CCc1c[nH]c2cccc(OC(=O)OCCc3ccccn3)c12] |
| 111 | 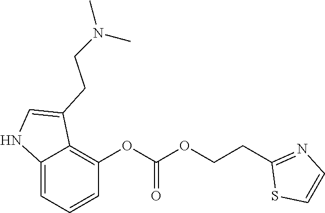 [SMILES: CN(C)CCc1c[nH]c2cccc(OC(=O)OCCc3nccs3)c12] |
| 112 | 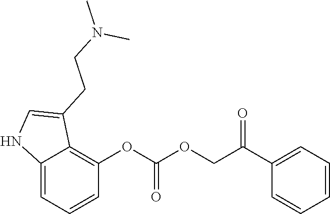 [SMILES: CN(C)CCc1c[nH]c2cccc(OC(=O)OCC(=O)c3ccccc3)c12] |
| 136 | 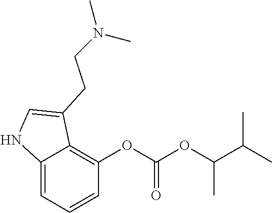 [SMILES: CC(C)C(C)OC(=O)Oc1cccc2[nH]cc(CCN(C)C)c12] |
| 141 | 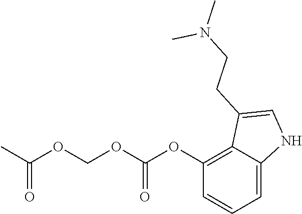 [SMILES: CC(=O)OCOC(=O)Oc1cccc2[nH]cc(CCN(C)C)c12] |
| 142 | 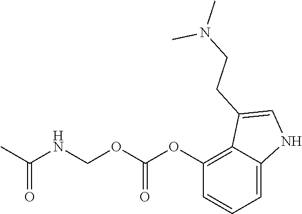 [SMILES: CC(=O)NCOC(=O)Oc1cccc2[nH]cc(CCN(C)C)c12] |
| 144 | 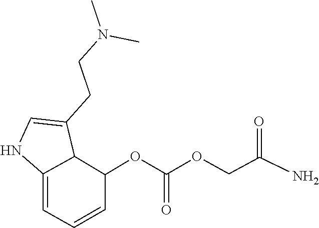 [SMILES: CN(C)CCC1=CNC2=CC=CC(OC(=O)OCC(N)=O)C12] |
| 145 | 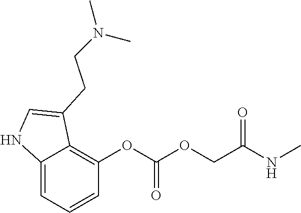 [SMILES: CNC(=O)COC(=O)Oc1cccc2[nH]cc(CCN(C)C)c12] |
| 147 | 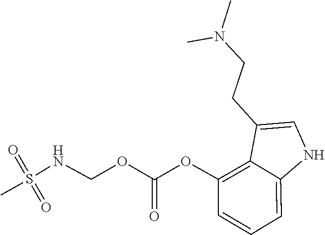 [SMILES: CN(C)CCC1=CNc2cccc(OC(=O)OCNS(C)(=O)=O)c2C1] |
| 148 | 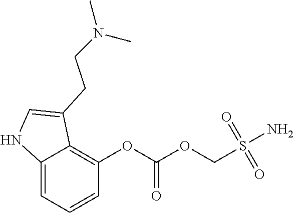 [SMILES: CN(C)CCc1c[nH]c2cccc(OC(=O)OCS(N)(=O)=O)c12] |
| 149 | 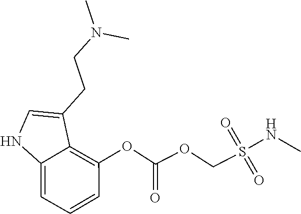 [SMILES: CNS(=O)(=O)COC(=O)Oc1cccc2[nH]cc(CCN(C)C)c12] |
| 150 | 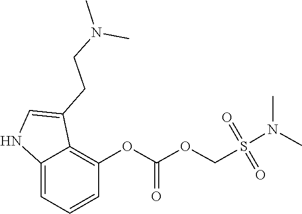 [SMILES: CN(C)CCc1c[nH]c2cccc(OC(=O)OCS(=O)(=O)N(C)C)c12] |
| 151 | 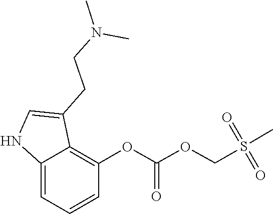 [SMILES: CN(C)CCc1c[nH]c2cccc(OC(=O)OCS(C)(=O)=O)c12] |
| 189 | 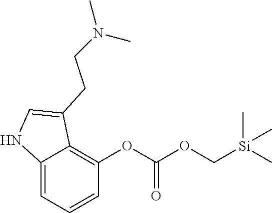 [SMILES: CN(C)CCc1c[nH]c2cccc(OC(=O)OC[Si](C)(C)C)c12] |
| 190 | 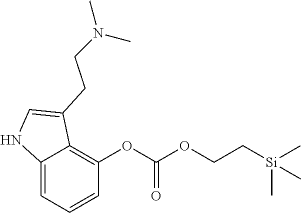 [SMILES: CN(C)CCc1c[nH]c2cccc(OC(=O)OCC[Si](C)(C)C)c12] |
| 191 | 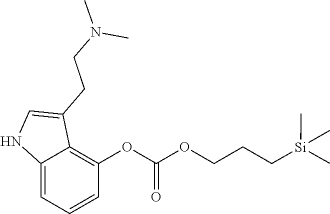 [SMILES: CN(C)CCc1c[nH]c2cccc(OC(=O)OCCC[Si](C)(C)C)c12] |
| 206 | 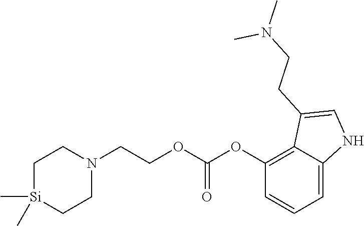 [SMILES: CN(C)CCc1c[nH]c2cccc(OC(=O)OCCN3CC[Si](C)(C)CC3)c12] |
| 203 | 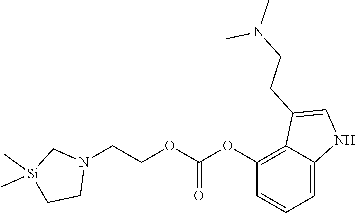 [SMILES: CN(C)CCc1c[nH]c2cccc(OC(=O)OCCN3CC[Si](C)(C)C3)c12] |
| 52 | 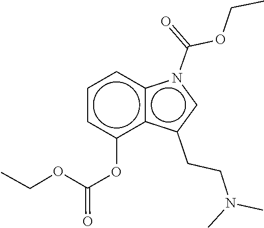 |
| 88 | 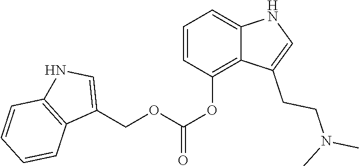 [SMILES: CN(C)CCc1c[nH]c2cccc(OC(=O)OCc3c[nH]c4ccccc34)c12] |
| 89 | 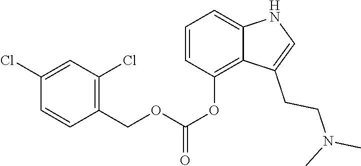 [SMILES: CN(C)CCc1c[nH]c2cccc(OC(=O)OCc3ccc(Cl)cc3Cl)c12] |
| 104 | 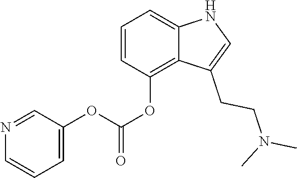 [SMILES: CN(C)CCc1c[nH]c2cccc(OC(=O)Oc3cccnc3)c12] |

TABLE 2
Compounds of Formula (III)

| Compound | Structure |
|---|---|
| 194 | 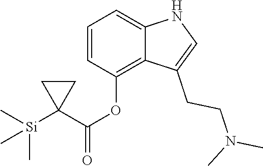 |
| 77 | 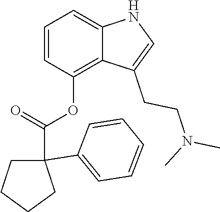 [SMILES: CN(C)CCc1c[nH]c2cccc(OC(=O)C3(c4ccccc4)CCCC3)c12] |
| 90 | 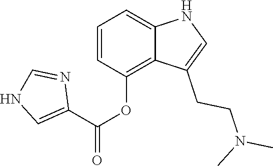 [SMILES: CN(C)CCc1c[nH]c2cccc(OC(=O)c3c[nH]cn3)c12] |
| 91 | 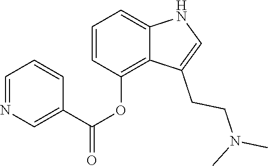 [SMILES: CN(C)CCc1c[nH]c2cccc(OC(=O)c3cccnc3)c12] |
| 92 | 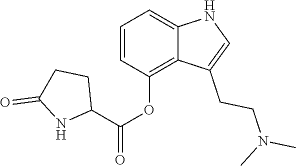 [SMILES: CN(C)CCc1c[nH]c2cccc(OC(=O)C3CCC(=O)N3)c12] |
| 97 | 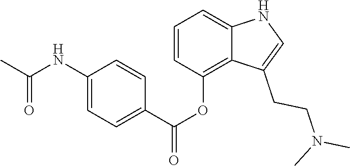 [SMILES: CC(=O)Nc1ccc(C(=O)Oc2cccc3[nH]cc(CCN(C)C)c23)cc1] |
| 99 | 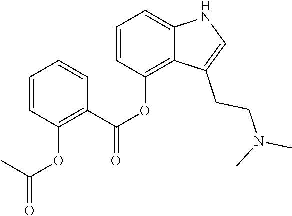 [SMILES: CC(=O)Oc1ccccc1C(=O)Oc1cccc2[nH]cc(CCN(C)C)c12] |
| 113 | 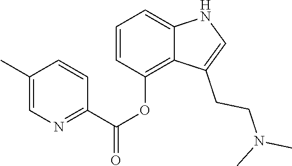 |
| 115 | 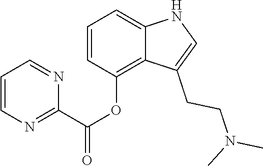 [SMILES: CN(C)CCc1c[nH]c2cccc(OC(=O)c3ncccn3)c12] |
| 116 | 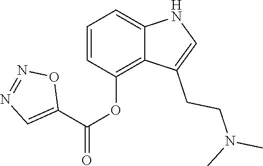 [SMILES: CN(C)CCc1c[nH]c2cccc(OC(=O)c3cnno3)c12] |
| 117 | 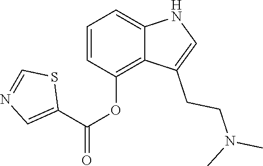 [SMILES: CN(C)CCc1c[nH]c2cccc(OC(=O)c3cncs3)c12] |
| 118 | 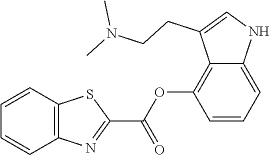 [SMILES: CN(C)CCc1c[nH]c2cccc(OC(=O)c3nc4ccccc4s3)c12] |
| 168 | 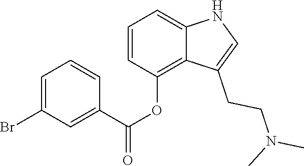 [SMILES: CN(C)CCc1c[nH]c2cccc(OC(=O)c3cccc(Br)c3)c12] |
| 169 | 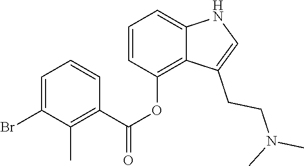 [SMILES: Cc1c(Br)cccc1C(=O)Oc1cccc2[nH]cc(CCN(C)C)c12] |
| 170 | 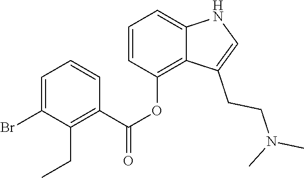 [SMILES: CCc1c(Br)cccc1C(=O)Oc1cccc2[nH]cc(CCN(C)C)c12] |
| 171 | 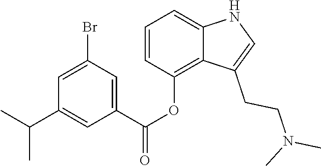 [SMILES: CC(C)c1cc(Br)cc(C(=O)Oc2cccc3[nH]cc(CCN(C)C)c23)c1] |
| 172 | 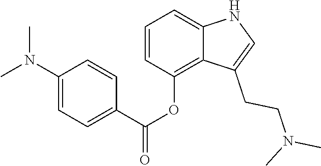 |
| 173 | 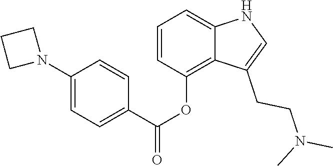 [SMILES: CN(C)CCc1c[nH]c2cccc(OC(=O)c3ccc(N4CCC4)cc3)c12] |
| 174 | 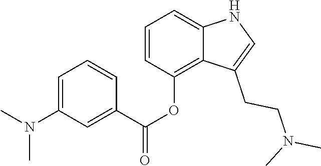 |
| 175 | 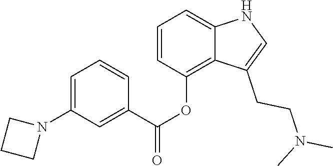 [SMILES: CN(C)CCc1c[nH]c2cccc(OC(=O)c3cccc(N4CCC4)c3)c12] |
| 101 | 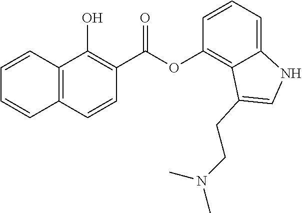 [SMILES: CN(C)CCc1c[nH]c2cccc(OC(=O)c3ccc4ccccc4c3O)c12] |
| 102 | 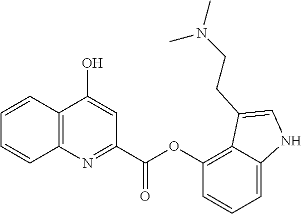 [SMILES: CN(C)CCc1c[nH]c2cccc(OC(=O)c3cc(O)c4ccccc4n3)c12] |
| 188 | 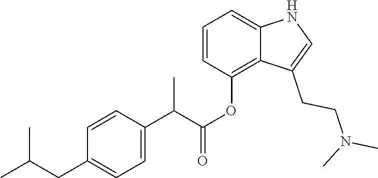 [SMILES: CC(C)Cc1ccc(C(C)C(=O)Oc2cccc3[nH]cc(CCN(C)C)c23)cc1] |
| 76 | 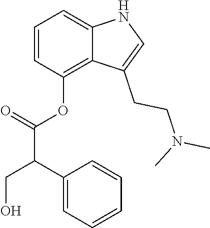 [SMILES: CN(C)CCc1c[nH]c2cccc(OC(=O)C(CO)c3ccccc3)c12] |
| 78 | 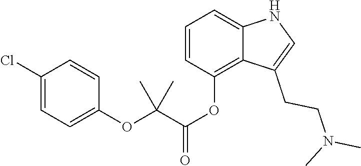 [SMILES: CN(C)CCc1c[nH]c2cccc(OC(=O)C(C)(C)Oc3ccc(Cl)cc3)c12] |
| 93 | 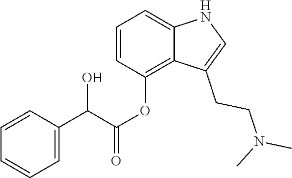 [SMILES: CN(C)CCc1c[nH]c2cccc(OC(=O)C(O)c3ccccc3)c12] |
| 94 | 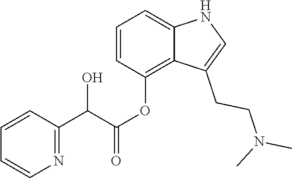 [SMILES: CN(C)CCc1c[nH]c2cccc(OC(=O)C(O)c3ccccn3)c12] |
| 129 | 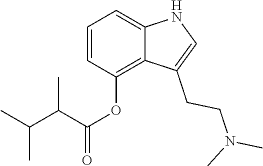 [SMILES: CC(C)C(C)C(=O)Oc1cccc2[nH]cc(CCN(C)C)c12] |
| 182 | 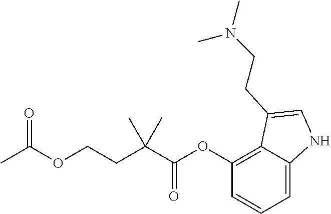 [SMILES: CC(=O)OCCC(C)(C)C(=O)Oc1cccc2[nH]cc(CCN(C)C)c12] |
| 193 | 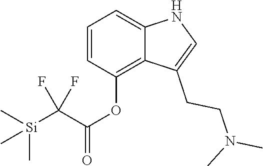 |

TABLE 3
Compounds of Formula (IV)

| Com- | |
|---|---|
| pound | Structure |
| 95 | 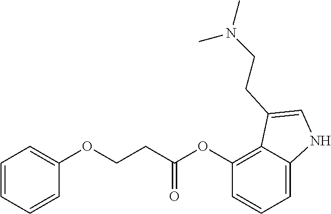 [SMILES: CN(C)CCc1c[nH]c2cccc(OC(=O)CCOc3ccccc3)c12] |
| 96 | 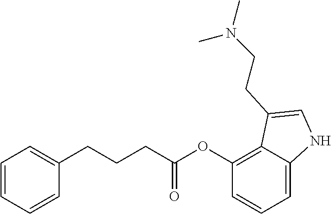 [SMILES: CN(C)CCc1c[nH]c2cccc(OC(=O)CCCc3ccccc3)c12] |
| 98 | 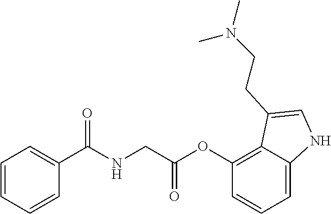 [SMILES: CN(C)CCc1c[nH]c2cccc(OC(=O)CNC(=O)c3ccccc3)c12] |
| 100 | 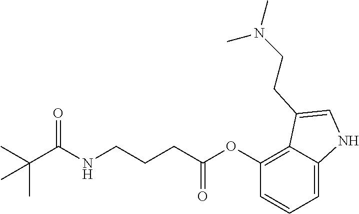 [SMILES: CN(C)CCc1c[nH]c2cccc(OC(=O)CCCNC(=O)C(C)(C)C)c12] |
| 103 | 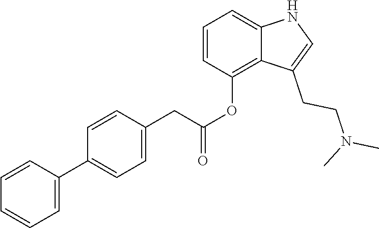 [SMILES: CN(C)CCc1c[nH]c2cccc(OC(=O)Cc3ccc(-c4ccccc4)cc3)c12] |
| 105 | 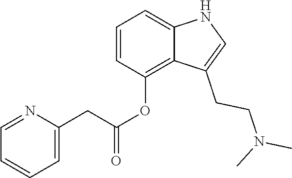 [SMILES: CN(C)CCc1c[nH]c2cccc(OC(=O)Cc3ccccn3)c12] |
| 119 | 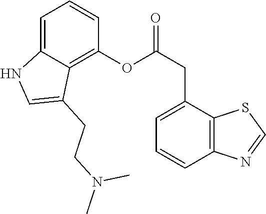 [SMILES: CN(C)CCc1c[nH]c2cccc(OC(=O)Cc3cccc4ncsc34)c12] |
| 120 | 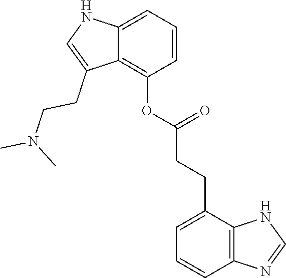 [SMILES: CN(C)CCc1c[nH]c2cccc(OC(=O)CCc3cccc4nc[nH]c34)c12] |
| 121 | 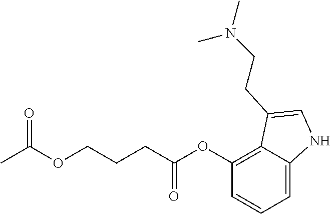 [SMILES: CC(=O)OCCCC(=O)Oc1cccc2[nH]cc(CCN(C)C)c12] |
| 122 | 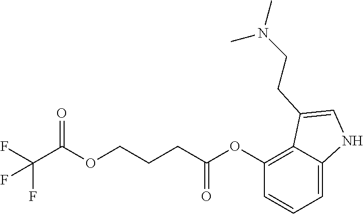 [SMILES: CN(C)CCc1c[nH]c2cccc(OC(=O)CCCOC(=O)C(F)(F)F)c12] |
| 123 | 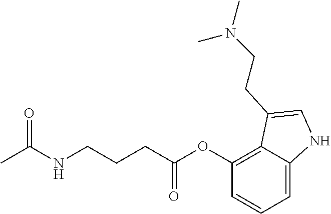 [SMILES: CC(=O)NCCCC(=O)Oc1cccc2[nH]cc(CCN(C)C)c12] |
| 124 | 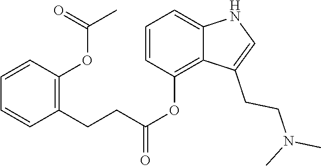 [SMILES: CC(=O)Oc1ccccc1CCC(=O)Oc1cccc2[nH]cc(CCN(C)C)c12] |
| 125 | 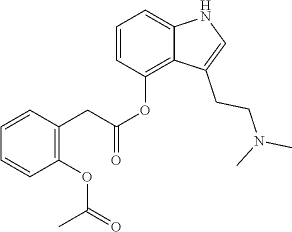 [SMILES: CC(=O)Oc1ccccc1CC(=O)Oc1cccc2[nH]cc(CCN(C)C)c12] |
| 132 | 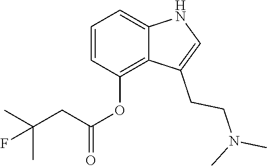 [SMILES: CN(C)CCc1c[nH]c2cccc(OC(=O)CC(C)(C)F)c12] |
| 133 | 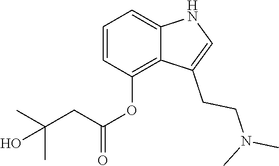 [SMILES: CN(C)CCc1c[nH]c2cccc(OC(=O)CC(C)(C)O)c12] |
| 139 | 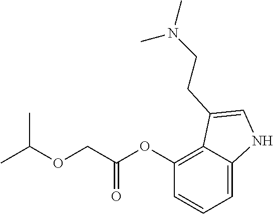 [SMILES: CC(C)OCC(=O)Oc1cccc2[nH]cc(CCN(C)C)c12] |
| 153 | 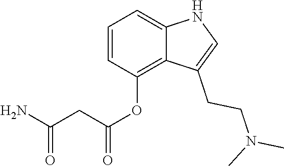 [SMILES: CN(C)CCc1c[nH]c2cccc(OC(=O)CC(N)=O)c12] |
| 154 | 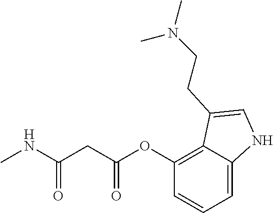 [SMILES: CNC(=O)CC(=O)Oc1cccc2[nH]cc(CCN(C)C)c12] |
| 155 | 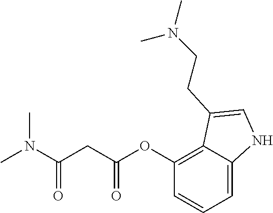 [SMILES: CN(C)CCc1c[nH]c2cccc(OC(=O)CC(=O)N(C)C)c12] |
| 156 | 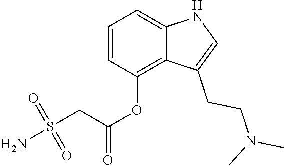 [SMILES: CN(C)CCc1c[nH]c2cccc(OC(=O)CS(N)(=O)=O)c12] |
| 157 | 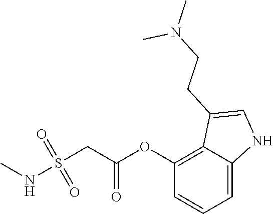 [SMILES: CNS(=O)(=O)CC(=O)Oc1cccc2[nH]cc(CCN(C)C)c12] |
| 158 | 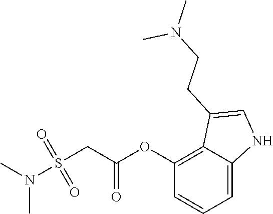 [SMILES: CN(C)CCc1c[nH]c2cccc(OC(=O)CS(=O)(=O)N(C)C)c12] |
| 159 | 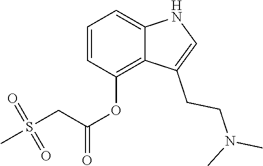 |
| 163 | 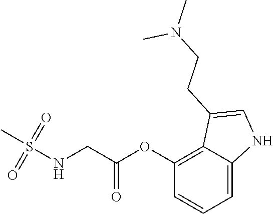 [SMILES: CN(C)CCc1c[nH]c2cccc(OC(=O)CNS(C)(=O)=O)c12] |
| 164 | 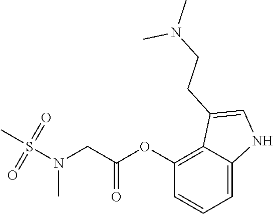 [SMILES: CN(C)CCc1c[nH]c2cccc(OC(=O)CN(C)S(C)(=O)=O)c12] |
| 161 | 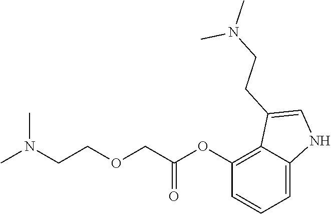 [SMILES: CN(C)CCOCC(=O)Oc1cccc2[nH]cc(CCN(C)C)c12] |
| 162 | 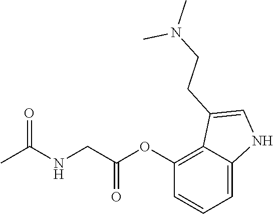 [SMILES: CC(=O)NCC(=O)Oc1cccc2[nH]cc(CCN(C)C)c12] |
| 176 | 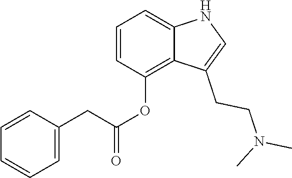 [SMILES: CN(C)CCc1c[nH]c2cccc(OC(=O)Cc3ccccc3)c12] |
| 177 | 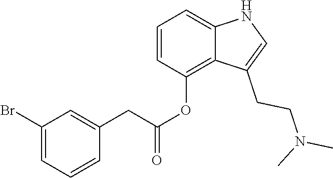 [SMILES: CN(C)CCc1c[nH]c2cccc(OC(=O)Cc3cccc(Br)c3)c12] |
| 178 | 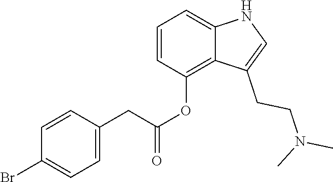 [SMILES: CN(C)CCc1c[nH]c2cccc(OC(=O)Cc3ccc(Br)cc3)c12] |
| 179 | 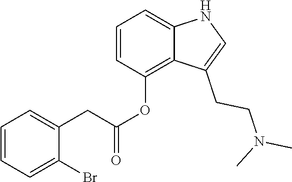 [SMILES: CN(C)CCc1c[nH]c2cccc(OC(=O)Cc3ccccc3Br)c12] |
| 180 | 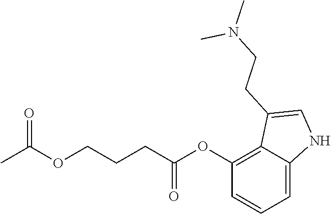 [SMILES: CC(=O)OCCCC(=O)Oc1cccc2[nH]cc(CCN(C)C)c12] |
| 181 | 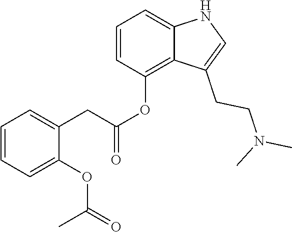 [SMILES: CC(=O)Oc1ccccc1CC(=O)Oc1cccc2[nH]cc(CCN(C)C)c12] |
| 183 | 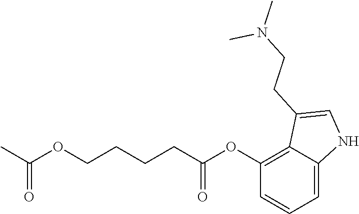 [SMILES: CC(=O)OCCCCC(=O)Oc1cccc2[nH]cc(CCN(C)C)c12] |
| 184 | 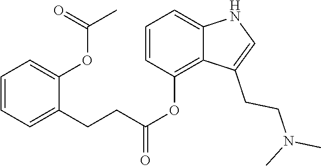 [SMILES: CC(=O)Oc1ccccc1CCC(=O)Oc1cccc2[nH]cc(CCN(C)C)c12] |
| 185 | 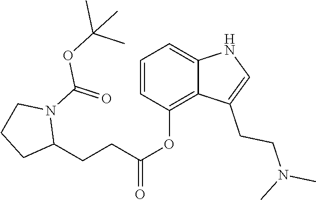 [SMILES: CN(C)CCc1c[nH]c2cccc(OC(=O)CCC3CCCN3C(=O)OC(C)(C)C)c12] |
| 187 | 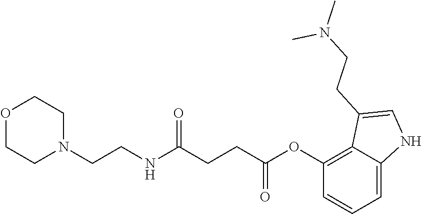 [SMILES: CN(C)CCc1c[nH]c2cccc(OC(=O)CCC(=O)NCCN3CCOCC3)c12] |
| 192 | 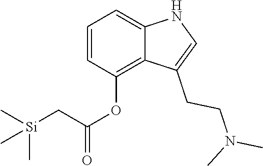 |
| 201 | 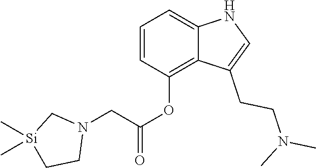 |

TABLE 4
Compounds of Formula (V)

| Compound | Structure |
|---|---|
| 137 | 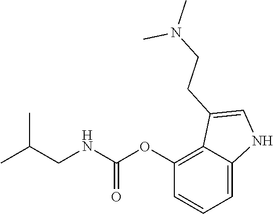 [SMILES: CC(C)CNC(=O)Oc1cccc2[nH]cc(CCN(C)C)c12] |
| 165 | 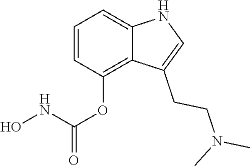 [SMILES: CN(C)CCc1c[nH]c2cccc(OC(=O)NO)c12] |
| 166 | 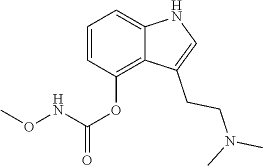 |
| 167 | 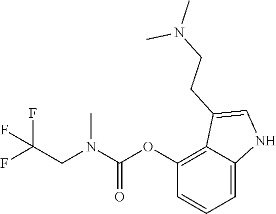 [SMILES: CN(C)CCc1c[nH]c2cccc(OC(=O)N(C)CC(F)(F)F)c12] |
| 135 | 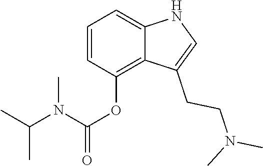 [SMILES: CC(C)N(C)C(=O)Oc1cccc2[nH]cc(CCN(C)C)c12] |
| 138 | 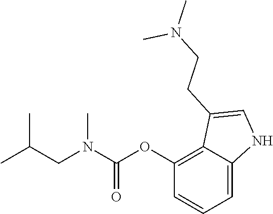 [SMILES: CC(C)CN(C)C(=O)Oc1cccc2[nH]cc(CCN(C)C)c12] |
| 186 | 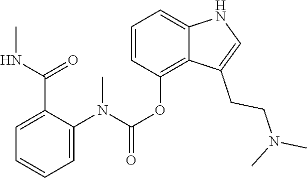 [SMILES: CNC(=O)c1ccccc1N(C)C(=O)Oc1cccc2[nH]cc(CCN(C)C)c12] |
| 202 | 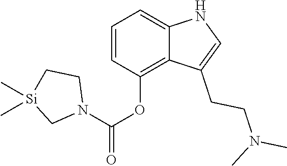 |
| 204 | 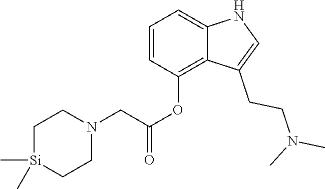 |
| 205 | 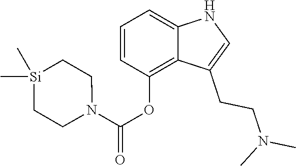 |

TABLE 5
Compounds of Formula (VI)

| Compound | Structure |
|---|---|
| 126 | 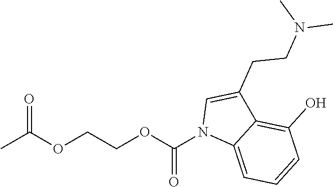 [SMILES: CC(=O)OCCOC(=O)n1cc(CCN(C)C)c2c(O)cccc21] |
| 127 | 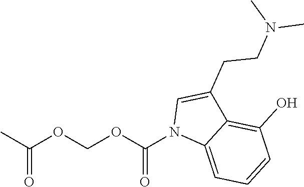 [SMILES: CC(=O)OCOC(=O)n1cc(CCN(C)C)c2c(O)cccc21] |
| 128 | 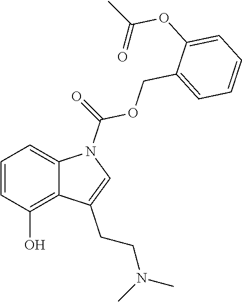 [SMILES: CC(=O)Oc1ccccc1COC(=O)n1cc(CCN(C)C)c2c(O)cccc21] |
| 195 | 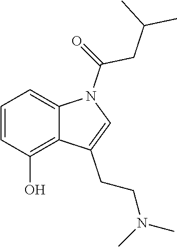 [SMILES: CC(C)CC(=O)n1cc(CCN(C)C)c2c(O)cccc21] |
| 196 | 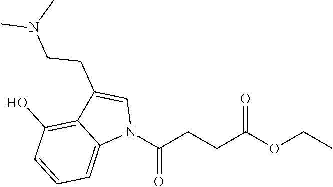 [SMILES: CCOC(=O)CCC(=O)n1cc(CCN(C)C)c2c(O)cccc21] |
| 197 | 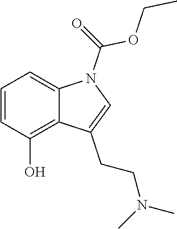 [SMILES: CCOC(=O)n1cc(CCN(C)C)c2c(O)cccc21] |
| 198 | 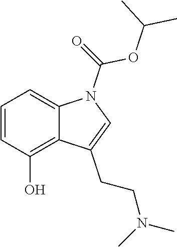 [SMILES: CC(C)OC(=O)n1cc(CCN(C)C)c2c(O)cccc21] |
| 199 | 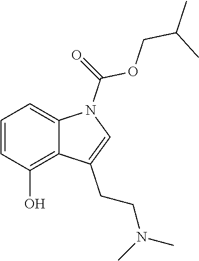 [SMILES: CC(C)COC(=O)n1cc(CCN(C)C)c2c(O)cccc21] |
| 200 | 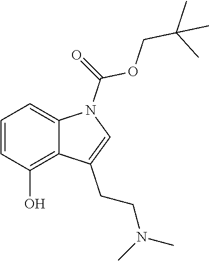 [SMILES: CN(C)CCc1cn(C(=O)OCC(C)(C)C)c2cccc(O)c12] |

### Pharmaceutical Composition

The present disclosure provides pharmaceutical compositions comprising at least one compound disclosed herein and one or more pharmaceutically acceptable excipients.

The compounds provided herein may be administered as compounds per se or may be formulated as pharmaceutical compositions. The pharmaceutical compositions may comprise one or more pharmaceutically acceptable excipients, such as carriers, diluents, fillers, disintegrants, lubricating agents, binders, colorants, pigments, stabilizers, preservatives, and/or antioxidants.

The pharmaceutical compositions can be formulated by techniques known to the person skilled in the art, such as the techniques published in “Remington: The Science and Practice of Pharmacy”, Pharmaceutical Press, 22^{nd} edition. The pharmaceutical compositions can be formulated as dosage forms for oral, parenteral, such as intramuscular, intravenous, subcutaneous, intradermal, intraarterial, intracardial, rectal, nasal, topical, aerosol or vaginal administration. Dosage forms for oral administration include coated and uncoated tablets, soft gelatin capsules, hard gelatin capsules, lozenges, troches, solutions, emulsions, suspensions, syrups, elixirs, powders and granules for reconstitution, dispersible powders and granules, medicated gums, chewing tablets and effervescent tablets. Dosage forms for parenteral administration include solutions, emulsions, suspensions, dispersions and powders and granules for reconstitution. Emulsions are a preferred dosage form for parenteral administration. Dosage forms for rectal and vaginal administration include suppositories and ovula. Dosage forms for nasal administration can be administered via inhalation and insufflation, for example by a metered inhaler. Dosage forms for topical administration include creams, gels, ointments, salves, patches and transdermal delivery systems.

### Method of Treatment

The disclosure further relates to compounds disclosed herein, or a pharmaceutical composition comprising at least one compound disclosed here, for use in the treatment of a serotonin 5-HT2A receptor associated disease/disorder. In embodiments, the compounds may be used in the treatment of an anxiety disorder, attention deficit hyperactivity disorder (ADHD), depression (including treatment resistant depression), cluster headache, diminished drive, burn-out, bore-out, migraine, Parkinson's disease, schizophrenia, an eating disorder (including anorexia nervosa), psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, social anxiety disorder, substance-induced anxiety disorder, selective mutism, panic disorder, panic attacks, agoraphobia, posttraumatic stress disorder (PTSD), premenstrual dysphoric disorder (PMDD), and premenstrual syndrome (PMS).

In embodiments, the 5-HT₂A receptor associated disease or disorder is depression. In embodiments, the depression is treatment resistant depression.

In embodiments, the 5-HT₂A receptor associated disease or disorder is an eating disorder. In embodiments, the eating disorder is anorexia nervosa.

In embodiments, the 5-HT₂A receptor associated disease or disorder is an anxiety disorder.

In embodiments, the 5-HT₂A receptor associated disease or disorder is bipolar I disorder.

In embodiments, the 5-HT₂A receptor associated disease or disorder is bipolar II disorder.

In embodiments, the 5-HT₂A receptor associated disease or disorder is major depressive disorder.

In embodiments, the 5-HT₂A receptor associated disease or disorder is posttraumatic stress disorder (PTSD).

### EXAMPLES

### Example 1. Synthesis of compound 100: 3-(2-(dimethylamino)ethyl)-1H-indol-4-yl 4-pivalamidobutanoate, Formic Acid

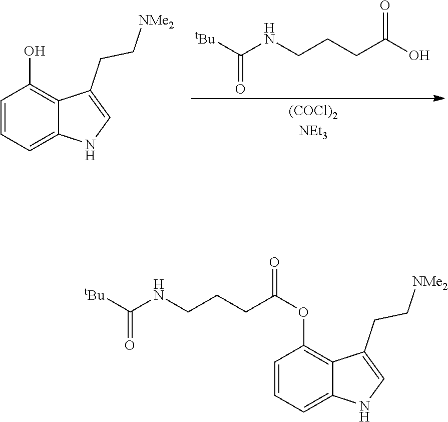 [SMILES: CN(C)CCc1c[nH]c2cccc(O)c12 ; CC(C)(C)C(=O)NCCCC(=O)O ; CN(C)CCc1c[nH]c2cccc(OC(=O)CCCNC(=O)C(C)(C)C)c12]

To a stirred solution of 4-[(2,2-Dimethylpropionyl)amino]butyric acid (187 mg, 1.1 Eq, 999 μmol) in dry DCM (4 mL) under a nitrogen atmosphere at r.t. was added oxalyl chloride (173 mg, 120 μL, 1.5 Eq, 1.37 mmol) and a drop of DMF. The reaction mixture was stirred at r.t. for 3 h. The volatiles were removed in vacuo. The residue was redissolved in DCM (2 mL) and added to a solution of psilocin (204.6 mg, 1 Eq, 942 μmol) and triethylamine (363 mg, 500 μL, 3.8 Eq, 3.59 mmol) in DCM (2 mL) at 0° C. The resulting mixture was stirred at r.t. for 2 h. The reaction mixture was diluted with water (10 mL), extracted with DCM (3×10 mL). The combined organic layers were collected, dried (Na₂SO₄), filtered and concentrated in vacuo. The crude product was purified by chromatography on silica gel (24 g cartridge, 0-20% MeOH/DCM) to afford partially purified product. The material was dissolved in DMSO (1.94 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mLmin⁻¹ eluting with a 0.1% formic acid in water-MeCN gradient over 8.5 min using UV across all wavelengths with PDA as well as a QDA and ELS detector. At column dilution pump gives 2 mLmin⁻¹ MeCN over the entire method, which is included in the following MeCN percentages. Gradient information: 0.0-0.5 min, 10% MeCN; 0.5-5.5 min, ramped from 10% MeCN to 40% MeCN; 5.5-5.6 min, ramped from 40% MeCN to 100% MeCN; 5.6-8.5 min, held at 100% MeCN. The clean fractions were evaporated in a Genevac to afford the title compound (25.7 mg, 52 μmol, 6%) was afforded as a yellow oil.

### m/z 374.3 (M+H)⁺(ES+)

¹H NMR (500 MHz, DMSO-d₆) δ 11.02 (s, 1H), 8.17 (s, 1H), 7.55 (t, J=5.7 Hz, 1H), 7.22 (dd, J=8.1, 0.8 Hz, 1H), 7.14 (d, J=2.3 Hz, 1H), 7.03 (t, J=7.9 Hz, 1H), 6.67 (dd, J=7.6, 0.8 Hz, 1H), 3.16 (q, J=6.5 Hz, 2H), 2.79-2.71 (m, 2H), 2.64 (t, J=7.6 Hz, 2H), 2.23 (s, 6H), 1.85-1.77 (m, 2H), 1.10 (s, 9H). 2H not observed—signal overlaps with DMSO solvent peak. 1× exchangeable H not observed.

### Example 2. Synthesis of compound 105: 3-(2-(dimethylamino)ethyl)-1H-indol-4-yl 2-(pyridin-2-yl)acetate, Formic Acid

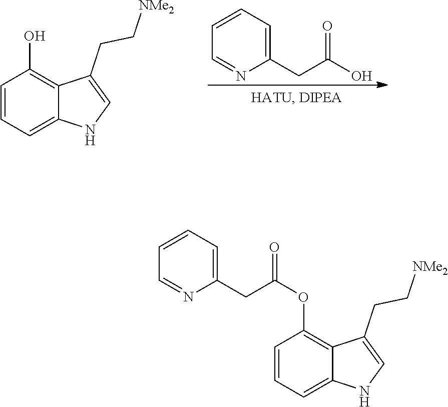 [SMILES: CN(C)CCc1c[nH]c2cccc(O)c12 ; O=C(O)Cc1ccccn1 ; CN(C)CCc1c[nH]c2cccc(OC(=O)Cc3ccccn3)c12]

A mixture of psilocin (200.0 mg, 1 Eq, 960 μmol), 2-(pyridin-2-yl)acetic acid, HCl (168 mg, 1 Eq, 960 μmol), HATU (525 mg, 1.4 Eq, 1.38 mmol) and DIPEA (417 mg, 557 μL, 3.4 Eq, 3.22 mmol) in dry DMF (2.5 mL) was stirred at r.t. for 16 h. The reaction mixture was diluted with sat. NaHCO: (25 mL) and extracted with EtOAc (3×25 mL). The combined organic layers were washed with water (3×20 mL) and brine (25 mL), dried (Na₂SO₄), filtered, and concentrated in vacuo. The crude product was loaded onto celite and purified by chromatography on RP Flash C18 (12 g cartridge, 0-100% (0.1% formic acid in MeCN)/(0.1% formic acid in water)). The partially purified sample was dissolved in DMSO (1.8 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mLmin⁻¹ eluting with a 0.1% formic acid in water-MeCN gradient over 12.5 min using UV across all wavelengths with PDA as well as a QDA and ELS detector. At column dilution pump gives 2 mLmin⁻¹ MeCN over the entire method, which is included in the following MeCN percentages. Gradient information: 0.0-0.5 min, 5% MeCN; 0.5-10.5 min, ramped from 5% MCCN to 22.5% MeCN; 10.5-10.6 min, ramped from 22.5% MeCN to 100% MeCN; 10.6-12.5 min, held at 100% MeCN. The clean fractions were evaporated in a Genevac to afford the title compound (7.0 mg, 17 μmol, 2%) as a brown solid.

m/z 324.1 (M+H)⁺ (ES+)

¹H NMR (500 MHz, DMSO-d₆) δ 11.08 (s, 1H), 8.56 (dd, J=5.1, 1.8 Hz, 1H), 8.24 (s, 1H), 7.85-7.78 (m, 1H), 7.50 (d, J=7.8 Hz, 1H), 7.33 (dd, J=7.6, 4.9 Hz, JH), 7.24 (d, J=8.1 Hz, 1H), 7.17 (d, J=2.3 Hz, 1H), 7.07-7.01 (m, 1H), 6.69 (d, J=7.6 Hz, 1H), 4.23 (s, 2H), 2.87-2.81 (m, 2H), 2.68-2.62 (m, 2H), 2.35 (s, 6H). (1× exchangeable H not observed).

### Example 3. Synthesis of compound 107: 3-(2-(dimethylamino)ethyl)-1H-indol-4-yl (thiazol-5-ylmethyl) carbonate, 0.5Fumaric acid

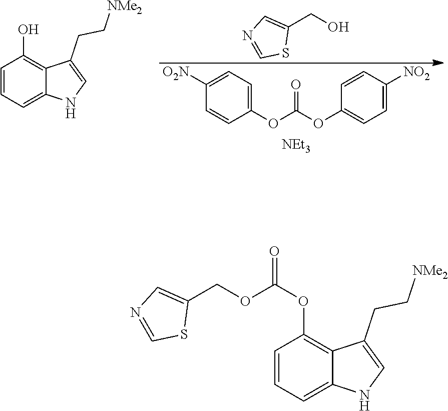

To a stirred solution of thiazol-5-ylmethanol (114 mg, 1 Eq, 990 μmol) and bis(4-nitrophenyl) carbonate (361 mg, 1.2 Eq, 1.19 mmol) in dry DCM (8 mL) under a nitrogen atmosphere at 20° C. was added triethylamine (220 mg, 304 μL, 2.2 Eq, 2.18 mmol). The reaction mixture was stirred for 1 h at r.t. To the reaction mixture under a nitrogen atmosphere at 20° C. was added a solution of psilocin (202.2 mg, 1 Eq, 990 μmol) in DMF (2 mL). The reaction mixture was stirred for 1 h at rt. The reaction mixture was then heated at 38° C. overnight. The reaction mixture was diluted with DCM (10 mL), poured into ice/water (20 mL) and transferred into a separating funnel. The aqueous layer was extracted with DCM (3×10 mL). The combined organic layers were washed with brine (15 mL), dried (Na₂SO₄), filtered and concentrated in vacuo. The crude product was purified by chromatography on RP Flash C18 (24 g cartridge, 5-50% (0.1% formic acid in MeCN)/(0.1% formic acid in water)) to afford 3-(2-(dimethylamino)ethyl)-1H-indol-4-yl (thiazol-5-ylmethyl) carbonate, Formic Acid (94.7 mg, 242 μmol, 24%) as a light yellow oil. The material was dissolved in acetone (5 mL) and a solution of fumaric acid (28 mg, 0.24 Eq, 242 μmol) in acetone (5 mL) was added. The resulting solid was filtered, washed with acetone and dried in vacuo for 24 h to afford the title compound (48.7 mg, 0.12 mmol, 12%) as an off-white solid.

m/z 346.1 (M+H)⁺ (ES+)

¹H NMR (500 MHz, DMSO-d₆) δ 11.13 (s, 1H), 9.18 (s, 1H), 8.05 (s, 1H), 7.30-7.25 (m, 1H), 7.19 (d, J=2.3 Hz, 1H), 7.09-7.02 (m, 1H), 6.79 (d, J=7.6 Hz, 1H), 6.54 (s, 1H), 5.56 (s, 2H), 2.75-2.69 (m, 2H), 2.56-2.51 (m, 2H), 2.20 (s, 6H). 1× exchangeable H not observed.

### Example 4. Synthesis of compound 109: 3-(2-(Dimethylamino)ethyl)-1H-indol-4-yl phenethyl carbonate, Fumaric acid

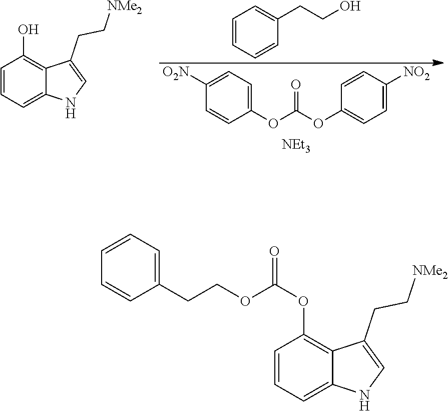

To a stirred solution of 2-phenylethan-1-ol (121.8 mg, 1 Eq, 996.7 μmol) and bis(4-nitrophenyl) carbonate (363.9 mg, 1.2 Eq, 1.196 mmol) in dry DCM (8.00 mL) under a nitrogen atmosphere at 20° C. was added Et₃N (221.9 mg, 306 μL, 2.2 Eq, 2.193 mmol). The reaction mixture was stirred for 1 h at rt. To the reaction mixture, under a nitrogen atmosphere at 20° C., was added a solution of psilocin (203.6 mg, 1 Eq, 996.7 μmol) in DMF (2.00 mL). The reaction mixture was stirred for 1 h at rt. The reaction mixture was then heated at 38° C. for 17 h. The reaction mixture was cooled to rt, diluted with DCM (10 mL), poured into ice/water (30 mL) and transferred into a separating funnel. The aqueous layer was extracted with DCM (3×15 mL). The combined organic layers were washed with water (3×20 mL) and brine (20 mL), dried (Na₂SO₄), filtered and concentrated in vacuo. The crude product was purified by chromatography on RP Flash C18 (12 g cartridge, 5-35% (0.1% formic acid in MeCN)/(0.1% formic acid in water)) to afford a formic acid salt (173.1 mg, 0.35 mmol, 35%, 80% Purity) as a brown oil. The material was dissolved in acetone (8 mL) and a solution of fumaric acid (50.42 mg, 0.4358 Eq, 434.4 μmol) in acetone (6 mL) was added. The resulting solid was filtered, washed with acetone and dried in a vacuum desiccator over weekend to afford the title compound (111.5 mg, 0.23 mmol, 23%, 95% Purity) as a brown solid.

m/z 353.15 (M+H)⁺ (ES+); 307.80 (M-NMe₂—H)⁻ (ES−)

¹H NMR (500 MHz, DMSO) δ 11.17 (d, J=2.7 Hz, 1H), 7.36-7.22 (m, 6H), 7.20 (d, J=2.4 Hz, 1H), 7.05 (t, J=7.9 Hz, 1H), 6.75 (dd, J=7.7, 0.9 Hz, 1H), 6.54 (d, J=1.4 Hz, 2H), 4.46 (t, J=6.8 Hz, 2H), 3.01 (t, J=6.8 Hz, 2H), 2.84 (dd, J=10.0, 5.6 Hz, 2H), 2.74 (dd, J=9.2, 6.1 Hz, 2H), 2.38 (s, 6H). 2H are absent (exchangeable protons of fumaric acid).

### Example 5. Synthesis of compound 110: 3-(2-(dimethylamino)ethyl)-1H-indol-4-yl (2-(pyridin-2-yl)ethyl) carbonate, 2Formic Acid

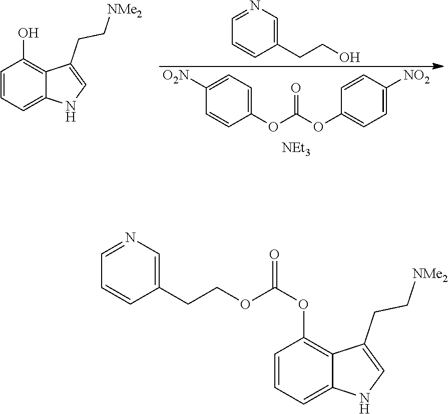

To a solution of 2-(pyridin-2-yl)ethan-1-ol (116 mg, 1 Eq, 920 μmol) and bis(4-nitrophenyl) carbonate (297 mg, 1.1 Eq, 966 μmol) in dry DMF (2 mL) under a nitrogen atmosphere at r.t. was added DIPEA (742 mg, 1.0 mL, 6.2 Eq, 5.74 mmol). The reaction mixture was stirred at r.t. for 2 h. To the reaction mixture was added a solution of psilocin (200 mg, 1 Eq, 920 μmol) in DMF (2 mL). The reaction mixture was stirred at 40° C. for 48 h. The reaction mixture was diluted with distilled water (25 mL). The layer was extracted with DCM (3×20 mL). The combined organic layers were collected, dried (Na₂SO₄), filtered and concentrated in vacuo. The crude product was loaded onto celite and purified by chromatography on RP Flash C18 (12 g cartridge, 5-20% (0.1% formic acid in MeCNY(0.1% formic acid in water)) to afford the title compound (196 mg, 0.38 mmol, 42%) as a dark brown oil.

m/z 354.2 (M+H)⁺ (ES+)

¹H NMR (500 MHz, DMSO-d₆) δ 11.13 (s, 1H), 8.53 (ddd, J=4.9, 1.9, 0.9 Hz, 1H), 8.21 (s, 2H), 7.75 (td, J=7.6, 1.9 Hz, 1H), 7.35 (dt, J=7.8, 1.1 Hz, 1H), 7.29-7.24 (m, 2H), 7.19 (d, J=2.3 Hz, 1H), 7.07-7.01 (m, 1H), 6.74 (dd, J=7.7, 0.8 Hz, 1H), 4.63 (t, J=6.6 Hz, 2H), 3.17 (d, J=13.1 Hz, 2H), 2.78 (dd, J=9.2, 6.5 Hz, 2H), 2.63 (dd, J=9.2, 6.5 Hz, 2H), 2.30 (s, 6H). 2× exchangeable H not observed.

### Example 6. Synthesis of compound 113: 3-(2-(dimethylamino)ethyl)-1H-indol-4-yl 5-methylpicolinate, 0.5Fumaric acid

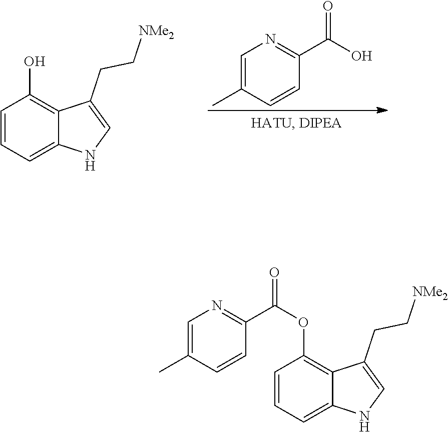

A mixture of psilocin (202.0 mg, 1 Eq, 930 μmol), 5-methylpicolinic acid (130 mg, 1 Eq, 930 μmol), HATU (509 mg, 1.4 Eq, 1.34 mmol) and DIPEA (284 mg, 379 μL, 2.4 Eq, 2.19 mmol) in dry DMF (2.5 mL) was stirred at r.t. for 7 days. The reaction mixture was diluted with sat. NaHCO₃(25 mL), and extracted with EtOAc (3×25 mL). The combined organic layers were washed with water (3×20 mL) and brine (25 mL), dried (Na₂SO₄), filtered, and concentrated in vacuo. The crude product was loaded onto celite and purified by chromatography on RP Flash C18 (12 g cartridge, 5-30% (0.1% formic acid in MeCN)/(0.1% formic acid in water)) to afford 3-(2-(dimethylamino)ethyl)-1H-indol-4-yl 5-methylpicolinate, formic acid salt (42 mg) a glassy oil. To a solution the material (40.0 mg, 1 Eq, 103 μmol) in acetone (2 mL) was added a solution of fumaric acid (13 mg, 1.05 Eq, 108 μmol) in acetone (2 mL). The stored at −20° C. for 64 h. The resulting solid was isolated by filtration and dried in vacuo at 45° C. for 2 h to afford the title compound (35.2 mg, 90 μmol, 10%) as an off-white solid.

m/z 324.6 (M+H)⁺ (ES+)

¹H NMR (500 MHz, DMSO-d₆) δ 1H NMR (500 MHz, DMSO) δ 11.19-11.05 (m, 1H), 8.70-8.65 (m, 1H), 8.20 (d, J=7.9 Hz, 1H), 7.93-7.88 (m, 1H), 7.29 (dd, 1H), 7.18 (d, J=2.3 Hz, 1H), 7.12-7.08 (m, 1H), 6.81 (dd, 1H), 6.52 (s, 1H), 2.77-2.71 (m, 2H), 2.56-2.52 (m, 2H), 2.45 (s, 3H), 2.04 (s, 6H). (1× exchangeable H not observed).

### Example 7. Synthesis of compound 117: 3-(2-(dimethylamino)ethyl)-1H-indol-4-yl thiazole-5-carboxylate, Formic Acid

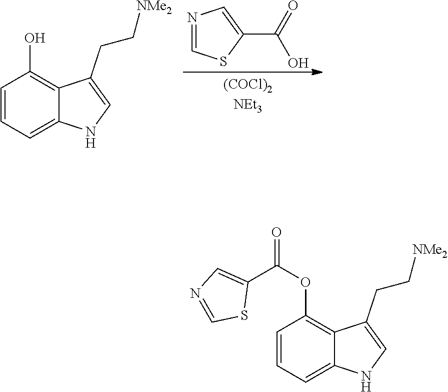 [SMILES: CN(C)CCc1c[nH]c2cccc(O)c12 ; O=C(O)c1cncs1 ; CN(C)CCc1c[nH]c2cccc(OC(=O)c3cncs3)c12]

To a stirred solution of thiazole-5-carboxylic acid (135 mg, 1.1 Eq, 1.04 mmol) in dry DCM (4 mL) under a nitrogen atmosphere at r.t. was added oxalyl chloride (173 mg, 120 μL, 1.5 Eq, 1.37 mmol) and a drop of DMF. The reaction mixture was stirred at r.t. for 3 h. The resulting mixture was stirred at r.t. for 3 h. The volatiles were removed in vacuo. The residue was redissolved in DCM (2 mL) and added to a solution of psilocin (200.1 mg, 1 Eq, 921 μmol) and triethylamine (363 mg, 500 μL, 3.9 Eq, 3.59 mmol) in DCM (2 mL) at 0° C. The resulting mixture was stirred at r.t. for 2 h. The reaction mixture was diluted with water (10 mL), extracted with DCM (3×10 mL). The combined organic layers were collected, dried (Na₂SO₄), filtered and concentrated in vacuo. The crude product was purified by chromatography on RP Flash C18 (24 g cartridge, 5-40% (0.1% formic acid in MeCN)/(0.1% formic acid in water)) to afford partially purified title compound (224.1 mg) as a brown oil. 101.9 mg of the material was triturated with diethyl ether and dried in a vacuum desiccator at 40° C. for 72 h to afford the title compound (66.4 mg, 0.18 mmol, 20%) as a light brown solid.

m/z 316.1 (M+H)⁺ (ES+)

¹H NMR (500 MHz, DMSO-d₆) δ 11.15 (s, 1H), 9.51 (d, J=0.8 Hz, 1H), 8.83 (d, J=0.8 Hz, 1H), 8.17 (s, 1H), 7.31 (dd, J=8.2, 0.8 Hz, 1H), 7.20 (d, J=2.3 Hz, 1H), 7.10 (t, J=7.9 Hz, 1H), 6.85 (dd, J=7.7, 0.8 Hz, 1H), 2.79-2.72 (m, 2H), 2.07 (s, 6H). Two protons from CH₂ not observed. Signal overlaps with DMSO solvent peak. 1× exchangeable H not observed.

### Example 8. Synthesis of compound 118: 3-(2-(Dimethylamino)ethyl)-1H-indol-4-yl benzo[d]thiazole-2-carboxylate, Fumaric acid

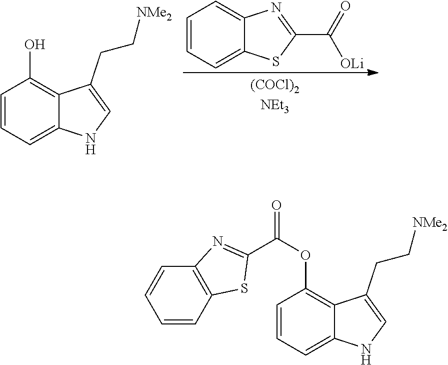

To a stirred solution of lithium benzo[d]thiazole-2-carboxylate (184.2 mg, 1 Eq, 994.8 μmol) in dry DCM (4.00 mL) under an atmosphere of N₂ at rt was added oxalyl chloride (265.1 mg, 182.9 IL, 2.1 Eq, 2.089 mmol) and a drop of DMF (72.72 mg, 77.0 μL, 1 Eq, 994.8 μmol). The reaction mixture was stirred at rt for 2 h. The volatiles were removed in vacuo. The residue was dissolved in DCM (2.00 mL) and added to a solution of psilocin (203.2 mg, 1 Eq, 994.8 μmol) and Et₃N (362.4 mg, 499 μL, 3.6 Eq, 3.581 mmol) in DCM (2.00 mL) at 0° C. The reaction mixture was stirred at room temp for 1 h. The reaction mixture was concentrated in vacuo. The crude product was purified by chromatography on RP Flash C18 (24 g cartridge, 5-40% (0.1% formic acid in MeCN)/(0.1% formic acid in water)) to afford 3-(2-(dimethylamino)ethyl)-1H-indol-4-yl benzo[d]thiazole-2-carboxylate, Formic Acid (199.4 mg, 0.35 mmol, 35%, 72% Purity) as a dark yellow oil. The material was dissolved in acetone (8 mL) and a solution of fumaric acid (46 mg, 0.40 Eq, 0.40 mmol) in acetone (6 mL) was added. The resulting solid was filtered, washed with acetone and dried in a vacuum desiccator overnight to afford the title compound (92.2 mg, 0.17 mmol, 17%, 88% Purity) as a light brown solid.

m/z 366.14 (M+H)⁺ (ES+); 319.88 (M-NMe₂-H)⁻ (ES−)

¹H NMR (500 MHz, DMSO) δ 11.24 (s, 1H), 8.37-8.29 (m, 2H), 7.77-7.67 (m, 2H), 7.36 (d, J=8.1 Hz, 1H), 7.25 (d, J=2.3 Hz, 1H), 7.18-7 11 (m, 1H), 6.99 (d, J=7.6 Hz, 1H), 6.56 (s, 2H), 2.90-2.84 (m, 2H), 2.73 (s, 2H), 2.17 (s, 6H). 2H not observed (exchangeable protons of fumaric acid).

### Example 9. Synthesis of compound 123: 3-(2-(dimethylamino)ethyl)-1H-indol-4-yl 4-acetamidobutanoate, 0.2 HPF6, 0.8 Formic acid

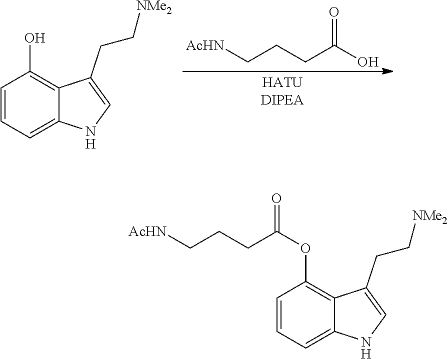 [SMILES: CN(C)CCc1c[nH]c2cccc(O)c12 ; CC(=O)NCCCC(=O)O ; CC(=O)NCCCC(=O)Oc1cccc2[nH]cc(CCN(C)C)c12]

A mixture of 4-acetamidobutanoic acid (141 mg, 1 Eq, 969 μmol), HATU (516 mg, 1.4 Eq, 1.36 mmol), DIPEA (301 mg, 405 μL, 2.4 Eq, 2.33 mmol) and psilocin (200 mg, 1 Eq, 969 μmol) in DMF (4 mL) was stirred at r.t overnight. The reaction mixture was directly loaded on to a 80 g C18 reverse column and purified (80 g cartridge, 0-100% (0.1% formic acid in MeCN)/(0.1% formic acid in water)) to afford the desired product as an amber glass with a DMF impurity. Upon standing the desired product crystallized out of the amber glass. The amber glass/crystalline product was triturated in diethyl ether to afford the title compound (25.0 mg, 63.1 μmol, 7%) as a beige solid.

m/z 332.2 (M+H)⁺ (ES+)

¹H NMR (500 MHz, DMSO-d₆) δ 11.23 (d, J=2.5 Hz, 1H), 9.26 (s, 1H), 7.96 (s, 1H), 7.28-7.26 (m, 2H), 7.08 (dd, J=7.9 Hz, 1H), 6.75 (d, J=7.6 Hz, 1H), 3.16 (q, J=6.6 Hz, 2H), 3.02 (t, J=7.9 Hz, 2H), 2.84 (s, 6H), 2.74-2.67 (m, 2H), 1.83 (s, 3H), 1.82-1.78 (m, 2H). 2H obscured by water peak. 1× exchangeable H not observed.

### Example 10. Synthesis of compound 126: 2-Acetoxyethyl 3-(2-(dimethylamino)ethyl)-4-hydroxy-1H-indole-1-carboxylate, Formic Acid

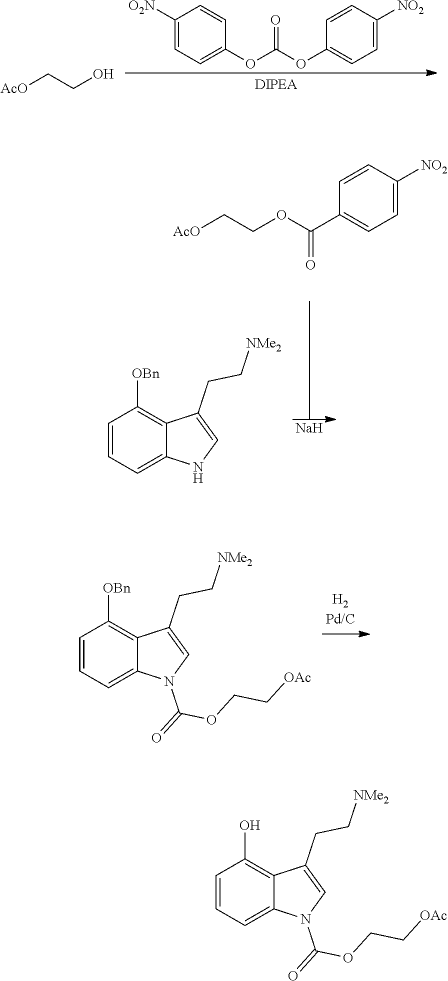

### Step 1: 2-(((4-Nitrophenoxy)carbonyl)oxy)ethyl acetate

To a solution of ethylene glycol monoacetate (100 mg, 90.0 μL, 1 Eq, 961 μmol) and carbonic acid, bis(4-nitrophenyl)ester (315.5 mg, 1.08 Eq, 1.037 mmol) in dry DMF (3.0 mL) at rt was added DIPEA (742 mg, 1.00 mL, 5.98 Eq, 5.74 mmol). The reaction mixture was stirred for 3 h at rt. The reaction mixture was diluted with distilled water (5 mL), extracted with DCM (3×5 mL). The combined organic layers were collected, dried (Na₂SO₄), filtered and concentrated in vacuo. The crude product was purified by chromatography on silica gel (12 g cartridge, 0-50/6 EtOAc/isohexane) to afford the sub-title compound (351.7 mg, 0.61 mmol, 64%, 47% Purity) as a yellow oil.

¹H NMR (500 MHz, DMSO) δ 8.35-8.29 (m, 2H), 7.60-7.53 (m, 2H), 4.48-4.41 (m, 2H), 4.34-4.29 (m, 2H), 2.06 (s, 3H).

### Step 2: 2-Acetoxyethyl-4-(benzyloxy)-3-(2-(dimethylamino)ethyl)-1H-indole-1-carboxylate

To a solution of 2-(4-(benzyloxy)-1H-indol-3-yl)-N,N-dimethylethan-1-amine (145.5 mg, 95% Wt, 1 Eq, 469.5 μmol) in DMF (5.00 mL) at 0° C. was added NaH (21.8 mg, 60% Wt, 1.16 Eq, 545 μmol) and the reaction was stirred for 10 min at 0° C. The product from step 1, above (351.7 mg, 47% Wt, 1.308 Eq, 614.0 μmol) was added at 0° C. then stirred at rt for 3 h. The reaction mixture was diluted with distilled water (5 mL), extracted with DCM (3×5 mL). The combined organic layers were collected, dried (Na₂SO₄), filtered and concentrated in vacuo. The crude product was purified by chromatography on silica gel (24 g cartridge, 0-10% MeOH/DCM) to afford the sub-tide compound (142.6 mg, 0.29 mmol, 62%, 86% Purity) as a light-yellow oil.

m/z 425.2 (M+H)⁺ (ES+)

¹H NMR (500 MHz, DMSO) δ 7.69 (d, J=8.3 Hz, 1H), 7.54-7.49 (m, 2H), 7.43-7.39 (m, 2H), 7.38-7.31 (m, 2H), 7.24 (t, J=8.2 Hz, 1H), 6.90 (d, J=8.0 Hz, 1H), 5.22 (s, 2H), 4.58-4.54 (m, 2H), 4.42-4.37 (m, 2H), 2.92-2.86 (m, 2H), 2.06 (s, 6H), 2.05 (s, 3H). Two protons from CH₂ not observed. Signal overlaps with DMSO solvent peak.

### Step 3: 2-Acetoxyethyl 3-(2-(dimethylamino)ethyl)-4-hydroxy-1H-indole-1-carboxylate, Formic Acid

A solution of the product from step 2, above (142.6 mg, 86% Wt, 1 Eq, 288.9 μmol) in EtOH (20 mL) was hydrogenated in the H-Cube (Pd/C cat cart) at 5 bar at 60° C., 1 mL/min for 30 min (recirculation). The mixture was concentrated in vacuo to afford impure product. The crude material was dissolved in DMSO (1.91 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mL min-1 eluting with a 0.1% formic acid in water-MeCN gradient over 17.5 mins using UV across all wavelengths with PDA as well as a QDA and ELS detector. At-column dilution pump gives 2 mL min-1 MeOH over the entire method, which is included in the following MeCN percentages. Gradient information: 0.0-0.5 min, 5% MeCN; 0.5-15.5 min, ramped from 5% MeCN to 22.5% MeCN; 15.5-15.6 min, ramped from 22.5% MeCN to 100% MeCN; 15.6-17.5 min, held at 100% MeCN. The clean fractions were evaporated in a Genevac, affording the title compound (35.4 mg, 92 μmol, 32%, 99% Purity) as a light brown oil.

m/z 335.2 (M+H)⁺ (ES+)

¹H NMR (500 MHz, DMSO) δ 8.18 (s, 1H), 7.52 (d, J=8.2 Hz, 1H), 7.32 (s, 1H), 7.09 (t, J=8.0 Hz, 1H), 6.60 (dd, J=7.9, 0.8 Hz, 1H), 4.57-4.52 (m, 2H), 4.40-4.36 (m, 2H), 2.94-2.89 (m, 2H), 2.66-2.60 (m, 2H), 2.26 (s, 6H), 2.05 (s, 3H). 2× exchangeable protons not observed.

### Example 11. Synthesis of compound 129: 3-(2-(dimethylamino)ethyl)-1H-indol-4-yl 2,3-dimethylbutanoate, Fumaric acid

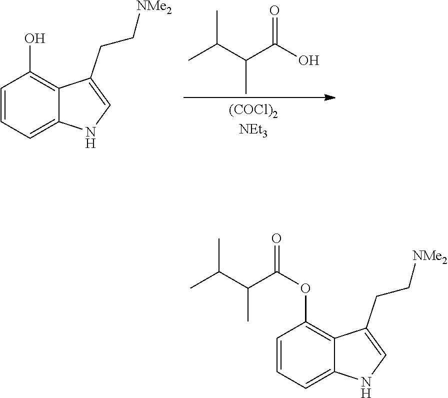

To a stirred solution of 2,3-dimethylbutanoic acid (123 mg, 1.1 Eq, 1.06 mmol) in dry DCM (4 mL) under a nitrogen atmosphere at r.t. was added oxalyl chloride (146 mg, 101 μL, 1.2 Eq, 1.15 mmol) and a drop of DMF. The reaction mixture was stirred at r.t. for 3 h. The volatiles were removed in vacuo. The residue was redissolved in DCM (2 mL) and added to a solution of psilocin (200 mg, 1 Eq, 960 μmol) and triethylamine (485 mg, 669 ILL, 5 Eq, 4.80 mmol) in DCM (2 mL) at 0° C. The resulting mixture was stirred at r.t. for 16 h. The reaction mixture was diluted with water (25 mL) and extracted with DCM (3×25 mL). The combined organic layers were collected, dried (Na₂SO₄), filtered and concentrated in vacuo. The crude product was loaded onto celite and purified by chromatography on RP Flash C18 (24 g cartridge, 0-30% (0.1% formic acid in MeCN)/(0.1% formic acid in water)) to afford 342-(dimethylamino)ethyl)-1H-indol-4-yl 2,3-dimethylbutanoate, formic acid (110 mg, 364 μmol, 38%). The material was dissolved in acetone (6 mL) and a solution of fumaric acid (43 mg, 1.02 Eq, 370 μmol) in acetone (6 mL) was added. The mixture was cooled at −20° C. for 60 h. The resulting solid was isolated by filtration and washed with MeCN (2×2 mL) to afford the title compound (52.3 mg, 0.12 mmol, 13%) as a tan solid.

m/z 303.5 (M+H)⁺ (ES+)

¹H NMR (500 MHz, DMSO-d₆) δ 12.85 (s, 2H), 11.07 (s, 1H), 7.23 (d, J=8.1 Hz, 1H), 7.16 (d, J=2.4 Hz, 1H), 7.04 (dd, J=7.9, 7.9 Hz, 1H), 6.61 (d, J=7.6 Hz, 1H), 6.56 (s, 2H), 2.89-2.76 (m, 2H), 2.73-2.60 (m, 4H), 2.32 (s, 6H), 2.14-2.04 (m, 1H), 1.22 (d, J=7.0 Hz, 3H), 1.02 (dd, J=21.7, 6.8 Hz, 6H).

### Example 12. Synthesis of compound 135: 3-(2-(dimethylamino)ethyl)-1H-indol-4-yl isopropyl(methyl)carbamate, 0.63Formic Acid, 0.37HCl

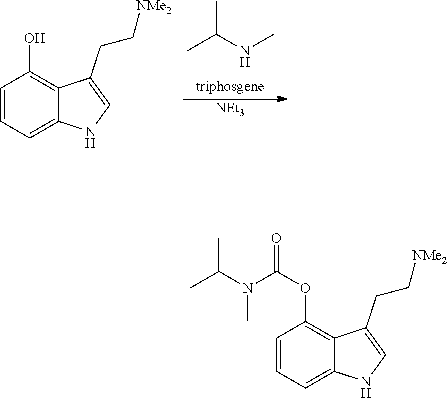

To a solution of N-methylpropan-2-amine (59 mg, 84 μL, 1.1 Eq, 808 μmol) and triethylamine (149 mg, 205 μL, 2 Eq, 1.47 mmol) in THF (4 mL) at 0° C. was added triphosgene (113 mg, 0.5 Eq, 367 μmol). The reaction was stirred at 0° C. for 20 min. A suspension of psilocin (150 mg, 1 Eq, 734 μmol) in THF (3 mL) was added at 0° C. The mixture was stirred at r.t. for 48 h. The reaction was stirred for a further 4 days at 40° C. The reaction was quenched with ice-cold water (10 mL) and diluted with ethyl acetate (10 mL). The phases were separated, and the aqueous phase was further extracted with ethyl acetate (10 mL). The combined organics were washed with brine (20 mL), dried (Na₂SO₄) and concentrated in vacuo. The crude material (211 mg) was dissolved in DMSO (2.7 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mLmin⁻¹ eluting with a 0.1% formic acid in water-MeCN gradient over 8.5 min using UV across all wavelengths with PDA as well as a QDA and ELS detector. At column dilution pump gives 2 mLmin⁻¹ MeCN over the entire method, which is included in the following MeCN percentages. Gradient information: 0.0-0.5 min, 7.5% MeCN; 0.5-5.5 min, ramped from 7.5% MeCN to 37.5% MeCN; 5.5-5.6 min, ramped from 37.5% MeCN to 100% MeCN; 5.6-8.5 min, held at 100% MeCN. The clean fractions were evaporated in a Genevac to afford the title compound (50.0 mg, 145 μmol, 20%) as a light brown solid.

m/z 304.2 (M+H)⁺ (ES+)

¹H NMR (500 MHz, DMSO-d₆) δ 11.03 (s, 1H), 8.15 (s, 0.63 H), 7.20 (dd, J=8.1, 0.8 Hz, 1H), 7.15 (d, J=2.3 Hz, 1H), 7.04-6.98 (m, 1H), 6.65-6.58 (m, 1H), 4.57-4.48 (m, 0.33H), 4.31 (p, J=6.6 Hz, 0.67H), 2.97 (s, 2H), 2.90-2.82 (m, 2H), 2.80 (s, 1H), 2.79-2.71 (m, 2H), 2.38 (s, 6H), 1.24 (d, J=6.7 Hz, 2H), 1.15 (d, J=6.8 Hz, 4H). 1× exchangeable H not observed.

### Example 13. Synthesis of compound 137: 3-(2-(Dimethylamino)ethyl)-1H-indol-4-yl isobutylcarbamate, HCl

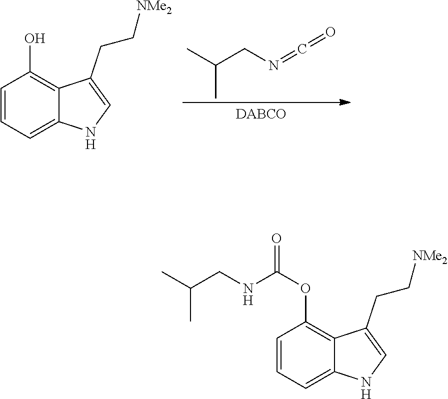

To a suspension of psilocin (205.0 mg, 88% Wt, 1 Eq, 883.1 μmol) in MeCN (5.00 mL) was added DABCO (118.9 mg, 1.2 Eq, 1.060 mmol) and 1-isocyanato-2-methylpropane, 1M in DCM (131.3 mg, 1.325 mL, 1.00 molar, 1.5 Eq, 1.325 mmol). The reaction was stirred at rt for 16 h. The volatiles were removed in vacuo. The crude product was loaded onto celite and purified by chromatography on RP Flash C18 (24 g cartridge, 0-20% (0.1% formic acid in MeCN)/(0.1% formic acid in water)) to afford an oil. The sample was triturated with acetone to afford the title compound (9.20 mg, 26 μmol, 2.3%, 95% Purity) as a dark brown solid.

m/z 304.2 (M+H)⁺ (ES+)

¹H NMR (500 MHz, DMSO) δ 11.21-11.10 (m, 1H), 10.98-10.10 (br s, 1H), 7.94 (m, 1H), 7.25-7.18 (m, 2H), 7.06-7.00 (m, 1H), 6.71 (d, J=7.6 Hz, 1H), 3.24-3.17 (m, 2H), 3.11-3.04 (m, 2H), 2.93 (t, J=6.4 Hz, 2H), 2.75 (s, 6H), 1.78 (hept, J=6.7 Hz, 1H), 0.91 (d, J=6.7 Hz, 6H).

### Example 14. Synthesis of compound 139: 3-(2-(dimethylamino)ethyl)-1H-indol-4-yl 2-isopropoxyacetate, Fumaric acid

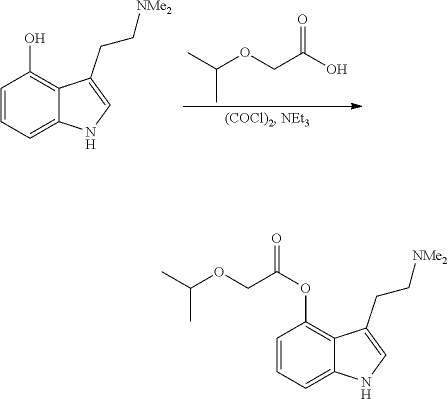

To a stirred solution of 2-(1-methylethoxy)-acetic acid (143 mg, 1.4 Eq, 1.21 mmol) in dry DCM (4 mL) under an atmosphere of nitrogen at room temp. was added oxalyl chloride (233 mg, 161 μL, 2.1 Eq, 1.84 mmol) and a drop of DMF. The reaction mixture was stirred at room temp for 2 h. The volatiles were removed in vacuo and the residue was dissolved in DCM (2 mL) and added to a solution of psilocin (179.0 mg, 1 Eq, 876 μmol) and triethylamine (323 mg, 445 μL, 3.6 Eq, 3.19 mmol) in DCM (2 mL) at 0° C. The reaction mixture was stirred at room temp for 1 h. The reaction mixture was diluted with water (5 mL). The aqueous layer was extracted with DCM (3×5 mL). The combined organic layers were collected, dried (Na₂SO₄), filtered and concentrated in vacuo. The crude product was purified by chromatography on RP Flash C18 (24 g cartridge, 5-50% (0.1% formic acid in MeCNY(0.1% formic acid in water)) to afford 3-(2-(dimethylamino)ethyl)-1H-indol-4-yl 2-isopropoxyacetate (187 mg, 614 μmol) as a brown oil. The material was dissolved in acetone (10 mL) and a solution of fumaric acid (71 mg, 0.7 Eq, 614 μmol) in acetone (10 mL) was added. The resulting solid was isolated by filtration, washed with acetone and dried in vacuo to afford the title compound (130 mg, 309 μmol, 35%) as an off-white solid.

m/z 305.2 (M+H)⁺ (ES+)

¹H NMR (500 MHz, DMSO-d₆) δ 11.10 (s, 1H), 7.25 (dd, J=8.1, 0.8 Hz, 1H), 7.18 (d, J=2.3 Hz, 1H), 7.09-7.02 (m, 1H), 6.72 (dd, J=7.6, 0.8 Hz, 1H), 6.56 (s, 2H), 4.48 (s, 2H), 3.75 (hept, J=6.0 Hz, 1H), 2.86-2.78 (m, 2H), 2.70-2.60 (m, 2H), 2.35 (s, 6H), 1.17 (d, J=6.1 Hz, 6H). (2× exchangeable H not observed).

### Example 15. Synthesis of compound 168: 3-(2-(dimethylamino)ethyl)-1H-indol-4-yl 3-bromobenzoate, Formic Acid

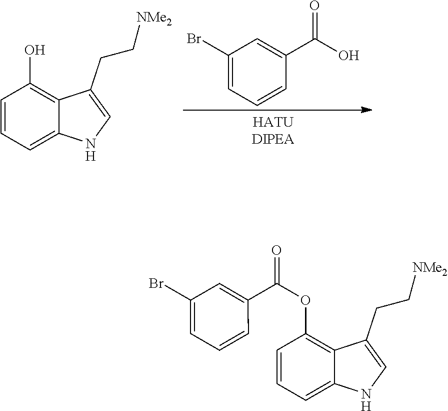 [SMILES: CN(C)CCc1c[nH]c2cccc(O)c12 ; O=C(O)c1cccc(Br)c1 ; CN(C)CCc1c[nH]c2cccc(OC(=O)c3cccc(Br)c3)c12]

A mixture of psilocin (200.0 mg, 1 Eq, 881 μmol), 3-bromobenzoic acid (181 mg, 1 Eq, 881 μmol), HATU (483 mg, 1.4 Eq, 1.27 mmol) and diisopropylethylamine (269 mg, 359 μL, 2.4 Eq, 2.08 mmol) in dry DMF (2.5 mL) was stirred at r.t. for 16 h. The reaction mixture was diluted with sat. aq. NaHCO₃(25 mL) and extracted with EtOAc (3×25 mL). The combined organic layers were washed with water (3×20 mL) and brine (25 mL), dried (Na₂SO₄), filtered, and concentrated in vacuo. The crude product was loaded onto silica and purified by chromatography on silica gel (12 g cartridge, 2-10% (0.7 M ammonia/MeOH)/DCM) to afford partially purified title compound. The material was loaded onto celite and purified by chromatography on RP Flash C18 (24 g cartridge, 5-50% (0.1% formic acid in MeCN)/(0.1% formic Acid in Water)) to afford the title compound (29.2 mg, 67 μmol, 8%) as a light brown solid.

m/z 387.1/389.0 (M+H)⁺ (ES+)

¹H NMR (500 MHz, DMSO-d₆) δ 11.21-11.10 (m, 1H), 8.36-8.29 (m, 1H), 8.26-8.17 (m, 2H), 8.03-7.95 (m, 1H), 7.63-7.57 (m, 1H), 7.31 (d, J=8.1 Hz, 1H), 7.20 (d, J=2.3 Hz, 1H), 7.14-7.08 (m, 1H), 6.83 (d, J=7.6 Hz, 1H), 2.78-2.70 (m, 2H), 2.61-2.55 (m, 2H), 2.10 (s, 6H). 1× exchangeable H not observed.

### Example 16. Synthesis of compound 169: 3-(2-(dimethylamino)ethyl)-1H-indol-4-yl 3-bromo-2-methylbenzoate, Fumaric acid

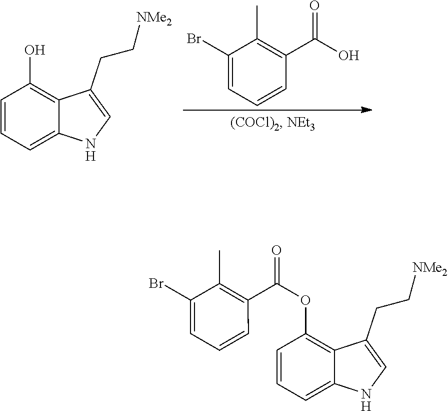 [SMILES: CN(C)CCc1c[nH]c2cccc(O)c12 ; Cc1c(Br)cccc1C(=O)O ; Cc1c(Br)cccc1C(=O)Oc1cccc2[nH]cc(CCN(C)C)c12]

To a stirred solution of 3-bromo-2-methylbenzoic acid (249 mg, 1.2 Eq, 1.16 mmol) in dry DCM (4 mL) under a nitrogen atmosphere at r.t. was added oxalyl dichloride (260 mg, 180 μL, 2.1 Eq, 2.05 mmol) and a drop of DMF. The reaction mixture was stirred at r.t. for 2 h. The volatiles were removed in vacuo. The residue was redissolved in DCM (2 mL) and added to a solution of psilocin (200.9 mg, 1 Eq, 964 μmol) and triethylamine (290 mg, 400 μL, 3 Eq, 2.87 mmol) in DCM (2 mL) at 0° C. The resulting mixture was stirred at r.t. for 1 h. The reaction mixture was diluted with water (5 mL), extracted with DCM (3×5 mL). The combined organic layers were collected, dried (Na₂SO₄), filtered and concentrated in vacuo. The crude product was purified by chromatography on silica gel (24 g cartridge, 0-20% McOH/DCM). The purified product was dissolved in acetone (15 mL). Fumaric acid (88 mg, 756 μmol) in acetone (20 mL) was added. The precipitate formed was collected by filtration to afford the title compound (283.2 mg, 0.54 mmol, 56%) as a white solid.

m/z 401.1/403.4 (M+H)⁺ (ES+)

¹H NMR (500 MHz, DMSO-d₆) δ 11.15 (s, 1H), 8.16 (dd, J=7.8, 1.3 Hz, 1H), 7.95 (dd, J=8.0, 1.3 Hz, 1H), 7.38 (dd, J=7.9 Hz, 1H), 7.31 (dd, J=8.1, 0.8 Hz, 1H), 7.20 (d, J=2.3 Hz, 1H), 7.11 (dd, J=7.9 Hz, 1H), 6.87 (dd, J=7.6, 0.8 Hz, 1H), 6.56 (s, 2H), 2.80-2.73 (m, 2H), 2.67 (s, 3H), 2.60 (t, J=8.1 Hz, 2H), 2.11 (s, 6H). 2× exchangeable protons not observed.

### Example 17. Synthesis of compound 171: 3-(2-(dimethylamino)ethyl)-1H-indol-4-yl 3-bromo-5-isopropylbenzoate, Fumaric acid

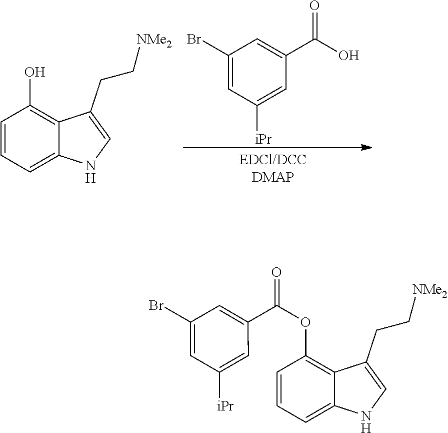 [SMILES: CN(C)CCc1c[nH]c2cccc(O)c12 ; CC(C)c1cc(Br)cc(C(=O)O)c1 ; CC(C)c1cc(Br)cc(C(=O)Oc2cccc3[nH]cc(CCN(C)C)c23)c1]

To a stirred solution of psilocin (79.4 mg, 1.1 Eq, 338 μmol), 3-bromo-5-isopropylbenzoic acid (76 mg, 1 Eq, 311 μmol) in dry DMF (2 mL) at r.t. was added DMAP (121 mg, 3.2 Eq, 990 μmol) and EDCI (91 mg, 1.5 Eq, 472 μmol). The reaction mixture was stirred at r.t. for 72 h. The reaction mixture was diluted with EtOAc (5 mL), washed with 5% citric acid solution (5 mL), water (5 mL) and sat. aq. NaHCO₃(5 mL), dried (Na₂SO₄), filtered and concentrated in vacuo. The crude product was purified by chromatography on silica gel (12 g cartridge, 0-10% MeOH/DCM) to afford a light brown oil. Separately, to a stirred solution of psilocin (124.2 mg, 1.1 Eq, 529 μmol) and 3-bromo-5-isopropylbenzoic acid (120 mg, 1 Eq, 494 μmol) in dry DMF (2.5 mL) at r.t. was added DMAP (102 mg, 1.7 Eq, 837 μmol) and DCC (181 mg, 157 μL, 1.8 Eq, 867 μmol). The reaction mixture was stirred at r.t. for 72 h. The reaction mixture was diluted with EtOAc (5 mL), washed with 5% citric acid solution (5 mL), water (5 mL) and sat. aq. NaHCO₃(5 mL), dried (Na₂SO₄), filtered and concentrated in vacuo. The crude product was purified by chromatography on silica gel (12 g cartridge, 0-10% MeOH/DCM) to afford a light brown oil.

The two oils were dissolved in acetone (1 mL) and combined. A solution of fumaric acid (7 mg, 0.20 Eq, 64 μmol) in acetone (1 mL) was added. The precipitate formed was collected by filtration to afford the title compound (12.2 mg, 22 μmol, 3%) as a beige solid.

m/z 429.5/431.6 (M+H)⁺ (ES+)

¹H NMR (500 MHz, DMSO-d₆) δ 11.16 (s, 1H), 8.14 (dd, J=1.7 Hz, 1H), 8.05 (dd, J=1.6 Hz, 1H), 7.87 (dd, J=1.8 Hz, 1H), 7.31 (dd, J=8.1, 0.8 Hz, 1H), 7.20 (d, J=2.4 Hz, 1H), 7.10 (dd, J=7.9 Hz, 1H), 6.83 (dd, J=7.7, 0.8 Hz, 1H), 6.55 (s, 2H), 3.11-3.02 (m, 1H), 2.79-2.72 (m, 2H), 2.66-2.59 (m, 2H), 2.12 (s, 6H), 1.26 (d, J=6.9 Hz, 6H). 2× exchangeable protons not observed.

### Example 18. Synthesis of compound 176: 3-(2-(dimethylamino)ethyl)-1H-indol-4-yl 2-phenylacetate, 0.8Fumaric acid, 0.2HCl

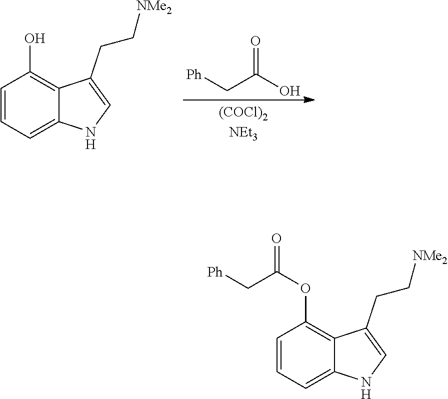 [SMILES: CN(C)CCc1c[nH]c2cccc(O)c12 ; O=C(O)Cc1ccccc1 ; CN(C)CCc1c[nH]c2cccc(OC(=O)Cc3ccccc3)c12]

To a stirred solution of 2-phenylacetic acid (117 mg, 1 Eq, 857 μmol) in dry DCM (4 mL) under an atmosphere of nitrogen at r.t. was added oxalyl chloride (228 mg, 158 μL, 2.1 Eq, 1.80 mmol) and a drop of DMF. The reaction mixture was stirred at r.t. for 2 h. The volatiles were removed in vacuo. The residue was dissolved in DCM (2 mL) and added to a solution of psilocin (198.9 mg, 1 Eq, 857 μmol) and triethylamine (312 mg, 430 μL, 3.6 Eq, 3.09 mmol) in DCM (2 mL) at 0° C. The reaction mixture was stirred at r.t. for 1 h. The reaction mixture was diluted with water (5 mL) and transferred into a separating funnel. The aqueous layer was extracted with DCM (3×5 mL). The combined organic layers were collected, dried (Na₂SO₄), filtered and concentrated in vacuo. The crude product was purified by chromatography on RP Flash C18 (24 g cartridge, 5-40% (0.1% formic acid in MeCN)/(0.1% formic acid in water)) to afford 3-(2-(dimethylamino)ethyl)-1H-indol-4-yl 2-phenylacetate, Formic Acid (84.6 mg, 0.18 mmol, 21%) as a brown oil. The partially purified material was dissolved in acetone (3 mL) and a solution of fumaric acid (21 mg, 0.21 Eq, 0.18 mmol) in acetone (4 mL) was added. The resulting solid was filtered, washed with acetone (2 mL) and dried in a vacuum desiccator for 24 h to afford the title compound (32.8 mg, 70 μmol, 8%) as a brown solid.

m/z 323.1 (M+H)⁺ (ES+)

¹H NMR (500 MHz, DMSO) δ 11.07 (s, 1H), 7.43-7.35 (m, 4H), 7.33-7.26 (m, 1H), 7.23 (d, J=8.1 Hz, 1H), 7.16 (d, J=2.3 Hz, 1H), 7.06-6.99 (m, 1H), 6.65 (d, J=7.6 Hz, 1H), 6.52 (s, 1.6H), 4.06 (s, 2H), 2.80 (t, J=7.8 Hz, 2H), 2.66-2.59 (m, 2H), 2.32 (s, 6H). (2× exchangeable H not observed)

### Example 19. Synthesis of compound 185: tert-Butyl 2-(3-((3-(2-(dimethylamino)ethyl)-1H-indol-4-yl)oxy)-3-oxopropyl)pyrrolidine-1-carboxylate, Fumaric acid

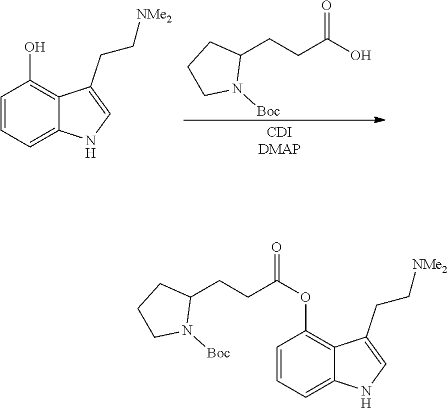 [SMILES: CN(C)CCc1c[nH]c2cccc(O)c12 ; CC(C)(C)OC(=O)N1CCCC1CCC(=O)O ; CN(C)CCc1c[nH]c2cccc(OC(=O)CCC3CCCN3C(=O)OC(C)(C)C)c12]

A solution of 3-(1-(tert-butoxycarbonyl)pyrrolidin-2-yl)propanoic acid (155 mg, 1.3 Eq, 636 μmol), DMAP (12.0 mg, 0.2 Eq, 97.9 μmol) and CDI (159 mg, 2 Eq, 979 μmol) was stirred at rt for 1 h. Psilocin (100 mg, 1 Eq, 490 μmol) was then added and the mixture was stirred at rt overnight. Another solution of 3-(1-(tert-butoxycarbonyl)pyrrolidin-2-yl)propanoic acid (119 mg, 1 Eq, 490 μmol), CDI (87.3 mg, 1.1 Eq, 539 μmol) and DMAP (5.98 mg, 0.1 Eq, 49.0 μmol) in THF (2.00 mL), previously stirred at rt for 1 h, was added to the mixture. The reaction mixture was left stirring at rt for 24 h. The mixture was heated at 40° C. for 24 h. The mixture was then heated at 55° C. over the weekend. The reaction mixture was cooled to rt, diluted with distilled water (10 mL) and transferred into a separating funnel. The organic layer was extracted with EtOAc (3×10 mL). The combined organic layers were washed with brine (1×15 mL), dried (Na₂SO₄), filtered and concentrated in vacuo. The crude product was purified by chromatography on RP Flash C18 (12 g cartridge, 5-50% (0.1% formic acid in MeCN)/(0.1% formic acid in water)) to afford the desired product (89.4 mg, 0.16 mmol, 33%, 85% Purity) as a brown oil. The material was dissolved in acetone (5 mL) and a solution of fumaric acid (19 mg, 0.33 Eq, 0.16 mmol) in acetone (5 mL) was added. The resulting solid was filtered, washed with acetone and dried in a vacuum desiccator over the weekend to afford the title compound (29.9 mg, 49.6 μmol, 10.1%, 90.6% Purity) as a brown solid.

m/z 430.36 (M+H)⁺ (ES+)

¹H NMR (500 MHz, DMSO) δ 11.10 (s, 1H), 7.23 (dd, J=8.1, 0.8 Hz, 1H), 7.18 (d, J=2.4 Hz, 1H), 7.08-7.01 (m, 1H), 6.73-6.66 (m, 1H), 6.57 (s, 2H), 3.81 (s, 1H, rotamer), 3.32-3.19 (m, 2H, rotamer), 2.86 (s, 1H, rotamer), 2.78 (s, 2H, rotamer), 2.70-2.65 (m, 1H, rotamer), 2.45 (s, 3H, rotamer), 2.42 (s, 3H, rotamer), 1.97-1.65 (m, 9H, rotamer), 1.40 (s, 9H). 2H are absent (exchangeable protons of fumaric acid).

### Example 20. Synthesis of compound 188: 3-(2-(dimethylamino)ethyl)-1H-indol-4-yl 2-(4-isobutylphenyl)propanoate, 0.75Fumaric acid

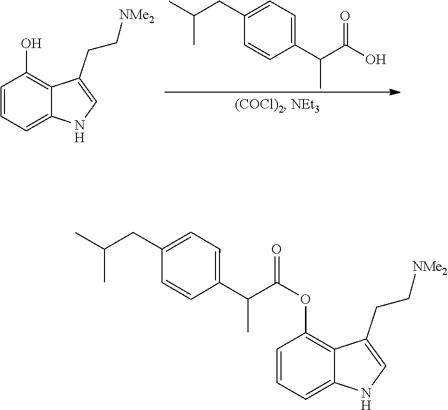 [SMILES: CN(C)CCc1c[nH]c2cccc(O)c12 ; CC(C)Cc1ccc(C(C)C(=O)O)cc1 ; CC(C)Cc1ccc(C(C)C(=O)Oc2cccc3[nH]cc(CCN(C)C)c23)cc1]

To a stirred solution of 2-(4-isobutylphenyl) propionic acid (124 mg, 1.2 Eq, 593 μmol) in dry DCM (4 mL) under a nitrogen atmosphere at r.t. was added oxalyl chloride (130 mg, 90 μL, 2.1 Eq, 1.02 mmol) and a drop of DMF. The reaction mixture was stirred at r.t. for 2 h. The volatiles were removed in vacuo. The residue was redissolved in DCM (2 mL) and added to a solution of psilocin (102.6 mg, 1 Eq, 492 μmol) and triethylamine (182 mg, 250 μL, 3.6 Eq, 1.79 mmol) in DCM (2 mL) at 0° C. The resulting mixture was stirred at r.t. for 1 h. The reaction mixture was diluted with water (5 mL), extracted with DCM (3×5 mL). The combined organic layers were collected, dried (Na₂SO₄), filtered and concentrated in vacuo. The crude product was purified by chromatography on silica gel (12 g cartridge, 0-20% MeOH/DCM) to afford a light brown oil. The product was dissolved in acetone (2 mL). fumaric acid (34 mg, 293 μmol) in acetone (2 mL) was added. The precipitate formed was collected by filtration to afford the title compound (51.4 mg, 0.11 mmol, 22%) as a beige solid.

m/z 393.3 (M+H)⁺ (ES+)

¹H NMR (500 MHz, DMSO-d₆) δ 11.04 (s, 1H), 7.36-7.32 (m, 2H), 7.23-7.16 (m, 3H), 7.14 (d, J=2.3 Hz, 1H), 7.00 (t, J=7.9 Hz, 1H), 6.55 (s, 1.5H), 6.50 (dd, J=7.6, 0.8 Hz, 1H), 4.13 (q, J=7.1 Hz, 1H), 2.72-2.65 (m, 2H), 2.61-2.52 (m, 2H), 2.45 (d, J=7.1 Hz, 2H), 2.28 (s, 6H), 1.83 (dq, J=13.5, 6.7 Hz, 1H), 1.55 (d, J=7.1 Hz, 3H), 0.87 (d, J=6.6 Hz, 6H). 1.5× exchangeable protons not observed.

### Example 21. Synthesis of compound 189: 3-(2-(dimethylamino)ethyl)-1H-indol-4-yl ((trimethylsilyl)methyl) carbonate, Formic Acid

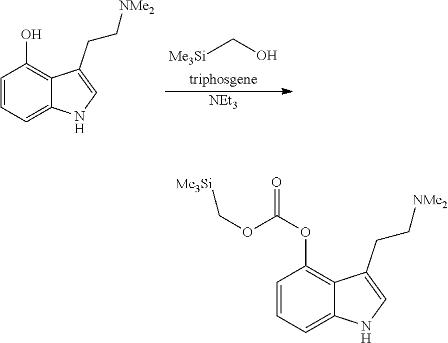 [SMILES: CN(C)CCc1c[nH]c2cccc(O)c12 ; C[Si](C)(C)CO ; CN(C)CCc1c[nH]c2cccc(OC(=O)OC[Si](C)(C)C)c12 ; "NEt3"]

To a solution of (trimethylsilyl)methanol (148 mg, 1.3 Eq, 1.42 mmol) and triethylamine (174 mg, 240 μL, 1.6 Eq, 1.72 mmol) in THF (6 mL) at 0° C. was added triphosgene (160 mg, 0.5 Eq, 518 μmol). The reaction was stirred for 3 h at 0° C. A suspension of psilocin (227.2 mg, 1 Eq, 1.11 mmol) and triethylamine (145 mg, 200 μL, 1.3 Eq, 1.43 mmol) in THF (5 mL) was added at 0 C. The mixture was stirred at 0° C. for 30 min then at r.t. for 40 h. The reaction was quenched with ice-cold water (10 mL) and diluted with ethyl acetate (10 mL). The phases were separated and the aqueous phase was further extracted with ethyl acetate (10 mL). The combined organics were washed with brine (20 mL), dried (Na₂SO₄) and concentrated in vacuo. The crude product was purified by chromatography on RP Flash C18 (4 g cartridge, 5-50% (0.1% formic acid in MeCN)/0.1% formic acid in water)) (eluting ˜25%) to afford the title compound (12.4 mg, 31 μmol, 3%) as a sticky brown gum after trituration with diethyl ether (3×5 mL).

m/z 335.2 (M+H)⁺ (ES+)

¹H NMR (500 MHz, DMSO-d₆) δ 11.07 (s, 1H), 8.19 (s, 1H), 7.25 (d, J=8.1 Hz, 1H), 7.17 (d, J=2.3 Hz, 1H), 7.04 (dd, J=7.9, 7.9 Hz, 1H), 6.73 (d, J=7.6 Hz, 1H), 3.95 (s, 2H), 2.81-2.72 (m, 2H), 2.48 (s, 2H), 2.21 (s, 6H), 0.10 (s, 9H). 1× exchangeable H not observed Example 22. Synthesis of compound 191: 3-(2-(dimethylamino)ethyl)-1H-indol-4-yl (3-(trimethylsilyl)propyl) carbonate, Formic Acid

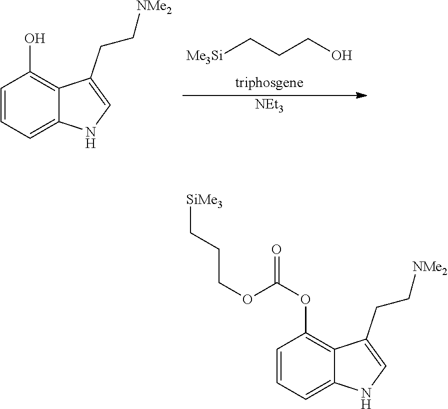 [SMILES: CN(C)CCc1c[nH]c2cccc(O)c12 ; C[Si](C)(C)CCCO ; CN(C)CCc1c[nH]c2cccc(OC(=O)OCCC[Si](C)(C)C)c12 ; "NEt3"]

To a solution of 3-(trimethylsilyl)propan-1-ol (179 mg, 1.1 Eq, 1.35 mmol) and triethylamine (174 mg, 240 μL, 1.4 Eq, 1.72 mmol) in THF (6 mL) at 0° C. was added triphosgene (189 mg, 0.5 Eq, 612 μmol). The reaction was stirred for 20 min at 0° C. A suspension of psilocin (250.8 mg, 1 Eq, 1.23 mmol) in THF (5 mL) was added at 0° C. The mixture was stirred at r.t. for 16 h. The reaction was quenched with ice-cold water (10 mL) and diluted with ethyl acetate (10 mL). The phases were separated, and the aqueous phase was further extracted with ethyl acetate (10 mL). The combined organics were washed with brine (20 mL), dried (Na₂SO₄) and concentrated in vacuo. The crude product was partially purified by chromatography on silica gel (4 g cartridge, 0-10% (0.7 M Ammonia/MeOH)/DCM) (eluting ˜2%). The partially purified crude product was purified by chromatography on RP Flash C18 (4 g cartridge, 10-100% (0.1% Formic acid in MeCN)/(0.1% Formic Acid in Water)) (eluting ˜25%) to afford the title compound (92.0 mg, 0.22 mmol, 18%) as an off-white solid after trituration with diethyl ether (2×5 mL).

m/z 363.2 (M+H)⁺ (ES+)

¹H NMR (500 MHz, DMSO-d₆) δ 11.09 (s, 1H), 8.17 (s, 1H), 7.26 (d, J=8.1 Hz, 1H), 7.18 (d, J=2.3 Hz, 1H), 7.04 (dd, J=7.9, 7.9 Hz, 1H), 6.75 (d, J=7.6 Hz, 1H), 4.17 (t, J=6.8 Hz, 2H), 2.83-2.73 (m, 2H), 2.55-2.51 (m, 2H), 2.23 (s, 6H), 1.72-1.60 (m, 2H), 0.61-0.48 (m, 2H), 0.01 (s, 9H). 1× exchangeable H not observed.

### Example 23. Synthesis of compound 195: 1-(3-(2-(dimethylamino)ethyl)-4-hydroxy-1H-indol-1-yl)-3-methylbutan-1-one, Formic Acid

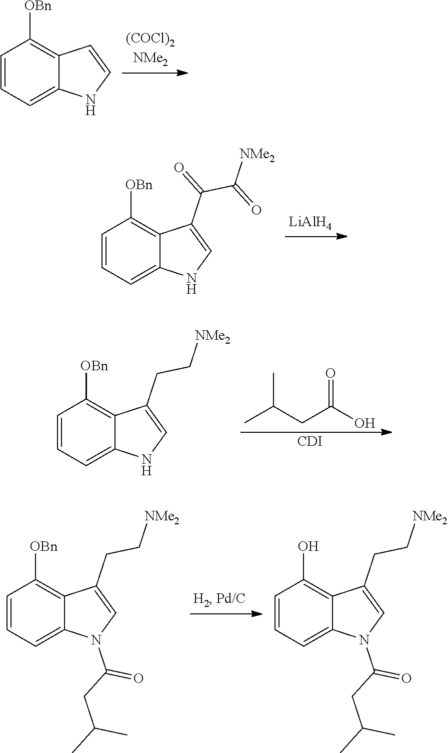

### Step 1: 2-(4-(benzyloxy)-1H-indol-3-yl)-N,N-dimethyl-2-oxoacetamide

Oxalyl chloride (3.13 g, 2.2 mL, 1.1 Eq, 24.6 mmol) was added portion-wise to a stirred solution of 4-(benzyloxy)-1H-indole (5.00 g, 1 Eq, 22.4 mmol) in MTBE (100 mL) and stirred at r.t. for 90 min. The reaction mixture was then cooled to 0° C. and dimethylamine in THF (5.55 g, 61.6 mL, 2.0 M, 5.5 Eq, 123 mmol) added portion-wise over 5 min. The reaction mixture was left in the ice-bath which was allowed to warm slowly to r.t. over the course of another 90 min. The resultant suspension was filtered and the filtered solid partitioned between EtOAc (300 ml) and water (200 mL). The organic layer was separated, washed with sat. aq. NaHCO; (200 ml) and concentrated in vacuo to afford 2-(4-(benzyloxy)-1H-indol-3-yl)-N,N-dimethyl-2-oxoacetamide (4.76 g, 14.8 mmol, 66%) as a beige foam.

m/z 323.5 (M+H)⁺ (ES+)

¹H NMR (500 MHz, DMSO-d₆) δ 12.26 (s, 1H), 8.06 (s, 1H), 7.64-7.58 (m, 2H), 7.37 (dd, J=8.3, 6.9 Hz, 2H), 7.28 (t, J=7.3 Hz, 1H), 7.14-7.06 (m, 2H), 6.69 (dd, J=7.2, 1.5 Hz, 1H), 5.26 (s, 2H), 2.90 (d, J=16.8 Hz, 6H).

### Step 2: 2-(4-(benzyloxy)-1H-indol-3-yl)-N,N-dimethylethan-1-amine

Lithium Aluminium hydride in THF (1.96 g, 21.5 mL, 2.4 molar, 3.5 Eq, 51.7 mmol) was added portion-wise to stirred suspension of the product from step 1 above (4.76 g, 1 Eq, 14.8 mmol) in 2-MeTHF (100 mL) at 0° C. and under nitrogen. The mixture was left to stir as the ice-bath warmed to r.t. for 30 min before being stirred at 80° C. for 4 h. The reaction mixture was allowed to cool to r.t. before sodium sulfate decahydrate was added portionwise until the resultant effervescence subsided. The reaction mixture was filtered, the solid plug washed with MeOH (30 mL) and the clear filtrate collected allowed to stand at r.t. overnight. The resulting dark green solution was concentrated under reduced pressure to afford a dark green oil which solidified on standing. The solid was partitioned between EtOAc (200 mL) and water (100 mL). The organic layer was separated and concentrated under vacuum to afford the sub-tide compound (4.16 g, 13 mmol, 90%) as a brown solid which solidified on standing.

m/z 295.2 (M+H)⁺ (ES+)

¹H NMR (500 MHz, DMSO-d₆) δ 10.75 (s, 1H), 7.56-7.50 (m, 2H), 7.39 (dd, J=8.3, 6.8 Hz, 2H), 7.36-7.29 (m, 1H), 6.97 (d, J=2.3 Hz, 1H), 6.96-6.89 (m, 2H), 6.53 (dd, J=6.0, 2.5 Hz, 1H), 5.16 (s, 2H), 2.94-2.87 (m, 2H), 2.50-2.42 (m, 2H), 2.05 (s, 6H).

Step 3: 1-(4-(benzyloxy)-3-(2-(dimethylamino)ethyl)-1H-indol-1-yl)-3-methylbutan-1-one

CDI (214 mg, 1.7 Eq, 1.32 mmol) was added to solution of 3-methylbutanoic acid (122 mg, 1.5 Eq, 1.20 mmol) in DCM (3.00 mL) and stirred at for 2 h. Separately, potassium tert-butoxide (148 mg, 1.7 Eq, 1.32 mmol) was added to a solution of the product from step 2 above (250 mg, 1 Eq, 798 μmol) in DMF (5 mL) and stirred at r.t. for 90 min. The DCM solution was added to the DMF solution and left to stir at r.t. for 20 h. The reaction mixture was a partitioned between EtOAc (20 mL) and brine (20 mL). The organic layer was separated and concentrated in vacuo. The crude product was purified by chromatography on RP Flash C18 (12 g cartridge, 0-100% (0.1% formic acid in MeCN)/(0.1% formic acid in water)) to afford the sub-title compound (102 mg, 269 μmol, 34%) as a colourless gum.

m/z 379.2 (M+H)⁺ (ES+)

### Step 4: 1-(3-(2-(dimethylamino)ethyl)-4-hydroxy-1H-indol-1-yl)-3-methylbutan-1-one, Formic Acid

The product from step 3 above (102 mg, 1 Eq, 256 μmol) in methanol (10 mL) was passed through a 10/a Pd/C CatCart (H-cube, recirculation) at 1 mL/min, 60° C. and at 1 bar for 90 min. The bulk solvent was removed in vacuo. The crude product was dissolved in DMSO (2 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mLmin⁻¹ eluting with a 0.1% formic acid in water-MeCN gradient over 8.5 min using UV across all wavelengths with PDA as well as a QDA and ELS detector. At column dilution pump gives 2 mLmin⁻¹ MeCN over the entire method, which is included in the following MeCN percentages. Gradient information. 0.0-0.5 min, 12.5% MeCN; 0.5-5.5 min, ramped from 12.5% MeCN to 42.5% MeCN; 5.5-5.6 min, ramped from 42.5% MeCN to 100% MeCN; 5.6-8.5 min, held at 100% MeCN. The clean fractions were evaporated in a Genevac to afford the title compound (37.0 mg, 0.11 mmol, 43%) as colourless flakes.

m/z 289.2 (M+H)⁺ (ES+)

¹H NMR (500 MHz, DMSO-d₆) δ 8.19 (s, 1H), 7.82 (d, J=8.1 Hz, 1H), 7.53 (s, 1H), 7.08-7.05 (m, 1H), 6.61 (d, J=7.8 Hz, 1H), 2.92 (t, J=7.1 Hz, 2H), 2.82 (d, J=7.0 Hz, 2H), 2.69 (t, J=7.1 Hz, 2H), 2.30 (s, 6H), 2.18 (dq, J=13.4, 6.7 Hz, 1H), 0.99 (d, J=6.7 Hz, 6H). (2× exchangeable H not observed).

### Example 24. Synthesis of compound 197: ethyl 3-(2-(dimethylamino)ethyl)-4-hydroxy-1H-indole-1-carboxylate, Formic Acid

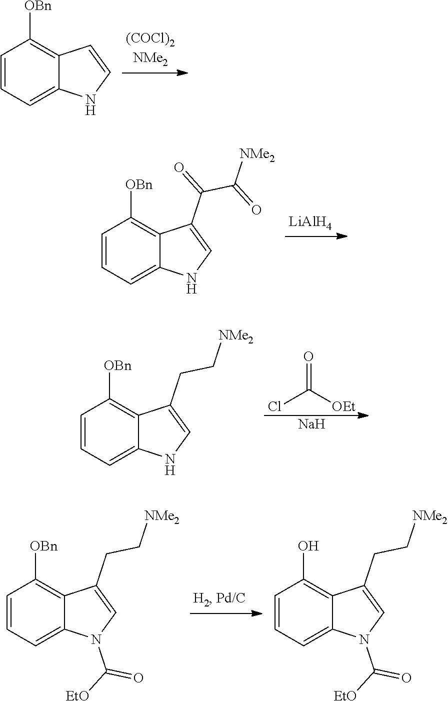

### Step 1: 2-(4-(benzyloxy)-1H-indol-3-yl)-N,N-dimethyl-2-oxoacetamide

Oxalyl chloride (3.13 g, 2.2 mL, 1.1 Eq, 24.6 mmol) was added portion-wise to a stirred solution of 4-(benzyloxy)-1H-indole (5.00 g, 1 Eq, 22.4 mmol) in MTBE (100 mL) and stirred at r.t. for 90 min. The reaction mixture was then cooled to 0° C. and dimethylamine in THF (5.55 g, 61.6 mL, 2.0 M, 5.5 Eq, 123 mmol) added portion-wise over 5 min. The reaction mixture was left in the ice-bath which was allowed to warm slowly to r.t. over the course of another 90 min. The resultant suspension was filtered and the filtered solid partitioned between EtOAc (300 ml) and water (200 mL). The organic layer was separated, washed with sat. aq. NaHCO₃(200 ml) and concentrated in vacuo to afford 2-(4-(benzyloxy)-1H-indol-3-yl)-N,N-dimethyl-2-oxoacetamide (4.76 g, 14.8 mmol, 66%) as a beige foam.

m/z 323.5 (M+H)⁺ (ES+)

¹H NMR (500 MHz, DMSO-d₆) δ 12.26 (s, 1H), 8.06 (s, 1H), 7.64-7.58 (m, 2H), 7.37 (dd, J=8.3, 6.9 Hz, 2H), 7.28 (t, J=7.3 Hz, 1H), 7.14-7.06 (m, 2H), 6.69 (dd, J=7.2, 1.5 Hz, 1H), 5.26 (s, 2H), 2.90 (d, J=16.8 Hz, 6H).

### Step 2: 2-(4-(benzyloxy)-1H-indol-3-yl)-N,N-dimethylethan-1-amine

Lithium Aluminium hydride in THF (1.96 g, 21.5 mL, 2.4 molar, 3.5 Eq, 51.7 mmol) was added portion-wise to stirred suspension of the product from step 1 above (4.76 g, 1 Eq, 14.8 mmol) in 2-MeTHF (100 mL) at 0° C. and under nitrogen. The mixture was left to stir as the ice-bath warmed to r.t. for 30 min before being stirred at 80° C. for 4 h. The reaction mixture was allowed to cool to r.t. before sodium sulfate decahydrate was added portionwise until the resultant effervescence subsided. The reaction mixture was filtered, the solid plug washed with MeOH (30 mL) and the clear filtrate collected allowed to stand at r.t. overnight. The resulting dark green solution was concentrated under reduced pressure to afford a dark green oil which solidified on standing. The solid was partitioned between EtOAc (200 mL) and water (100 mL). The organic layer was separated and concentrated under vacuum to afford the sub-title compound (4.16 g, 13 mmol, 90%) as a brown solid which solidified on standing.

m/z 295.2 (M+H)⁺ (ES+)

¹H NMR (500 MHz, DMSO-d₆) δ 10.75 (s, 1H), 7.56-7.50 (m, 2H), 7.39 (dd, J=8.3, 6.8 Hz, 2H), 7.36-7.29 (m, 1H), 6.97 (d, J=2.3 Hz, 1H), 6.96-6.89 (m, 2H), 6.53 (dd, J=6.0, 2.5 Hz, 1H), 5.16 (s, 2H), 2.94-2.87 (m, 2H), 2.50-2.42 (m, 2H), 2.05 (s, 6H).

### Step 3: ethyl 3-(2-(dimethylamino)ethyl)-4-hydroxy-1H-indole-1-carboxylate, Formic Acid

To a solution of the product from step 2 above (53.9 mg, 1 Eq, 183 μmol) in DMF (1 mL) at 0° C. was added sodium hydride (9 mg, 60% Wt, 1.2 Eq, 220 μmol) and the reaction was stirred for 10 min at 0° C. Ethyl chloroformate (34 mg, 30 μL, 1.7 Eq, 312 μmol) was added at 0° C. then stirred at r.t. for 22 h.

In a separate vial, to a solution of the product from step 1 above (252.9 mg, 1 Eq, 859 μmol) in DMF (5 mL) at 0° C. was added sodium hydride (48.0 mg, 1.4 Eq, 1.20 mmol) and the reaction was stirred for 10 min at 0° C. Ethyl chloroformate (170 mg, 150 μL, 1.8 Eq, 1.56 mmol) was added at 0° C. then stirred at r.t. for 18 h. The mixture was combined with the mixture above, diluted with ethyl acetate (15 mL), and poured into water/brine (4:1, 50 mL). The phases were separated and the aqueous was further extracted with ethyl acetate (20 mL). The combined organics were washed with brine (50 mL), dried (Na₂SO₄) and concentrated in vacuo. The crude product was purified by chromatography on RP Flash C18 (12 g cartridge, 5-50% (0.1% formic acid in MeCN)/(0.1% formic acid in water)) (eluting 20%) to afford the sub-title compound (163 mg, 0.34 mmol, 32%) as a clear yellow oil.

m/z 367.3 (M+H)⁺ (ES+)

¹H NMR (500 MHz, DMSO-d₆) δ 8.20 (s, 1H), 7.71 (d, J=8.3 Hz, 1H), 7.57-7.51 (m, 2H), 7.45-7.31 (m, 4H), 7.25 (dd, J=8.2, 8.2 Hz, 1H), 6.90 (d, J=8.1 Hz, 1H), 5.22 (s, 2H), 4.42 (q, J=7.1 Hz, 2H), 2.99-2.89 (m, 2H), 2.69 (q, J=5.5 Hz, 2H), 2.19 (s, 6H), 1.38 (t, J=7.1 Hz, 3H) (lx exchangeable H not observed).

### Step 4: ethyl 3-(2-(dimethylamino)ethyl)-4-hydroxy-1H-indole-1-carboxylate, Formic Acid

A solution of the product from step 3 above (163 mg, 1 Eq, 395 μmol) in Ethanol (5 mL) was a hydrogenated in the H-Cube (10% Pd/C cat cart, Batch: N0755, ID, THS-01131) at 5 bar, 1 mL/min for 1 h (recirculation). The solution was then hydrogenated at 15 bar, 1 mL/min for 1 h (recirculation). The collected mixture was concentrated in vacuo. The resulting material was triturated with acetonitrile (5 mL) and diethyl ether (5 mL) to afford the title compound (78.0 mg, 0.24 mmol, 60%) as a white solid.

m/z 277.2 (M+H)⁺ (ES+)

¹H NMR (500 MHz, DMSO-d₆) δ 11.95 (br, 1H), 8.19 (s, 1H), 7.54 (d, J=8.2 Hz, 1H), 7.34 (s, 1H), 7.08 (dd, J=8.1, 8.1 Hz, 1H), 6.59 (d, J=7.9 Hz, 1H), 4.39 (q, J=7.1 Hz, 2H), 2.92 (t, J=7.0 Hz, 2H), 2.66 (t, J=7.0 Hz, 2H), 2.28 (d, J=1.5 Hz, 6H), 1.37 (t, J=7.1 Hz, 3H) (1× exchangeable H not observed).

### Example 25. Synthesis of compound 199: isobutyl 3-(2-(dimethylamino)ethyl)-4-hydroxy-1H-indole-1-carboxylate, 0.5Fumaric acid

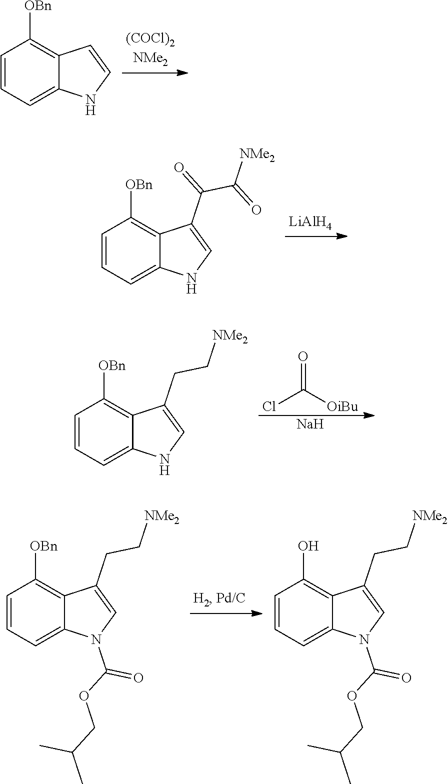

### Step 1: 2-(4-(benzyloxy)-1H-indol-3-yl)-N,N-dimethyl-2-oxoacetamide

Oxalyl chloride (3.13 g, 2.2 mL, 1.1 Eq, 24.6 mmol) was added portion-wise to a stirred solution of 4-(benzyloxy)-1H-indole (5.00 g, 1 Eq, 22.4 mmol) in MTBE (100 mL) and stirred at r.t. for 90 min. The reaction mixture was then cooled to 0° C. and dimethylamine in THF (5.55 g, 61.6 mL, 2.0 M, 5.5 Eq, 123 mmol) added portion-wise over 5 min. The reaction mixture was left in the ice-bath which was allowed to warm slowly to r.t. over the course of another 90 min. The resultant suspension was filtered and the filtered solid partitioned between EtOAc (300 ml) and water (200 mL). The organic layer was separated, washed with sat. aq. NaHCO₃(200 ml) and concentrated in vacuo to afford 2-(4-(benzyloxy)-1H-indol-3-yl)-N,N-dimethyl-2-oxoacetamide (4.76 g, 14.8 mmol, 66%) as a beige foam.

m/z 323.5 (M+H)⁺ (ES+)

¹H NMR (500 MHz, DMSO-d₆) δ 12.26 (s, 1H), 8.06 (s, 1H), 7.64-7.58 (m, 2H), 7.37 (dd, J=8.3, 6.9 Hz, 2H), 7.28 (t, J=7.3 Hz, 1H), 7.14-7.06 (m, 2H), 6.69 (dd, J=7.2, 1.5 Hz, 1H), 5.26 (s, 2H), 2.90 (d, J=16.8 Hz, 6H).

### Step 2: 2-(4-(benzyloxy)-1H-indol-3-yl)-N,N-dimethylethan-1-amine

Lithium Aluminium hydride in THF (1.96 g, 21.5 mL, 2.4 molar, 3.5 Eq, 51.7 mmol) was added portion-wise to stirred suspension of the product from step 1 above (4.76 g, 1 Eq, 14.8 mmol) in 2-MeTHF (100 mL) at 0° C. and under nitrogen. The mixture was left to stir as the ice-bath warmed to r.t. for 30 min before being stirred at 80° C. for 4 h. The reaction mixture was allowed to cool to r.t. before sodium sulfate decahydrate was added portionwise until the resultant effervescence subsided. The reaction mixture was filtered, the solid plug washed with MeOH (30 mL) and the clear filtrate collected allowed to stand at r.t. overnight. The resulting dark green solution was concentrated under reduced pressure to afford a dark green oil which solidified on standing. The solid was partitioned between EtOAc (200 mL) and water (100 mL). The organic layer was separated and concentrated under vacuum to afford the sub-title compound (4.16 g, 13 mmol, 90%) as a brown solid which solidified on standing.

m/z 295.2 (M+H)⁺ (ES+)

¹H NMR (500 MHz, DMSO-d₆) δ 10.75 (s, 1H), 7.56-7.50 (m, 2H), 7.39 (dd, J=8.3, 6.8 Hz, 2H), 7.36-7.29 (m, 1H), 6.97 (d, J=2.3 Hz, 1H), 6.96-6.89 (m, 2H), 6.53 (dd, J=6.0, 2.5 Hz, 1H), 5.16 (s, 2H), 2.94-2.87 (m, 2H), 2.50-2.42 (m, 2H), 2.05 (s, 6H).

### Step 3: isobutyl 4-(benzyloxy)-3-(2-(dimethylamino)ethyl)-1H-indole-1-carboxylate, Formic Acid

To a solution of 2-(4-(benzyloxy)-1H-indol-3-yl)-N,N-dimethylethan-1-amine (257.5 mg, 1 Eq, 875 μmol) in DMF (5 mL) at 0° C. was added sodium hydride (45 mg, 60% Wt, 1.3 Eq, 1.13 mmol) and the reaction was stirred for 10 min at 0° C. isobutyl carbonochloridate (263 mg, 250 μL, 2.2 Eq, 1.93 mmol) was added at 0° C. then stirred at r.t. for 2 h. The mixture was diluted with ethyl acetate (20 mL). Water/brine (1:1, 50 mL) was added and the phases were separated. The aqueous phase was extracted with EtOAc (20 mL). The combined organics were washed sequentially with water/brine (1:1, 50 mL), brine (50 mL), dried (Na₂SO₄) and concentrated in vacuo. The crude product was purified by chromatography on RP Flash C18 (24 g cartridge, 5-50% (0.1% formic acid in MeCN)/(0.1% formic acid in water)) to afford the sub-title compound (161 mg, 0.30 mmol, 35%) as a thick brown oil.

ml: 395.3 (M+H)⁺ (ES+)

¹H NMR (500 MHz, DMSO-d₆) δ 8.19 (s, 1H), 7.70 (d, J=8.3 Hz, 1H), 7.55-7.50 (m, 2H), 7.46-7.39 (m, 3H), 7.38-7.32 (m, 1H), 7.25 (dd, J=8.2, 8.2 Hz, 1H), 6.91 (d, J=8.1 Hz, 1H), 5.23 (s, 2H), 4.18 (d, J=6.5 Hz, 2H), 3.01-2.93 (m, 2H), 2.73-2.64 (m, 2H), 2.19 (s, 6H), 2.12-2.03 (m, 1H), 1.00 (d, J=6.7 Hz, 6H). (1× exchangeable H not observed).

### Step 4: isobutyl 3-(2-(dimethylamino)ethyl)-4-hydroxy-1H-indole-1-carboxylate, 0.5Fumaric acid

A solution of the product from step 3 above (161 mg, 1 Eq, 303 μmol) in ethanol (5 mL) was a hydrogenated in the H-Cube (Pd/C cat cart) at 1 bar at 60° C., 1 mL/min for 90 min (recirculation). The mixture was concentrated in vacuo. The crude product was purified by chromatography on RP Flash C18 (12 g cartridge, 5-50% (0.1% formic acid in MeCN)/(0.1% formic acid in water)) to afford isobutyl 3-(2-(dimethylamino)ethyl)-4-hydroxy-1H-indole-1-carboxylate, formic acid (47.0 mg, 134 μmol, 44%) as a pale brown oil. To a solution of the oil in acetone (2 mL) was added a solution of fumaric acid (17 mg, 1.1 Eq, 146 μmol) in acetone (3 mL). The mixture was chilled at −20° C. for 1 h. The resulting solid was isolated by filtration and dried in vacuo to afford the title compound (30.1 mg, 81 μmol, 61%) as a white solid.

m/z 305.2 (M+H)⁺ (ES+)

¹H NMR (500 MHz, DMSO-d₆) δ 11.62 (1H, br), 7.53 (d, J=8.2 Hz, 1H), 7.37 (s, 1H), 7.09 (dd, J=8.1, 8.1 Hz, 1H), 6.60 (d, J=7.8 Hz, 1H), 6.53 (s, 1H), 4.16 (d, J=6.5 Hz, 2H), 2.94 (t, J=7.0 Hz, 2H), 2.74-2.68 (m, 2H), 2.32 (s, 6H), 2.12-2.02 (m, 1H), 1.00 (d, J=6.7 Hz, 6H). (1× exchangeable H not observed).

### Example 26. Synthesis of compound 52: Ethyl 3-(2-(dimethylamino)ethyl)-4-((ethoxycarbonyl)oxy)-1H-indole-1-carboxylate

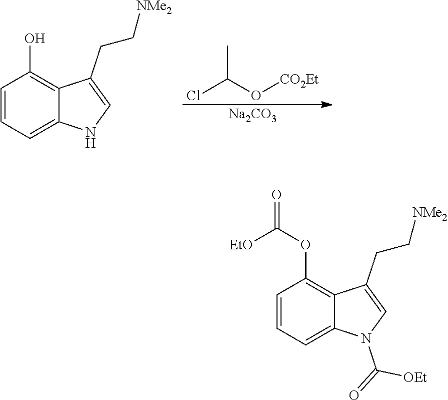

To a suspension of 3-(2-(dimethylamino)ethyl)-1H-indol-4-ol (25.0 mg, 99% Wt, 1 Eq, 121 μmol) and Na₂CO₃ (128 mg, 10 Eq, 1.21 mmol) in dry MeCN (1.00 mL) at 0° C. was added 1-chloroethyl ethyl carbonate (21.3 mg, 18.7 μL, 1.15 Eq, 139 μmol). The reaction mixture was allowed to warm to rt, and then stirred at 50° C. for 72 h. The reaction mixture was poured into water (20 mL) and diluted with EtOAc (20 mL). The organic layer was collected, and the aqueous layer was extracted with EtOAc (2×10 mL). The combined extracted layers were collected, dried (MgSO₄), filtered and concentrated in vacuo. The crude product was purified by chromatography on silica gel (4 g cartridge, 0-10/6 EtOAc/isohexane) to afford the title compound (20.5 mg, 51 μmol, 42%, 87% Purity) as a yellow oil.

m/z 349.07 (M+H)⁺ (ES+)

¹H NMR (500 MHz, CDCl3) δ 8.08 (d, J=8.3 Hz, 1H), 7.40 (s, 1H), 7.30 (t, J=8.2 Hz, 1H), 7.06 (dd, J=8.0, 0.8 Hz, 1H), 4.47 (q, J=7.1 Hz, 2H), 4.35 (q, J=7.1 Hz, 2H), 2.93-2.86 (m, 2H), 2.65-2.59 (m, 2H), 2.32 (s, 6H), 1.46 (t, J=7.1 Hz, 3H), 1.40 (t, J=7.1 Hz, 3H).

### Example 27. Synthesis of compound 78: 3-(2-(dimethylamino)ethyl)-1H-indol-4-yl 2-(4-chlorophenoxy)-2-methylpropanoate, Fumaric acid

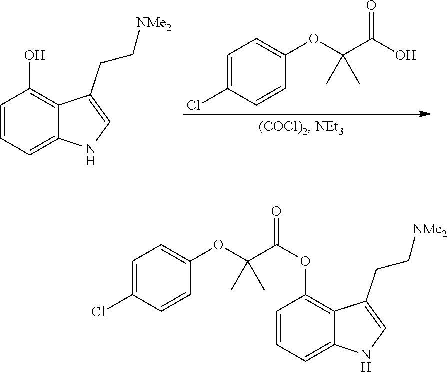 [SMILES: CN(C)CCc1c[nH]c2cccc(O)c12 ; CC(C)(Oc1ccc(Cl)cc1)C(=O)O ; CN(C)CCc1c[nH]c2cccc(OC(=O)C(C)(C)Oc3ccc(Cl)cc3)c12]

To a stirred solution of Clofibric acid (381 mg, 1.4 Eq, 1.78 mmol) in dry DCM (5 mL) under an atmosphere of nitrogen at room temp. was added oxalyl chloride (338 mg, 233 μL, 2.1 Eq, 2.67 mmol) and a drop of DMF. The reaction mixture was stirred at room temp for 2 h. The volatiles were removed in vacuo. The residue was dissolved in DCM (3 mL) and added to a solution of psilocin (259.3 mg, 1 Eq, 1.27 mmol) and triethylamine (462 mg, 637 μL, 3.6 Eq, 4.57 mmol) in DCM (3 mL) at 0° C. The reaction mixture was stirred r.t. for 1 h. The reaction mixture was diluted with water (5 mL) and transferred into a separating funnel. The aqueous layer was extracted with DCM (3×5 mL). The combined organic layers were collected, dried (Na₂SO₄), filtered and concentrated in vacuo. The crude product was purified by chromatography on RP Flash C18 (24 g cartridge, 5-50% (0.1% formic acid in MeCN)/(0.1% formic acid in water)) to afford 3-(2-(dimethylamino)ethyl)-1H-indol-4-yl 2-(4-chlorophenoxy)-2-methylpropanoate, Formic Acid (322 mg, 720 μmol, 57%) as a brown oil. The material was dissolved in acetone (10 mL) and a solution of fumaric acid (84 mg, 0.720 mmol) in acetone (10 mL) was added. The resulting solid was filtered, washed with acetone and dried in vacuo to afford the title compound (85.0 mg, 0.16 mmol, 13%) as an beige solid.

m/z 401.2/403.5 (M+H)⁺ (ES+)

¹H NMR (500 MHz, DMSO-d₆) δ 11.13 (s, 1H), 7.43-7.36 (m, 2H), 7.26 (dd, J=8.2, 0.8 Hz, 1H), 7.18 (d, J=2.3 Hz, 1H), 7.08-7.00 (m, 3H), 6.64 (dd, J=7.7, 0.8 Hz, 1H), 6.56 (s, 2H), 2.86-2.79 (m, 2H), 2.70-2.64 (m, 2H), 2.29 (s, 6H), 1.76 (s, 6H). (2× exchangeable H not observed).

### Example 28. Synthesis of compound 79: 2-(dimethylamino)-2-oxoethyl (3-(2-(dimethylamino)ethyl)-1H-indol-4-yl) carbonate, Formic Acid

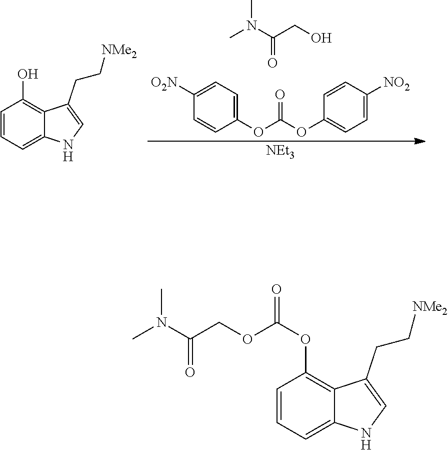

To a stirred solution of 2-hydroxy-N,N-dimethylacetamide (103 mg, 1.04 Eq, 997 μmol) and bis(4-nitrophenyl) carbonate (357 mg, 1.22 Eq, 1.18 mmol) in dry DMF (3 mL) under a nitrogen atmosphere at r.t. was added triethylamine (218 mg, 300 μL, 2.23 Eq, 2.15 mmol). The reaction mixture was stirred at r.t. for 2 h. A solution of psilocin (200.8 mg, 1 Eq, 963 μmol) in DMF (1 mL) was added. The reaction mixture was heated at 40° C. o.n. The reaction mixture was cooled to r.t., diluted with DCM (10 mL) and water (10 mL) was added. The aqueous layer was extracted with DCM (3×25 mL). The combined organic layers were washed with brine, dried over Na₂SO₄, filtered and concentrated in vacuo. The crude product was purified by chromatography on RP Flash C18 (24 g cartridge, 0-30% (0.1% formic acid in MeCN)/(0.1% formic acid in water)) to afford partially purified title compound (96.6 mg, 0.22 mmol, 22%) as a brown oil. The product was dissolved in acetone (5 mL). fumaric acid (34 mg, 0.306 Eq, 295 μmol) in acetone (5 mL) was added. The mixture was left in the freezer for 1 week and no solid was formed. The volatiles were removed in vacuo and the residue was dissolved in DMSO (1.93 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mLmin⁻¹ eluting with a 0.1% formic acid in water-MeCN gradient over 12.5 min using UV across all wavelengths with PDA as well as a QDA and ELS detector. At column dilution pump gives 2 mLmin⁻¹ MeCN over the entire method, which is included in the following MCCN percentages. Gradient information: 0.0-0.5 min, 5% MeCN; 0.5-10.5 min, ramped from 5% MeCN to 22.5% MeCN; 10.5-10.6 min, ramped from 22.5% MeCN to 100% MeCN; 10.6-12.5 min, held at 100% MeCN. The clean fractions were evaporated in a Genevac to afford the title compound (61.7 mg, 0.15 mmol, 15%) as a brown oil.

m/z 334.2 (M+H)⁺ (ES+)

¹H NMR (500 MHz, DMSO-d₆) δ 11.11 (s, 1H), 8.22 (s, 1H), 7.26 (d, J=8.2 Hz, 1H), 7.19 (s, 1H), 7.06 (dd, J=7.9, 7.9 Hz, 1H), 6.76 (d, J=7.8 Hz, 1H), 4.95 (s, 2H), 2.94-2.90 (m, 5H), 2.85 (s, 3H), 2.70-2.59 (m, 2H), 2.34 (s, 6H). 1× exchangeable H not observed.

### Example 29. Synthesis of compound 82: 2-(benzyl(methyl)amino)ethyl (3-(2-(dimethylamino)ethyl)-1H-indol-4-yl) carbonate, Formic Acid

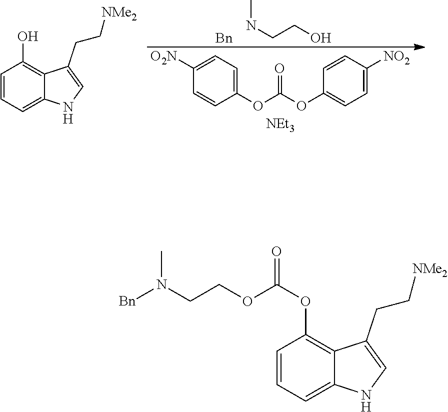

To a solution of 2-(benzyl(methyl)amino)ethan-1-ol (125 mg, 0.8 Eq, 755 μmol) and bis(4-nitrophenyl) carbonate (301 mg, 1.1 Eq, 990 μmol) in dry DMF (2 mL) under a nitrogen atmosphere at r.t. was added DIPEA (742 mg, 1.0 mL, 6.1 Eq, 5.74 mmol). The reaction mixture was stirred for 1.5 h at r.t. To the reaction mixture was added a solution of psilocin (202.8 mg, 1 Eq, 943 μmol) in DMF (2 mL), followed by DMAP (12 mg, 0.1 Eq, 98.2 μmol). The reaction mixture was stirred at r.t. and monitored over a period of 5 days. The reaction mixture was diluted with water (25 mL). The layer was extracted with DCM (3×20 mL). The combined organic layers were collected, dried (Na₂SO₄) filtered and concentrated in vacuo. The crude product was loaded onto celite and purified by chromatography on RP Flash C18 (12 g cartridge, 5-30% (0.1% formic acid in MeCN)/(0.1% formic acid in water)) to afford partially purified target compound (307 mg). The sample was dissolved in DMSO (3.32 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mLmin⁻¹ eluting with a 0.1% formic acid in water-MeCN gradient over 12.5 min using UV across all wavelengths with PDA as well as a QDA and ELS detector. At column dilution pump gives 2 mLmin⁻¹ MeCN over the entire method, which is included in the following MeCN percentages. Gradient information: 0.0-0.5 min, 5% MeCN; 0.5-10.5 min, ramped from 5% MeCN to 20% MeCN; 10.5-10.6 min, ramped from 20% MeCN to 100% MeCN: 10.6-12.5 min, held at 100% MeCN. The clean fractions were evaporated in a Genevac. The partially purified sample was dissolved in DMSO (2 mL), filtered and purified by reversed phase preparative HPLC (Gilson) using a Phenomenex Gemini NC—C18 prep column, 110 Å, 5 μm, 30 mm×150 mm, flow rate 42 mLmin⁻¹ eluting with a 0.1% formic acid in water-MeCN gradient over 15 min. At column dilution pump gives 5 mLmin⁻¹ 10% MeCN in water for 1.2 min. Gradient information: 0.0-0.5 min, 1% MeCN; 0.5-15.0 min, ramped from 1% MeCN to 14.9% MeCN; 15.0-15.1 min, ramped from 14.9% MeCN to 100% MeCN; 15.1-17.0 min, held at 100% MeCN. The clean fractions were evaporated in a Genevac to afford the title compound (16.0 mg, 31 μmol, 3%) as a sticky brown gum.

m/z 396.5 (M+H)⁺ (ES+)

¹H NMR (500 MHz, DMSO-d₆) δ 11.18-11.05 (m, 1H), 8.25 (s, 1H), 7.32-7.30 (m, 4H), 7.29-7.22 (m, 2H), 7.19 (d, J=2.3 Hz, 1H), 7.05 (dd, J=7.9 Hz, 1H), 6.76 (dd, J=7.7, 0.8 Hz, 1H), 4.34 (t, J=5.7 Hz, 2H), 3.54 (s, 2H), 2.82 (dd, J=9.2, 6.7 Hz, 2H), 2.70 (t, J=5.7 Hz, 2H), 2.60-2.55 (m, 2H), 2.26 (s, 6H), 2.19 (s, 3H). 1× exchangeable H not observed.

### Example 30. Synthesis of compound 83: 3-(2-(dimethylamino)ethyl)-1H-indol-4-yl (2,5,8,11-tetraoxatridecan-13-yl) carbonate, 0.9Formic Acid, 0.1Fumaric acid

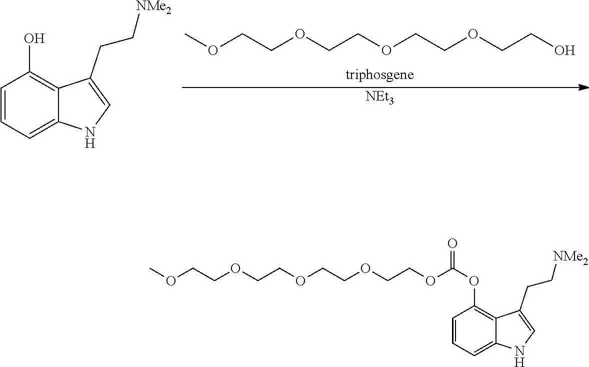 [SMILES: CN(C)CCc1c[nH]c2cccc(O)c12 ; COCCOCCOCCOCCO ; COCCOCCOCCOCCOC(=O)Oc1cccc2[nH]cc(CCN(C)C)c12 ; "NEt3"]

To a solution of 2,5,8,11-tetraoxatridecan-13-ol (310 mg, 1.5 Eq, 1.49 mmol) and triethylamine (0.7 g, 1 mL, 7 Eq, 7 mmol) in THF (3 mL) at 0° C. was added triphosgene (160 mg, 0.5 Eq, 518 μmol). The reaction was stirred for 2 h at 0° C. A suspension of psilocin (202.5 mg, 1 Eq, 991 μmol) in THF (3 mL) was added at 0° C. The mixture was stirred for 2 h at 0° C. then a further 16 h at r.t. The mixture was diluted with EtOAc (10 mL) and water (10 mL). The phases were separated and the aqueous was further extracted with EtOAc (10 mL). The combined organics were washed with brine (20 mL), dried (Na₂SO₄) and concentrated in vacuo. The crude product was purified by chromatography on RP Flash C18 (24 g cartridge, 5-10% (0.1% formic acid in MeCN)/(0.1% formic acid in water)) (eluting 10%) to afford 3-(2-(dimethylamino)ethyl)-1H-indol-4-yl (2,5,8,11-tetraoxatridecan-13-yl) carbonate, formic acid (55.0 mg, 103 μmol, 10%) as a thick brown oil. The material was dissolved in acetone (3 mL) and a solution of fumaric acid (16 mg, 1.2 Eq, 138 μmol) in acetone (3 mL). The mixture was cooled at −20° C. for 100 h. The mixture was concentrated in vacuo. The crude material was dissolved in DMSO (2.1 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mLmin⁻¹ eluting with a 0.1% formic acid in water-MeCN gradient over 8.5 min using UV across all wavelengths with PDA as well as a QDA and ELS detector. At column dilution pump gives 2 mLmin⁻¹ MeCN over the entire method, which is included in the following MeCN percentages. Gradient information: 0.0-0.5 min, 7.5% MeCN; 0.5-5.5 min, ramped from 7.5% MeCN to 37.5% MeCN; 5.5-5.6 min, ramped from 37.5% MeCN to 100% MeCN; 5.6-8.5 min, held at 100% MeCN. The clean fractions were evaporated in a Genevac to afford the title compound (33.0 mg, 66 μmol, 7%) as a brown oil.

m/z 439.2 (M+H)⁻ (ES+)

¹H NMR (500 MHz, DMSO-d₆) δ 11.10 (s, 1H), 8.18 (s, 0.9H), 7.26 (d, J=8.1 Hz, 1H), 7.18 (d, J=2.3 Hz, 1H), 7.05 (dd, J=7.9, 7.9 Hz, 1H), 6.77 (d, J=7.6 Hz, 1H), 6.50 (s, 0.2H), 4.34 (dd, J=5.7, 3.4 Hz, 2H), 3.73-3.65 (m, 2H), 3.60-3.48 (m, 10H), 3.42 (dd, J=5.9, 3.7 Hz, 2H), 3.23 (s, 3H), 2.82-2.76 (m, 2H), 2.57-2.52 (m, 2H), 2.26 (s, 6H). 2× exchangeable H not observed.

### Example 31. Synthesis of compound 87: 3-(2-(dimethylamino)ethyl)-1H-indol-4-yl (2-(N-methylacetamido)ethyl) carbonate, Formic Acid

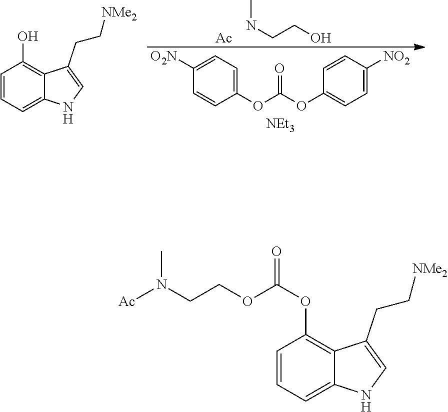

To a stirred solution of N-(2-hydroxyethyl)-N-methylacetamide (136 mg, 1.2 Eq, 1.16 mmol) and bis(4-nitrophenyl) carbonate (411 mg, 1.4 Eq, 1.35 mmol) in dry DCM (8 mL) under a nitrogen atmosphere at 20° C. was added triethylamine (234 mg, 323 μL, 2.4 Eq, 2.31 mmol). The reaction mixture was stirred for 1 h at r.t. To the reaction mixture under a nitrogen atmosphere at 20° C. was added a solution of psilocin (197 mg, 1 Eq, 964 μmol) in DMF (2 mL). The reaction mixture was stirred for 18 h at rt. The reaction mixture was then heated at 40° C. for 24 h. The reaction mixture was cooled to r.t. and was diluted with DCM (10 mL), poured into ice/water (20 mL) and transferred into a separating funnel. The aqueous layer was extracted with DCM (3×10 mL). The combined organic layers were washed with brine (15 mL), dried (Na₂SO₄), filtered and concentrated in vacuo. The material was dissolved in DMSO (5.1 mL), filtered and purified by reversed phase preparative HPLC (Waters 2767 Sample Manager, Waters 2545 Binary Gradient Module, Waters Systems Fluidics Organiser, Waters 515 ACD pump, Waters 515 Makeup pump, Waters 2998 Photodiode Array Detector, Waters QDa) on a Waters X-Select CSH C18 ODB prep column, 130 Å, 5 μm, 30 mm×100 mm, flow rate 40 mLmin⁻¹ eluting with a 0.1% formic acid in water-MeCN gradient over 17.5 min using UV across all wavelengths with PDA as well as a QDA and ELS detector. At column dilution pump gives 2 mLmin⁻¹ MeCN over the entire method, which is included in the following MeCN percentages. Gradient information: 0.0-0.5 min, 5% MeCN; 0.5-15.5 min, ramped from 5% MeCN to 17.5% MeCN; 15.5-15.6 min, ramped from 17.5% MeCN to 100% MeCN; 15.6-17.5 min, held at 100% McCN. The clean fractions were evaporated in a Genevac to afford the title compound (74.0 mg, 0.18 mmol, 19%) as a brown oil.

m/z 348.1 (M+H)⁺ (ES+)

¹H NMR (500 MHz, DMSO-d₆) δ 11.10 (s, 1H), 8.20 (s, 1H), 7.26 (dd, J=7.9, 2.5 Hz, 1H), 7.18 (d, J=2.1 Hz, 1H), 7.08-7.01 (m, 1H), 6.80-6.73 (m, 1H), 4.38 (t, J=5.3 Hz, 1H), 4.29 (t, J=5.5 Hz, 1H), 3.60 (t, J=5.5 Hz, 1H), 3.65 (t, J=5.3 Hz, 1H), 3.00 (s, 2H), 2.83 (s, 1H), 2.81-2.76 (m, 2H), 2.55-2.51 (m, 2H), 2.24 (s, 3H), 2.22 (s, 3H), 2.00 (d, J=3.3 Hz, 3H). 1× exchangeable H not observed.

### Example 32. Synthesis of compound 89: 2,4-dichlorobenzyl (3-(2-(dimethylamino)ethyl)-1H-indol-4-yl) carbonate, Formic Acid

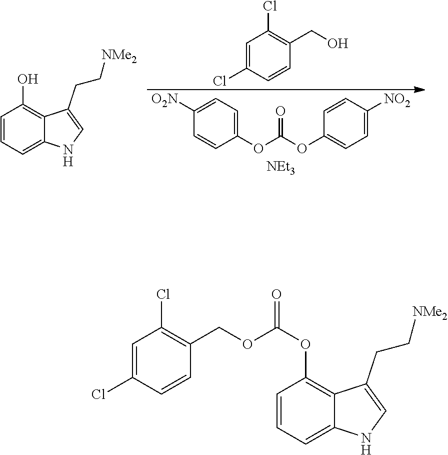

To a stirred solution of (2,4-dichlorophenyl)methanol (200 mg, 1.1 Eq, 1.13 mmol) and bis(4-nitrophenyl) carbonate (376 mg, 1.2 Eq, 1.23 mmol) in dry DCM (8 mL) under a nitrogen atmosphere at 20° C. was added triethylamine (229 mg, 316 μL, 2.2 Eq, 2.26 mmol). The reaction mixture was stirred for 1 h at r.t. To the reaction mixture under a nitrogen atmosphere at 20° C. was added a solution of psilocin (210.2 mg, 1 Eq, 1.03 mmol) in DMF (2 mL). The reaction mixture was stirred for 1 h at rt. The reaction mixture was then heated at 38° C. for 18 h. The reaction mixture was diluted with DCM (10 mL), poured into ice/water (20 mL) and transferred into a separating funnel. The aqueous layer was extracted with DCM (3×10 mL). The combined organic layers were washed with brine (15 mL), dried (Na₂SO₄), filtered and concentrated in vacuo. The crude product was purified by chromatography on silica gel (12 g cartridge, 0-20% MeOH/DCM) to afford a brown oil. The partially purified material was purified by chromatography on RP Flash C18 (12 g cartridge, 0-50% (0.1% formic acid in MeCN)/(0.1% formic acid in water)) to afford the title compound (69.1 mg, 156 μmol, 15%) as a brown oil.

m/z 407.2/409.2 (M+H)⁺ (ES+)

¹H NMR (500 MHz, DMSO-d₆) δ 11.12 (s, 1H), 8.19 (s, 1H), 7.74 (d, J=2.1 Hz, 1H), 7.64 (d, J=8.3 Hz, 1H), 7.52 (dd, J=8.2, 2.2 Hz, 1H), 7.27 (dd, J=8.1, 0.8 Hz, 1H), 7.18 (d, J=2.3 Hz, 1H), 7.09-7.02 (m, 1H), 6.80 (dd, J=7.7, 0.8 Hz, 1H), 5.36 (s, 2H), 2.78-2.71 (m, 2H), 2.56-2.51 (m, 2H), 2.19 (s, 6H). 1× exchangeable H not observed.

### Example 33. Synthesis of compound 91: 3-(2-(dimethylamino)ethyl)-1H-indol-4-yl nicotinate, Formic Acid

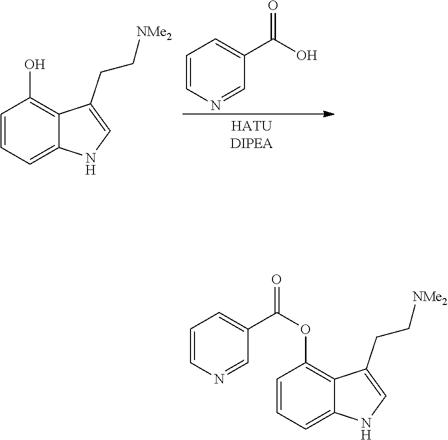 [SMILES: CN(C)CCc1c[nH]c2cccc(O)c12 ; O=C(O)c1cccnc1 ; CN(C)CCc1c[nH]c2cccc(OC(=O)c3cccnc3)c12]

A mixture of psilocin (200.0 mg, 1 Eq, 852 μmol), nicotinic acid (106 mg, 1 Eq, 852 μmol), HATU (466 mg, 1.4 Eq, 1.23 mmol) and diisopropylethylamine (260 mg, 347 μL, 2.4 Eq, 2.01 mmol) in dry DMF (2.5 mL) was stirred at r.t. for 16 h. The reaction mixture was diluted with sat. aq. NaHCO₃(25 mL) and extracted with EtOAc (3×25 mL). The combined organic layers were washed with water (3×20 mL) and brine (25 mL), dried (Na₂SO₄), filtered, and concentrated in vacuo. The crude product was loaded onto celite and purified by chromatography on RP Flash C18 (12 g cartridge, 0-100% (0.1% formic acid in MeCN)/(0.1% formic Acid in Water)) to afford the title compound (150.6 mg, 0.41 mmol, 48%) as a light brown solid.

m/c 310.1 (M+H)⁺ (ES+)

¹H NMR (500 MHz, DMSO-d₆) δ 11.16 (s, 1H), 9.35 (d, J=2.2 Hz, 1H), 8.92 (dd, J=4.8, 1.7 Hz, 1H), 8.62-8.51 (m, 1H), 8.21 (s, 1H), 7.68 (dd, J=8.0, 4.8 Hz, 1H), 7.32 (d, J=8.1 Hz, 1H), 7.20 (d, J=2.3 Hz, 1H), 7.16-7.08 (m, 1H), 6.86 (d, J=7.6 Hz, 1H), 2.79-2.72 (m, 2H), 2.60-2.53 (m, 2H), 2.06 (s, 6H). 1× exchangeable H not observed.

### Example 34. Synthesis of compound 96: 3-(2-(dimethylamino)ethyl)-1H-indol-4-yl 4-phenylbutanoate, 0.5Fumaric acid

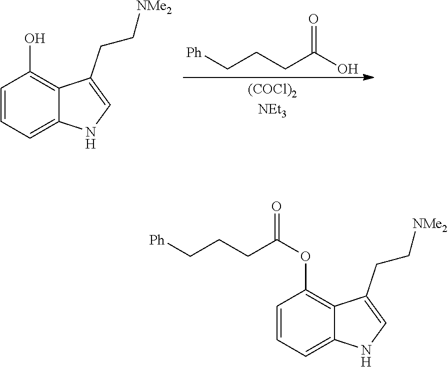 [SMILES: CN(C)CCc1c[nH]c2cccc(O)c12 ; O=C(O)CCCc1ccccc1 ; CN(C)CCc1c[nH]c2cccc(OC(=O)CCCc3ccccc3)c12]

To a stirred solution of 4-phenylbutanoic acid (141 mg, 1 Eq, 860 μmol) in dry DCM (4 mL) under an atmosphere of nitrogen at r.t. was added oxalyl chloride (229 mg, 158 μL, 2.1 Eq, 1.81 mmol) and a drop of DMF. The reaction mixture was stirred at room temp for 2 h. The volatiles were removed in vacuo. The residue was dissolved in DCM (2 mL) and added to a solution of psilocin (199.6 mg, 1 Eq, 860 μmol) and triethylamine (313 mg, 431 μL, 3.6 Eq, 3.10 mmol) in DCM (2 mL) at 0° C. The reaction mixture was stirred at r.t. for 1 h. The reaction mixture was diluted with water (5 mL) and transferred into a separating funnel. The aqueous layer was extracted with DCM (3×5 mL). The combined organic layers were collected, dried (Na₂SO₄), filtered and concentrated in vacuo. The crude product was purified by chromatography on RP Flash C18 (24 g cartridge, 5-50% (0.1% formic acid in MeCN)/(0.1% formic acid in water)) to afford 3-(2-(dimethylamino)ethyl)-1H-indol-4-yl 4-phenylbutanoate, formic acid (144 mg, 0.30 mmol, 35%) as a brown oil. The material was dissolved in acetone (2 mL) and a solution of fumaric acid (35 mg, 0.35 Eq, 0.30 mmol) in acetone (4 mL) was added. The resulting solid was filtered, washed with acetone (2 mL) and dried in a vacuum desiccator overnight to afford the title compound (56.8 mg, 137 μmol, 16%) as a light brown solid.

m/z 351.0 (M+H)⁺ (ES+)

¹H NMR (500 MHz, DMSO) δ 11.08 (d, J=2.5 Hz, 1H), 7.34-7.27 (m, 2H), 7.26-7.18 (m, 4H), 7.16 (d, J=2.3 Hz, 1H), 7.04 (dd, J=7.9 Hz, 1H), 6.66 (dd, J=7.6, 0.8 Hz, 1H), 6.53 (s, 1H), 2.81 (dd, J=9.4, 6.6 Hz, 2H), 2.73-2.67 (m, 4H), 2.64 (dd, J=9.6, 6.3 Hz, 2H), 2.31 (s, 6H), 1.99 (app. p, J=7.6 Hz, 2H). (2×0.5 exchangeable H not observed)

### Example 35. Synthesis of compound 97: 3-(2-(Dimethylamino)ethyl)-1H-indol-4-yl 4-acetamidobenzoate, 0.5 Fumaric acid

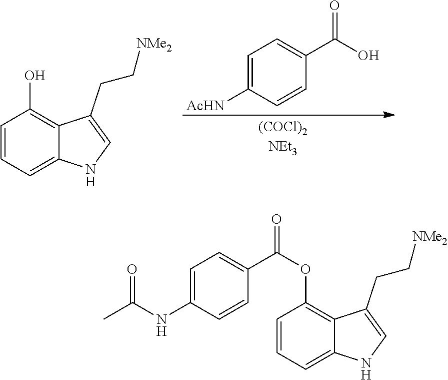 [SMILES: CN(C)CCc1c[nH]c2cccc(O)c12 ; CC(=O)Nc1ccc(C(=O)O)cc1 ; CC(=O)Nc1ccc(C(=O)Oc2cccc3[nH]cc(CCN(C)C)c23)cc1]

To a stirred solution of 4-acetamidobenzoic acid (182.6 mg, 1 Eq, 1.019 mmol) in dry DCM (4.00 mL) under an atmosphere of N₂ at rt was added oxalyl chloride (271.7 mg, 187.4 μL, 2.1 Eq, 2.140 mmol) and a drop of DMF (74.51 mg, 78.9 μL, 1 Eq, 1.019 mmol). The reaction mixture was stirred at rt for 2 h. The volatiles were removed in vacuo. The residue was dissolved in DCM (2.00 mL) and added to a solution of psilocin (208.2 mg, 1 Eq, 1.019 mmol) and Et₃N (371.3 mg, 511 μL, 3.6 Eq, 3.669 mmol) in DCM (2.00 mL) at 0° C. The reaction mixture was stirred at rt for 1 h. The reaction mixture was concentrated in vacuo. The crude product (diluted in 1.5 mL of DMF) was purified by chromatography on RP Flash C18 (12 g cartridge, 5-50% (0.1% formic acid in MeCN)/(0.1% formic acid in water)) to afford impure material (220 mg, 0.40 mmol, 39%, 75% Purity) as a brown oil. The material was subjected to a second purification on RP Flash C18 (12 g cartridge, 5-50% (0.1% formic acid in MeCN)/(0.1% formic acid in water)) to afford the desired material as the formate salt (76.0 mg, 0.15 mmol, 14%, 80% Purity (20% acetonitrile)) as a brown oil. The material was dissolved in acetone (5 mL) and a solution of fumaric acid (17 mg, 0.15 Eq, 0.15 mmol) in acetone (5 mL) was added. The resulting solid was filtered, washed with acetone and dried in a vacuum desiccator over the weekend to afford the title compound (44.7 mg, 97.6 smol, 9.58%, 92.5% Purity) as a yellow solid.

m/z 366.19 (M+H)⁺ (ES+); 364.19 (M−H)⁻ (ES−)

¹H NMR (500 MHz, DMSO) δ 11.11 (s, 1H), 10.38 (s, 1H), 8.16-8.12 (m, 2H), 7.84-7.78 (m, 2H), 7.28 (dd, J=8.1, 0.8 Hz, 1H), 7.17 (d, J=2.3 Hz, 1H), 7.12-7.05 (m, 1H), 6.77 (dd, J=7.6, 0.8 Hz, 1H), 6.53 (s, 1H), 2.77-2.70 (m, 2H), 2.58-2.51 (m, 2H), 2.11 (s, 3H), 2.06 (s, 6H). 1H not observed (exchangeable proton of fumaric acid)

### Example 36. Synthesis of compound 99: 3-(2-(dimethylamino)ethyl)-1H-indol4-yl 2-acetoxybenzoate, Fumaric acid

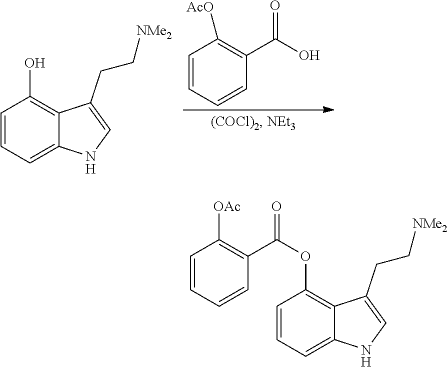 [SMILES: CN(C)CCc1c[nH]c2cccc(O)c12 ; CC(=O)Oc1ccccc1C(=O)O ; CC(=O)Oc1ccccc1C(=O)Oc1cccc2[nH]cc(CCN(C)C)c12]

To a stirred solution of 2-acetoxybenzoic acid (210 mg, 1.2 Eq, 1.17 mmol) in dry DCM (2 mL) under a nitrogen atmosphere at r.t. was added oxalyl chloride (149 mg, 103 μL, 1.2 Eq, 1.17 mmol) and a drop of DMF. The reaction mixture was stirred at r.t. for 2 h. The volatiles were removed in vacuo. The residue was redissolved in DCM (2 mL) and added to a solution of psilocin (202.9 mg, 1 Eq, 973 μmol) and triethylamine (218 mg, 300 μL, 2.2 Eq, 2.15 mmol) in DCM (2 mL) at 0° C. The resulting mixture was stirred at r.t. for 18 h. The mixture was diluted with DCM (10 mL) poured into ice/water (20 mL). The phases were separated and the aqueous extracted with further DCM (10 mL). The combined organics were washed with brine (20 mL), dried (Na₂SO₄) and concentrated in vacuo. The crude product was purified by chromatography on RP Flash C18 (12 g cartridge, 5-50% (0.1% formic acid in MeCN)/(0.1% formic acid in water)) (eluting ˜20%) to afford 3-(2-(dimethylamino)ethyl)-1H-indol-4-yl 2-acetoxybenzoate, Formic Acid (244 mg, 592 μmol, 61%) as a sticky orange oil. To a solution of the resultant oil in acetone (2 mL) was added a solution of fumaric acid (240 mg, 2.12 Eq, 2.1 mmol) in acetone (12 mL). The mixture was stored at −20° C. for 16 h. The resulting crystalline solid was isolated by filtration and washed with cold acetone (2×5 mL) to afford the title compound (164.9 mg, 0.32 mmol, 33%) as a white crystalline solid.

m/z 367.4 (M+H)⁺ (ES+)

¹H NMR (500 MHz, DMSO-d₆) δ 12.84 (s, 2H), 11.14 (s, 1H), 8.29 (dd, J=7.8, 1.7 Hz, 1H), 7.80 (ddd, J=7.8, 7.8, 1.7 Hz, 1H), 7.52 (dd, J=7.6, 7.6 Hz, 1H), 7.32 (dd, J=24.2, 8.1 Hz, 2H), 7.19 (d, J=2.4 Hz, 1H), 7.10 (dd, J=7.9, 7.9 Hz, 1H), 6.74 (d, J=7.6 Hz, 1H), 6.56 (s, 2H), 2.78-2.69 (m, 2H), 2.58 (t, J=8.0 Hz, 2H), 2.21 (s, 3H), 2.09 (s, 6H).

### Example 37. Human Hepatocyte Clearance and Human Plasma Stability

### Human Plasma Stability Summary

The plasma stability of compounds was assessed by monitoring the disappearance of parent compounds for up to 2 hours at 37° C. in plasma in duplicate, using positive (Propantheline), negative (Pepstatin) and solvent controls (DMSO) to confirm suitability of the assay. Psilocin was also run as a control to monitor psilocin formation in a semi-quantitative manner.

Samples were analysed by UHPLC-MS/MS—Sciex™ MS500 Triple Quad QTRAP UHPLC system with a HESI-II electrospray source on a Waters™ Acquity UPLC® HSS T3 column (1.8 μm, 2.1 mm×50 mm), mobile were phases water+0.1% formic acid and methanol+0.1% formic acid.

The elimination rate constant and half-life (t1/2) were determined using Ln(MS response) vs time plots. In addition, the appearance of psilocin from test compounds was monitored and assessed (as a percentage) against control psilocin peaks (at Time 0) to provide a semi-quantitative measure of psilocin release. The human plasma stability data are included in table A.

### Human Hepatocyte Clearance

The metabolic stability of compounds was assessed by monitoring the disappearance of parent compounds for up to 1 hour at 37° C. in cryopreserved hepatocytes, using established controls (Diltazem and Naloxone), to confirm suitability of the assay. Psilocin was also run as an additional control to monitor psilocin formation in a semi-quantitative manner.

Samples were analysed by UHPLC-MS/MS—Waters™ Acquity UPLC system, Waters™ Xevo TQ-XS on a Waters™ Acquity UPLC® HSS T3 column (1.8 μm, 2.1 mm×30 mm), mobile phases were water+0.1% formic acid and methanol+0.1% formic acid.

The elimination rate constant, half-life (t1/2) and intrinsic clearance (CLᵢₙₜ, μL/min/10⁶ cells) were determined using Ln(MS response) vs time plots. In addition, the appearance of psilocin from test compounds was monitored and assessed (as a percentage) against control psilocin peaks (at Time 0) to provide a semi-quantitative measure of psilocin release. The human hepatocyte clearance plasma data are included in table A.

TABLE A
Human plasma stability data and human hepatocyte
clearance plasma data of selected compounds

| | Human Plasma | Human Hepatocytes |
|---|---|---|
| Compound_Number | Maximum % Psilocin | Maximum % Psilocin |
| 52 | 100 | 54 |
| 189 | 100 | 100 |
| 191 | 100 | 82 |
| 168 | 100 | 100 |
| 169 | 8 | 21 |
| 171 | 26 | 67 |
| 188 | 18 | 34 |
| 82 | 85 | |
| 87 | 91 | 100 |
| 89 | 100 | 100 |
| 91 | 100 | 200 |
| 99 | 100 | 100 |
| 105 | 82 | 65 |
| 107 | 81 | |
| 110 | 100 | |
| 135 | 2 | 1 |
| 139 | 100 | |
| 195 | 2 | 25 |
| 197 | | 57 |
| 123 | 100 | |
| 78 | 100 | 100 |
| 97 | 100 | |
| 113 | 100 | 100 |
| 117 | 100 | 200 |
| 126 | 12 | |
| 137 | 11 | |
| 79 | 100 | |
| 185 | 100 | |
| 100 | 100 | |
| 118 | 100 | |
| 109 | 100 | |
| 129 | 31 | |
| 176 | 100 | |
| 96 | 75 | |
| 83 | 100 | 59 |

1. A compound selected from any one of the following structures, or a pharmaceutically acceptable salt or a deuterated form thereof:

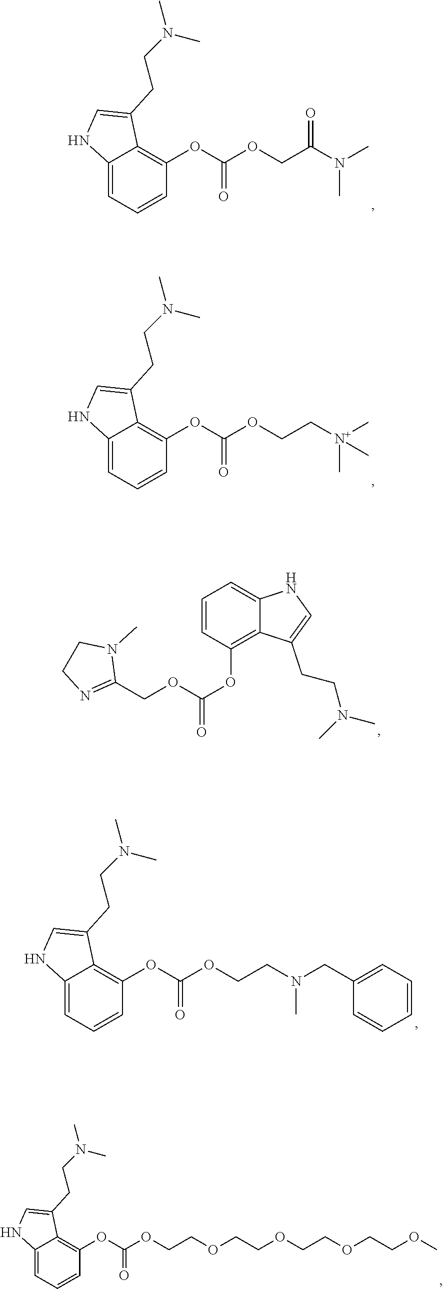 [SMILES: CN(C)CCc1c[nH]c2cccc(OC(=O)OCC(=O)N(C)C)c12 ; CN(C)CCc1c[nH]c2cccc(OC(=O)OCC[N+](C)(C)C)c12 ; CN(C)CCc1c[nH]c2cccc(OC(=O)OCC3=NCCN3C)c12 ; CN(C)CCc1c[nH]c2cccc(OC(=O)OCCN(C)Cc3ccccc3)c12 ; COCCOCCOCCOCCOC(=O)Oc1cccc2[nH]cc(CCN(C)C)c12]

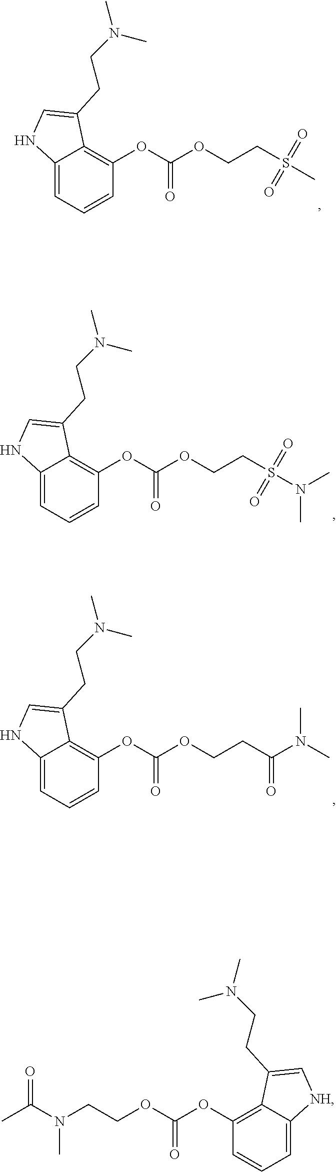

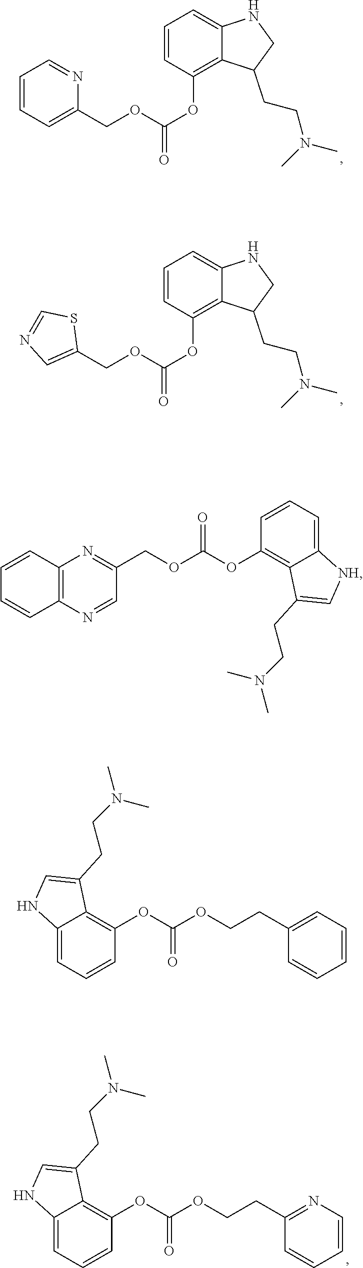 [SMILES: CN(C)CCC1CNc2cccc(OC(=O)OCc3ccccn3)c21 ; CN(C)CCC1CNc2cccc(OC(=O)OCc3cncs3)c21 ; CN(C)CCc1c[nH]c2cccc(OC(=O)OCc3cnc4ccccc4n3)c12 ; CN(C)CCc1c[nH]c2cccc(OC(=O)OCCc3ccccc3)c12 ; CN(C)CCc1c[nH]c2cccc(OC(=O)OCCc3ccccn3)c12]

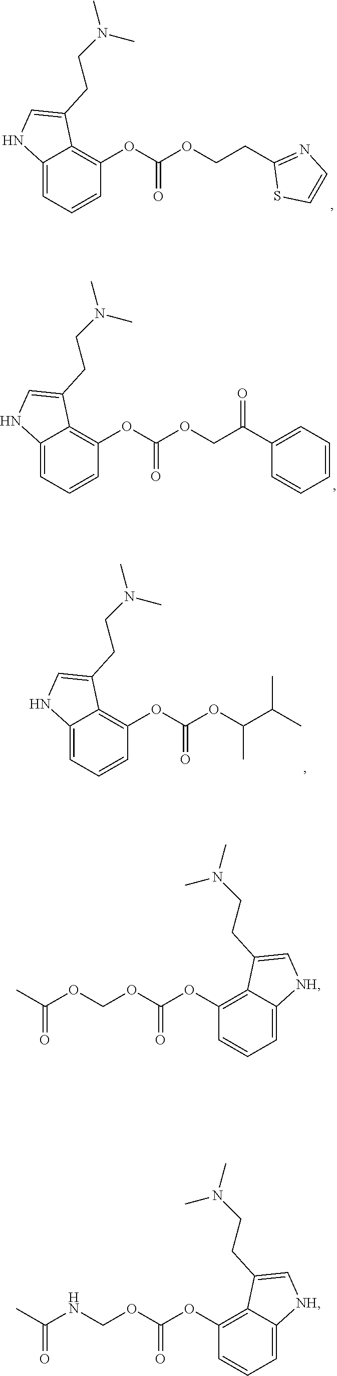

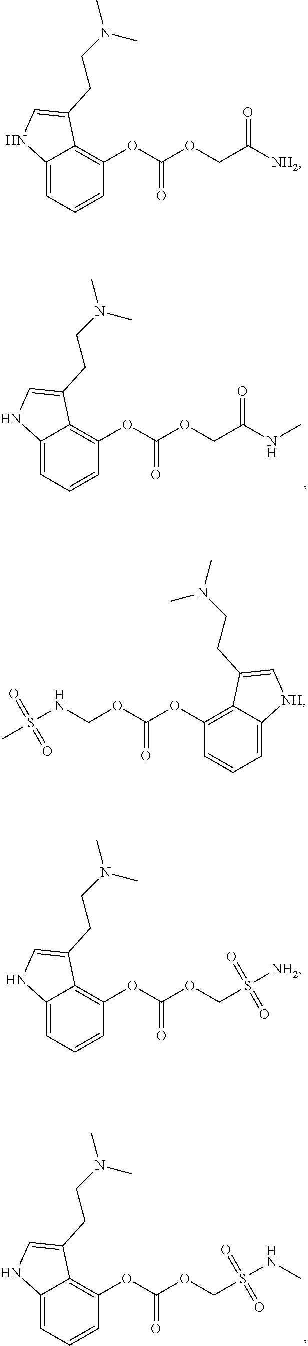

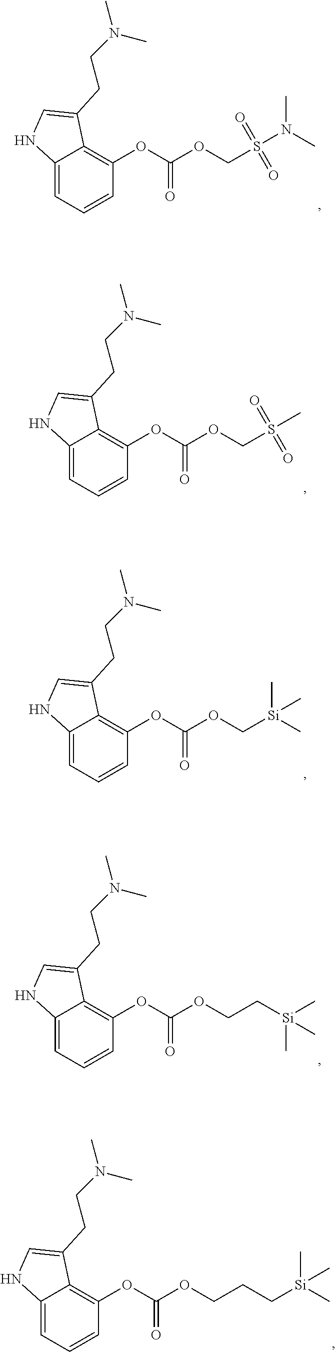 [SMILES: CN(C)CCc1c[nH]c2cccc(OC(=O)OCS(=O)(=O)N(C)C)c12 ; CN(C)CCc1c[nH]c2cccc(OC(=O)OCS(C)(=O)=O)c12 ; CN(C)CCc1c[nH]c2cccc(OC(=O)OC[Si](C)(C)C)c12 ; CN(C)CCc1c[nH]c2cccc(OC(=O)OCC[Si](C)(C)C)c12 ; CN(C)CCc1c[nH]c2cccc(OC(=O)OCCC[Si](C)(C)C)c12]

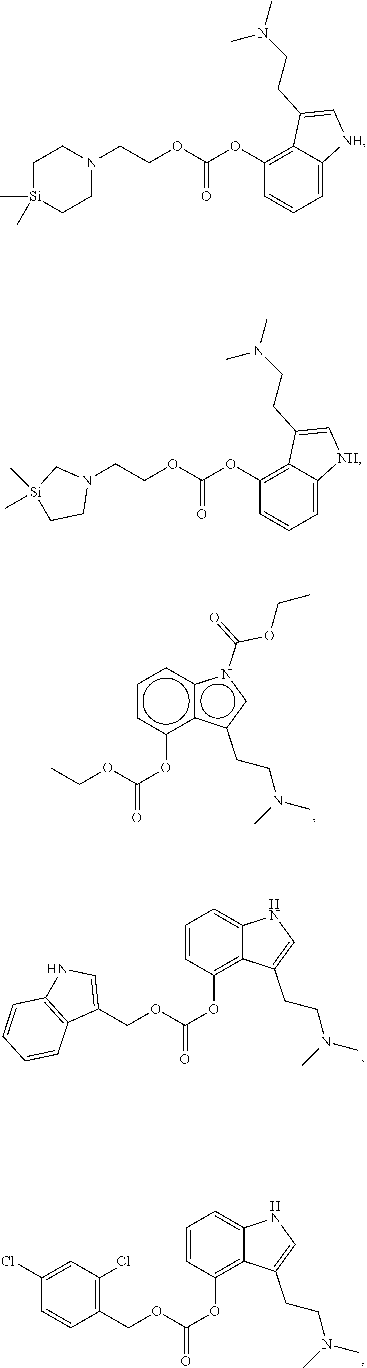

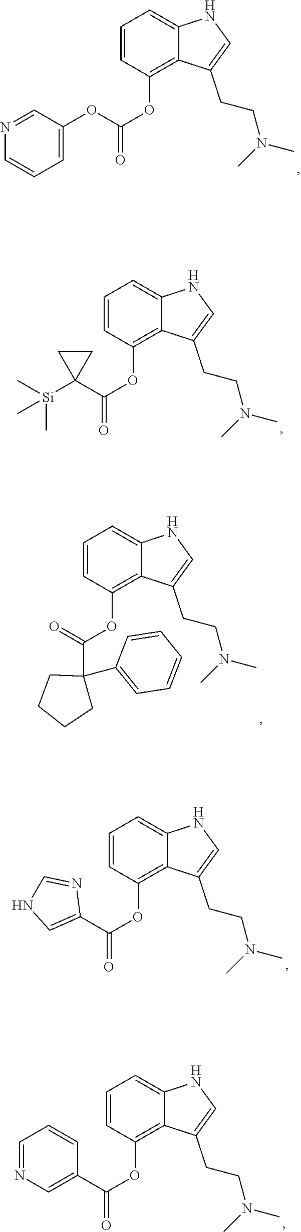

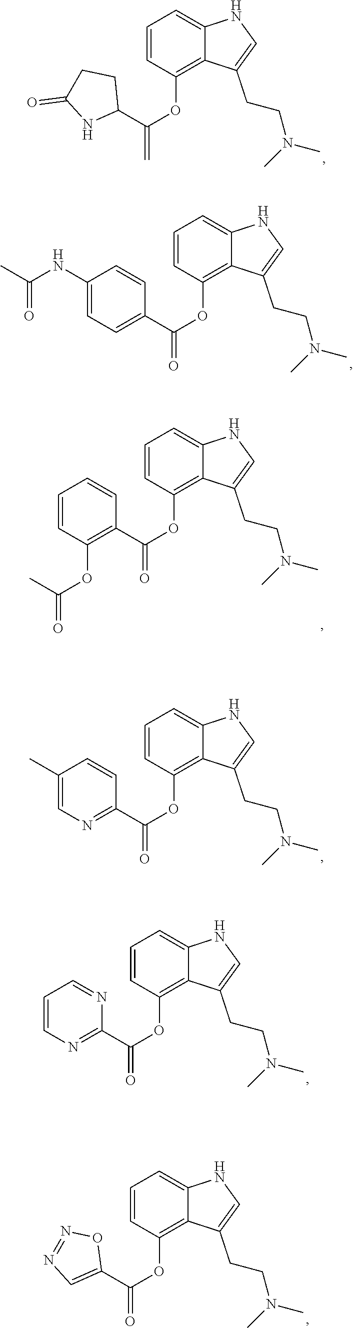

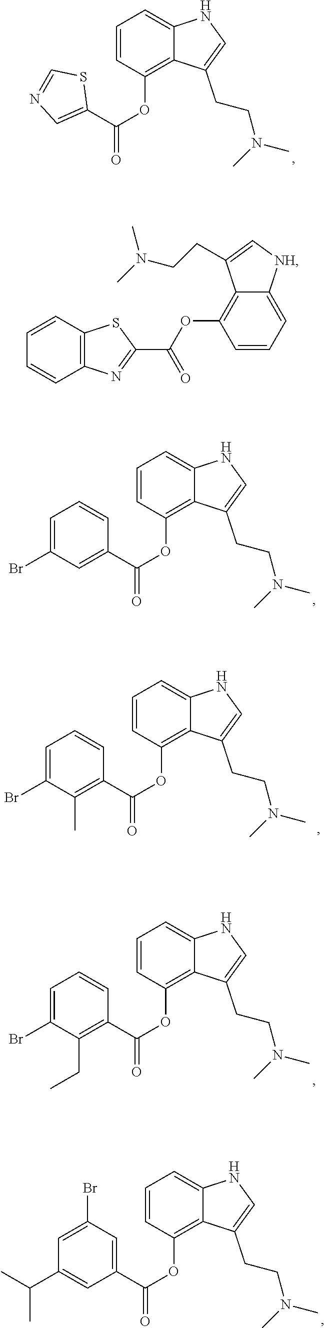

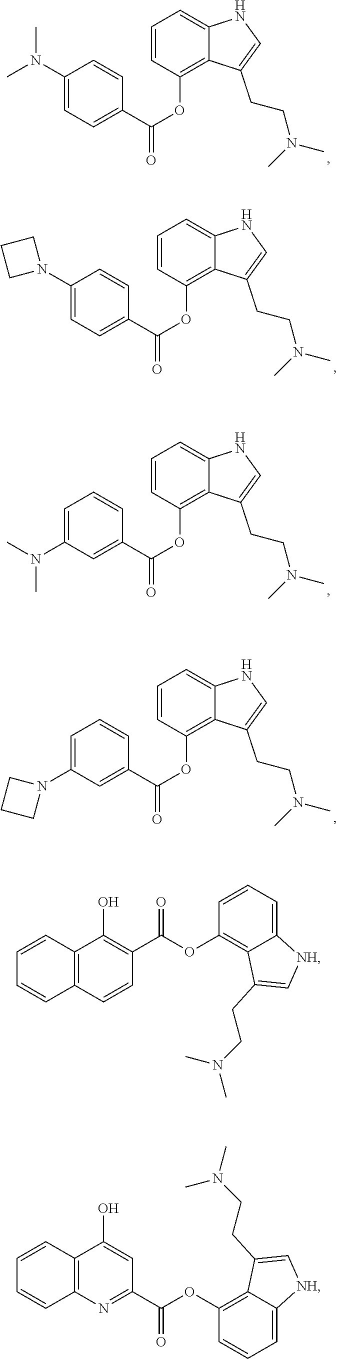

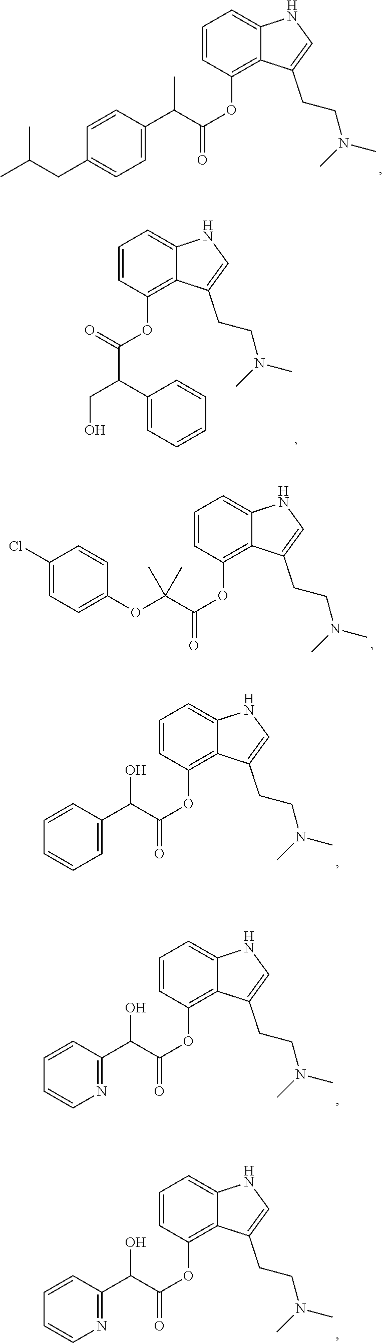 [SMILES: CC(C)Cc1ccc(C(C)C(=O)Oc2cccc3[nH]cc(CCN(C)C)c23)cc1 ; CN(C)CCc1c[nH]c2cccc(OC(=O)C(CO)c3ccccc3)c12 ; CN(C)CCc1c[nH]c2cccc(OC(=O)C(C)(C)Oc3ccc(Cl)cc3)c12 ; CN(C)CCc1c[nH]c2cccc(OC(=O)C(O)c3ccccc3)c12 ; CN(C)CCc1c[nH]c2cccc(OC(=O)C(O)c3ccccn3)c12 ; CN(C)CCc1c[nH]c2cccc(OC(=O)C(O)c3ccccn3)c12]

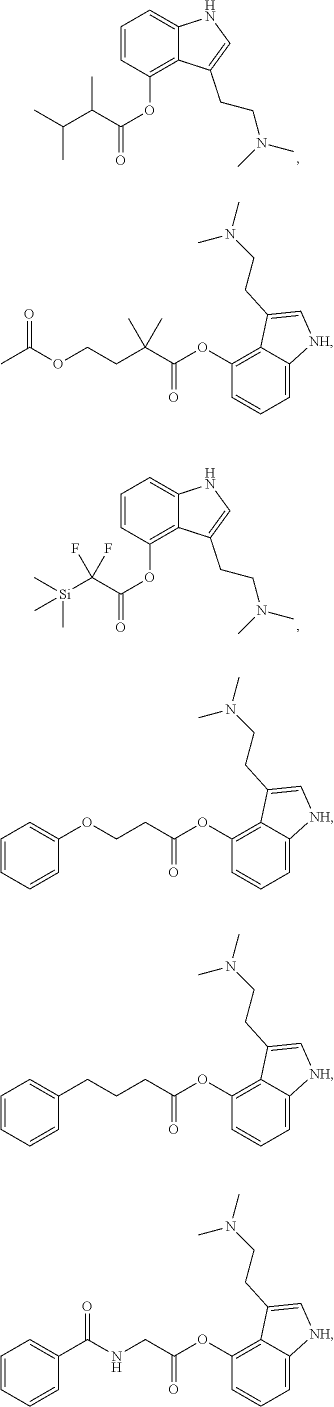

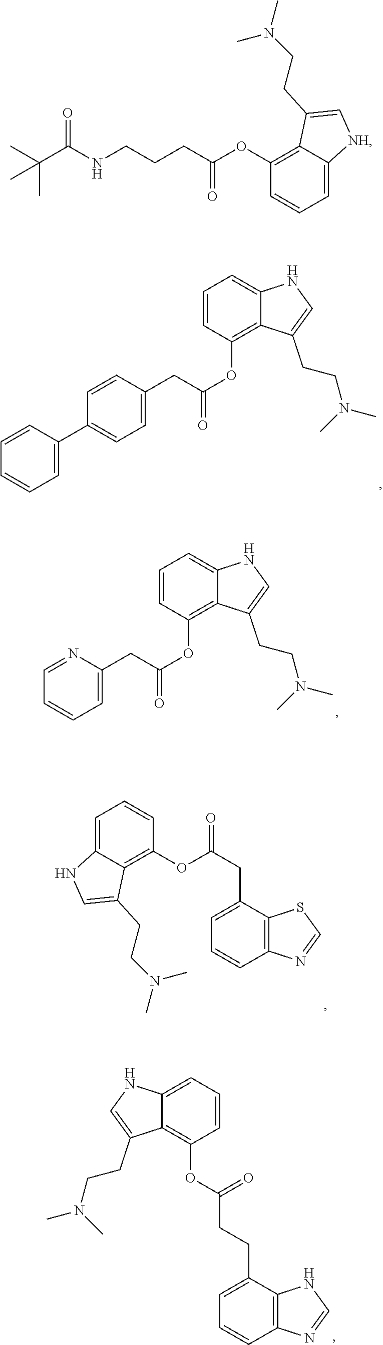

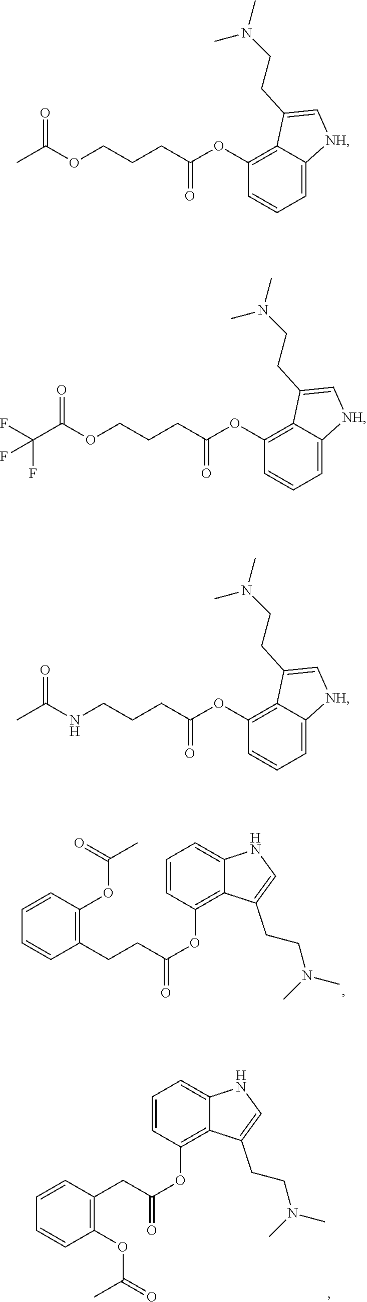 [SMILES: CC(=O)OCCCC(=O)Oc1cccc2[nH]cc(CCN(C)C)c12 ; CN(C)CCc1c[nH]c2cccc(OC(=O)CCCOC(=O)C(F)(F)F)c12 ; CC(=O)NCCCC(=O)Oc1cccc2[nH]cc(CCN(C)C)c12 ; CC(=O)Oc1ccccc1CCC(=O)Oc1cccc2[nH]cc(CCN(C)C)c12 ; CC(=O)Oc1ccccc1CC(=O)Oc1cccc2[nH]cc(CCN(C)C)c12]

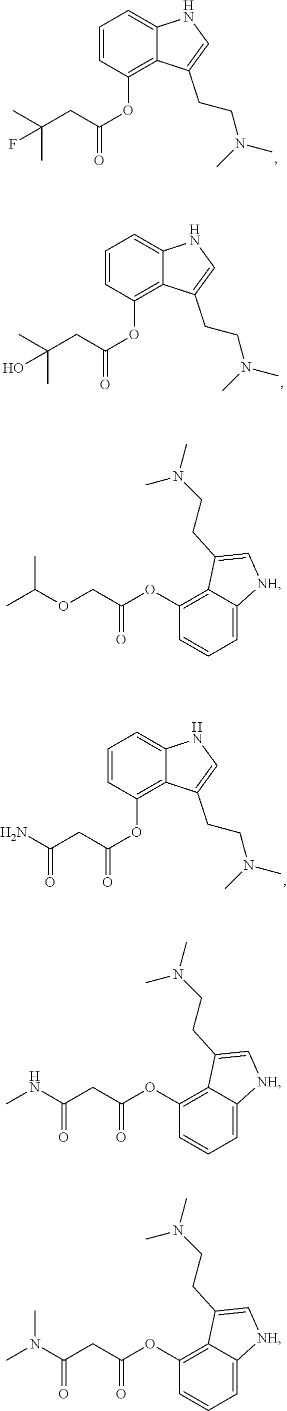

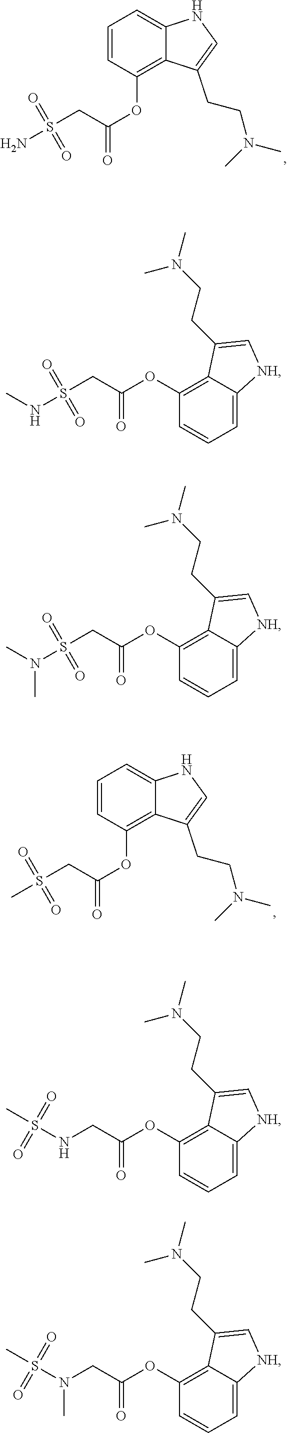

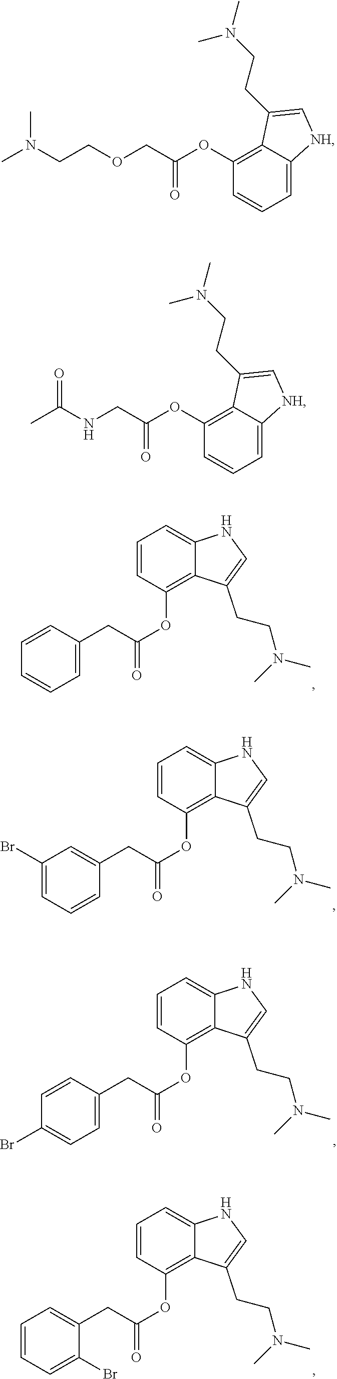 [SMILES: CN(C)CCOCC(=O)Oc1cccc2[nH]cc(CCN(C)C)c12 ; CC(=O)NCC(=O)Oc1cccc2[nH]cc(CCN(C)C)c12 ; CN(C)CCc1c[nH]c2cccc(OC(=O)Cc3ccccc3)c12 ; CN(C)CCc1c[nH]c2cccc(OC(=O)Cc3cccc(Br)c3)c12 ; CN(C)CCc1c[nH]c2cccc(OC(=O)Cc3ccc(Br)cc3)c12 ; CN(C)CCc1c[nH]c2cccc(OC(=O)Cc3ccccc3Br)c12]

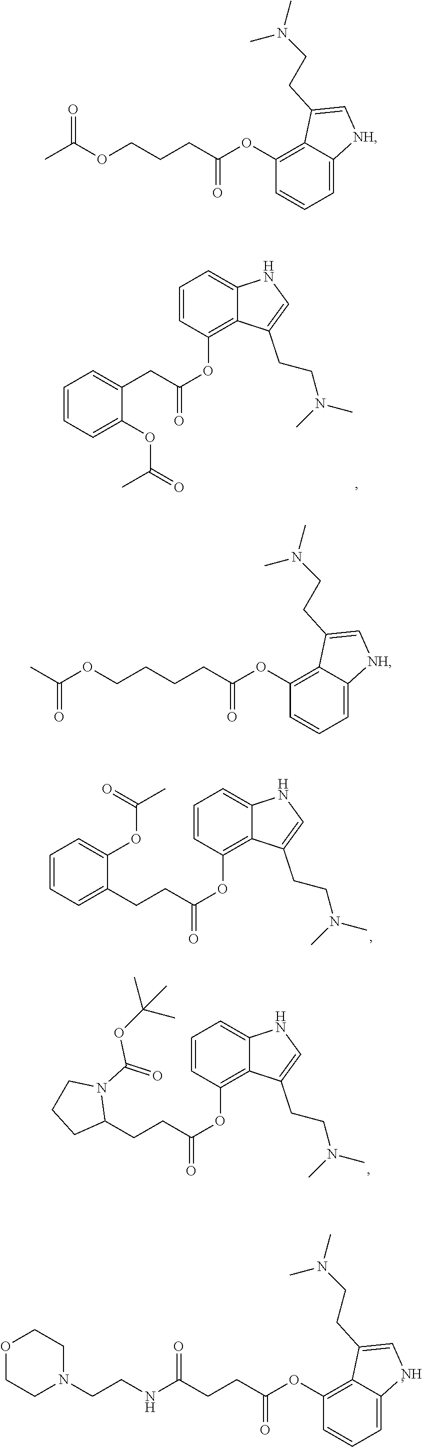

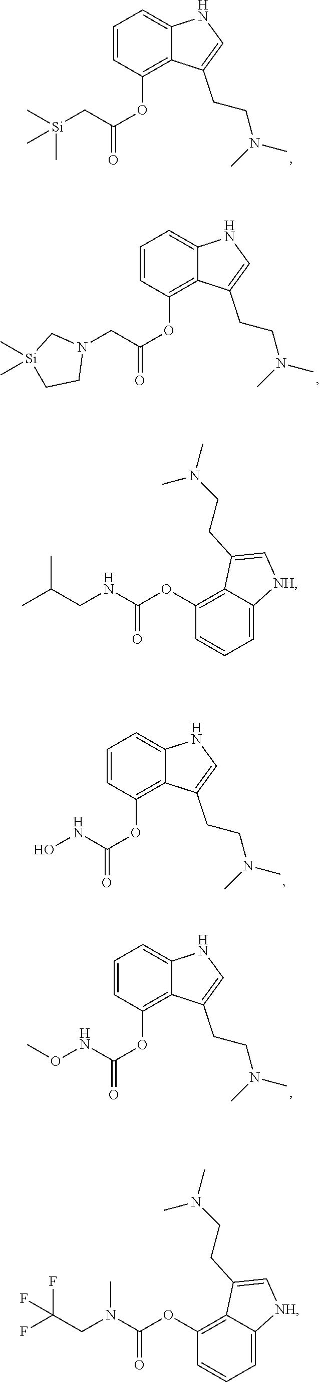

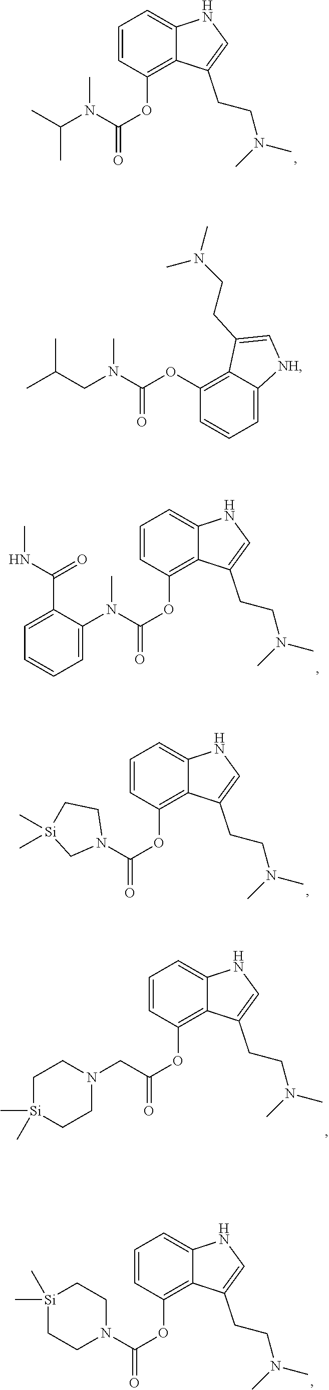

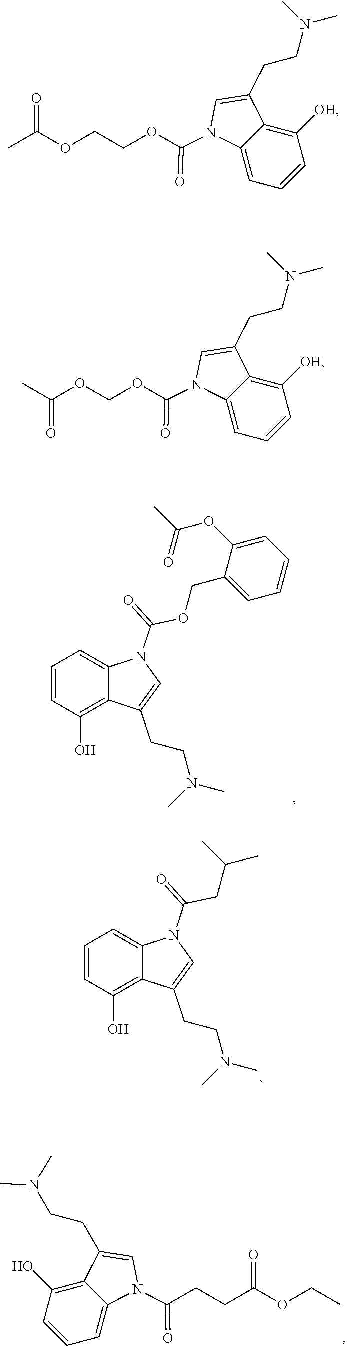

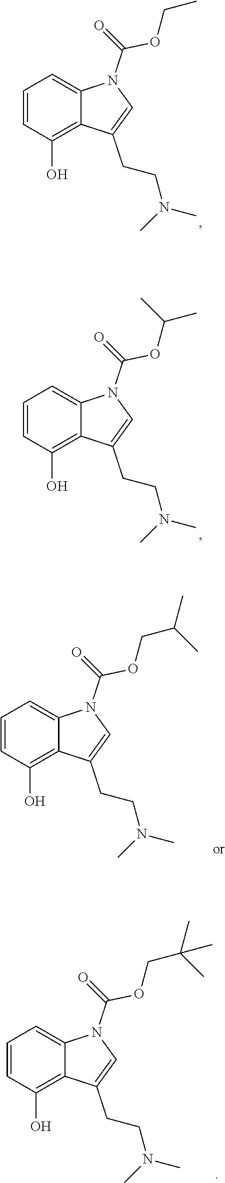 [SMILES: CCOC(=O)n1cc(CCN(C)C)c2c(O)cccc21 ; CC(C)OC(=O)n1cc(CCN(C)C)c2c(O)cccc21 ; CC(C)COC(=O)n1cc(CCN(C)C)c2c(O)cccc21 ; CN(C)CCc1cn(C(=O)OCC(C)(C)C)c2cccc(O)c12]

2. The compound of claim 1, having the following structure: 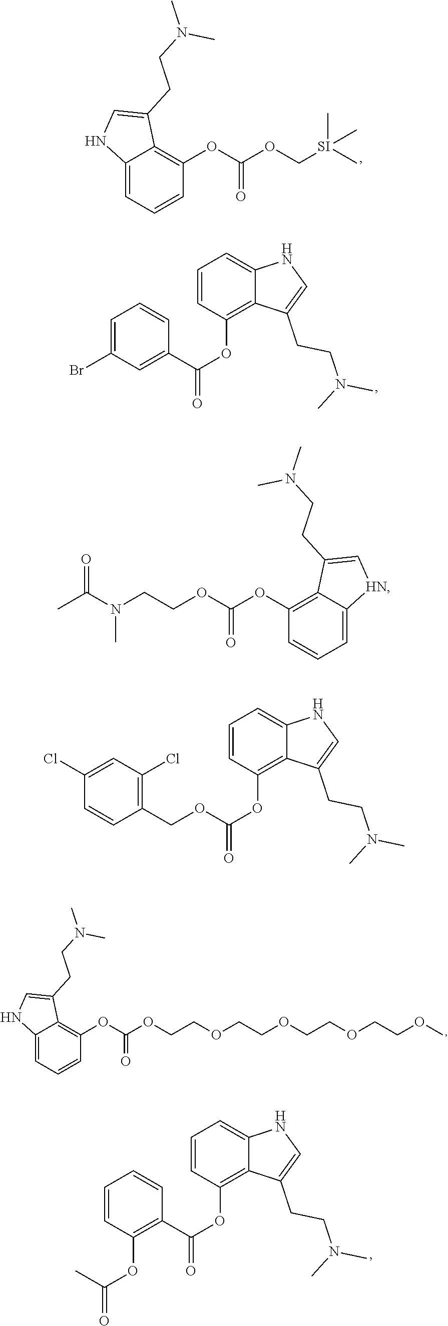 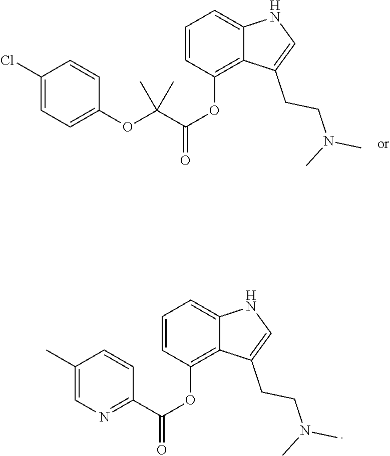
or a pharmaceutically acceptable salt or deuterated form thereof.

3. A pharmaceutical composition comprising a compound of claim 1, or a pharmaceutically acceptable salt or deuterated form thereof.

4. A method of treating 5-HT₂A receptor associated disease or disorder, comprising administering a compound of claim 1, or a pharmaceutically acceptable salt or deuterated form thereof.

5. The method of claim 4, wherein the 5-HT₂A receptor associated disease or disorder is an anxiety disorder, attention deficit hyperactivity disorder (ADHD), depression, cluster headache, diminished drive, burn-out, bore-out, migraine, Parkinson's disease, schizophrenia, an eating disorder, psychotic disorder, schizophrenia, schizophreniform disorder, schizoaffective disorder, bipolar I disorder, bipolar II disorder, major depressive disorder, psychotic depression, delusional disorders, shared psychotic disorder, Shared paranoia disorder, brief psychotic disorder, paranoid personality disorder, schizoid personality disorder, schizotypal personality disorder, social anxiety disorder, substance-induced anxiety disorder, selective mutism, panic disorder, panic attacks, agoraphobia, posttraumatic stress disorder (PTSD), premenstrual dysphoric disorder (PMDD), and premenstrual syndrome (PMS).

6. The method of claim 5, wherein the 5-HT₂A receptor associated disease or disorder is depression

7. The method of claim 6, wherein the depression is treatment resistant depression.

8. The method of claim 5, wherein the 5-HT₂A receptor associated disease or disorder is an eating disorder.

9. The method of claim 8, wherein the eating disorder is anorexia nervosa.

10. The method of claim 5, wherein the 5-HT₂A receptor associated disease or disorder is an anxiety disorder.

11. The method of claim 5, wherein the 5-HT₂A receptor associated disease or disorder is bipolar I disorder.

12. The method of claim 5, wherein the 5-HT₂A receptor associated disease or disorder is bipolar II disorder.

13. The method of claim 5, wherein the 5-HT₂A receptor associated disease or disorder is major depressive disorder.

14. The method of claim 5, wherein the 5-HT₂A receptor associated disease or disorder is posttraumatic stress disorder (PTSD).
